(19) United States
(12) Patent Application Publication
(10) Pub. No.: US 2023/0340538 A1
(43) Pub. Date: Oct. 26, 2023

(54) COMPOSITIONS AND METHODS FOR IMPROVED SITE-SPECIFIC MODIFICATION

(71) Applicant: ASTRAZENECA AB, SODERTALJE (SE)

(72) Inventor: MARCELLO MARESCA, SODERTALJE (SE)

(21) Appl. No.: 17/917,333

(22) PCT Filed: Apr. 7, 2021
(86) PCT No.: PCT/EP2021/059062

§ 371 Date: Oct. 6, 2022

Related U.S. Application Data
(60) Provisional application No. 63/006,997, filed on Apr. 8, 2020.
(60) Provisional application No. 63/104,123, filed on Oct. 22, 2020.

(51) Int. Cl. C12N 15/90; C12N 15/11; C12N 9/22; C12N 9/12; C12N 9/00; C12N 15/85

(52) U.S. Cl. CPC C12N 15/90; C12N 15/11; C12N 9/22; C12N 9/1252; C12N 9/1276; C12N 9/93; C12N 15/85; C12N 2800/107; C12N 2310/20; C12N 2800/80

(57) ABSTRACT

The present disclosure provides proteins, compositions, methods, and kits for improved gene editing efficiency. In some embodiments, the disclosure provides a fusion protein comprising a Cas nuclease and a reverse transcriptase, a DNA polymerase, a DNA ligase, or a combination thereof.

### FIELD OF THE INVENTION

The present disclosure provides proteins, compositions, methods, and kits for improved gene editing efficiency. In some embodiments, the disclosure provides a fusion protein comprising a Cas nuclease and a reverse transcriptase, a DNA polymerase, a DNA ligase, or a combination thereof.

### BACKGROUND

Programmable nucleases such as CRISPR/Cas9 can generate site-specific double-stranded breaks (DSBs) that can disrupt genes by inducing mixtures of insertions and deletions (indels) at target sites. However, DSB repair relying on the template-dependent homology-directed repair (HDR) can have low frequency, while the high efficiency template-independent non-homologous end joining (NHEJ) can be error-prone and may not favor desired insertions.

Anzalone et al. (Nature 576: 149-157 (2019)) described the development of prime editing, which utilizes a programmable nickase, which generates a single-stranded break, fused to a reverse transcriptase, which can insert short sequences at the site of cleavage. However, prime editing can only insert short sequences of up to 22 base pairs and relies upon a complex mechanism of RNA removal and hybridization of single-stranded DNA to a target site, and also requires removal of an overlapping “flap” sequence by cellular equilibrium.

### SUMMARY OF THE INVENTION

In some embodiments, the present disclosure provides a fusion protein comprising: (i) a Cas nuclease and (ii) a reverse transcriptase, a DNA polymerase, a DNA ligase, or a combination thereof, wherein the Cas nuclease is capable of generating a double-stranded polynucleotide cleavage.

In some embodiments, the disclosure provides a fusion protein comprising: (i) a Cas nuclease and (ii) a reverse transcriptase, a DNA polymerase, a DNA ligase, or a combination thereof, wherein the Cas nuclease is capable of generating a double-stranded polynucleotide cleavage.

In some embodiments, the Cas nuclease is Cas9 or Cas12. In some embodiments, the Cas9 is a Type IIB Cas9. In some embodiments, the Cas9 comprises a polypeptide sequence having at least 90% identity to SEQ ID NO: 1.

In some embodiments, the fusion protein comprises a Cas nuclease and a reverse transcriptase. In some embodiments, the reverse transcriptase is MMLV reverse transcriptase or R2 reverse transcriptase. In some embodiments, the reverse transcriptase comprises a polypeptide sequence having at least 90% identity to any one of SEQ ID NOS: 2-3.

In some embodiments, the fusion protein comprises a Cas nuclease and a DNA polymerase. In some embodiments, the DNA polymerase is phi29 DNA polymerase, T4 DNA polymerase, DNA polymerase mu, DNA polymerase delta, or DNA polymerase epsilon, Rev3, DNA polymerase I, Klenow Fragment of DNA polymerase I. In some embodiments, the DNA polymerase comprises a polypeptide sequence having at least 90% identity to any one of SEQ ID NOS: 4-6.

In some embodiments, the fusion protein comprises a Cas nuclease and a DNA ligase. In some embodiments, the DNA ligase is T4 DNA ligase. In some embodiments, the DNA ligase comprises a polypeptide sequence having at least 90% identity to SEQ ID NO: 7.

In some embodiments, the fusion protein further comprises a DNA-binding or an RNA-binding domain. In some embodiments, the DNA-binding domain is a zinc finger DNA-binding domain, a transcription factor, or an adeno-associated virus Rep protein. In some embodiments, the RNA-binding domain is MS2 coat protein (MCP2). In some embodiments, the RNA-binding domain comprises a KH domain. In some embodiments, the RNA-binding domain is heterogeneous nuclear ribonucleoprotein K (hnRNPK). In some embodiments, the DNA-binding domain is capable of binding single-stranded DNA (ssDNA). In some embodiments, the DNA-binding domain is Far upstream element-binding protein (FUBP). In some embodiments, the DNA-binding or the RNA-binding domain comprises a polypeptide sequence having at least 90% identity to any one of SEQ ID NOS: 8-11.

In some embodiments, the fusion protein further comprises a polypeptide linker between (i) and (ii).

In some embodiments, the fusion protein comprises a polypeptide sequence having at least 90% identity to any one of SEQ ID NOS: 18-26.

In some embodiments, the disclosure provides a composition comprising: (a) the fusion protein provided herein; and (b) a polynucleotide that forms a complex with the fusion protein and comprises (i) a guide sequence; and (ii) a template sequence for the reverse transcriptase, the DNA polymerase, or the DNA ligase.

In some embodiments, the polynucleotide comprises RNA. In some embodiments, the guide sequence comprises RNA and the template sequence comprises DNA. In some embodiments, the template sequence comprises an abasic site, a triethylene glycol (TEG) linker, or both. In some embodiments, the guide sequence is about 15 to about 20 nucleotides in length. In some embodiments, the polynucleotide further comprises a tracrRNA. In some embodiments, the composition comprises a second polynucleotide comprising a tracrRNA.

In some embodiments, the template sequence comprises a primer-binding sequence and a sequence of interest. In some embodiments, the primer-binding sequence and the sequence of interest comprise DNA. In some embodiments, the sequence of interest comprises DNA. In some embodiments, the template sequence is about 25 to about 10000 nucleotides in length. In some embodiments, the primer-binding sequence is about 4 to about 30 nucleotides in length. In some embodiments, the sequence of interest is about 5 nucleotides to about 9800 nucleotides in length.

In some embodiments, the polynucleotide comprises a spacer between the guide sequence and the template sequence. In some embodiments, the spacer is about 10 to about 200 nucleotides in length. In some embodiments, the spacer comprises a stop sequence for the reverse transcriptase or DNA polymerase. In some embodiments, the spacer comprises more than one stop sequence. In some embodiments, the stop sequence comprises a secondary structure. In some embodiments, the secondary structure is a hairpin loop.

In some embodiments, the disclosure provides a composition comprising: (a) the fusion protein provided herein; (b) a guide polynucleotide that forms a complex with the fusion protein and comprises a guide sequence; and (c) a template polynucleotide comprising a template sequence for the reverse transcriptase, the DNA polymerase, or the DNA ligase.

In some embodiments, the guide polynucleotide is RNA. In some embodiments, the template polynucleotide comprises RNA. In some embodiments, the template sequence comprises DNA. In some embodiments, the template sequence comprises an abasic site, a triethylene glycol (TEG) linker, or both. In some embodiments, the guide sequence is about 15 to about 20 nucleotides in length. In some embodiments, the guide polynucleotide further comprises a tracrRNA. In some embodiments, the composition further comprises a third polynucleotide comprising a tracrRNA.

In some embodiments, the template sequence is about 25 to about 10000 nucleotides in length. In some embodiments, the template sequence comprises a sequence of interest. In some embodiments, the sequence of interest is about 5 nucleotides to about 9800 nucleotides in length. In some embodiments, the sequence of interest comprises DNA.

In some embodiments, the template polynucleotide further comprises a primer-binding sequence. In some embodiments, the primer-binding sequence is about 10 to about 20 nucleotides in length. In some embodiments, the primer-binding sequence and the sequence of interest comprise DNA.

In some embodiments, the template polynucleotide further comprises a stop sequence for the reverse transcriptase or DNA polymerase. In some embodiments, the template polynucleotide comprises more than one stop sequence. In some embodiments, the stop sequence comprises a secondary structure. In some embodiments, the secondary structure is a hairpin loop.

In some embodiments, the template polynucleotide comprises an adeno-associated virus (AAV) vector comprising a sequence of interest.

In some embodiments, the disclosure provides a polynucleotide encoding the fusion protein provided herein. In some embodiments, the disclosure provides a vector comprising the polynucleotide encoding the fusion protein provided herein.

In some embodiments, the disclosure provides a cell comprising the fusion protein provided herein. In some embodiments, the disclosure provides a cell comprising the polynucleotide encoding the fusion protein provided herein, or the vector provided herein.

In some embodiments, the disclosure provides a cell comprising the composition provided herein.

In some embodiments, the disclosure provides a method of providing a site-specific modification at a target sequence in a target polynucleotide, the method comprising contacting the target polynucleotide with the composition provided herein.

In some embodiments, the target polynucleotide is DNA. In some embodiments, the guide sequence is capable of hybridizing to the target sequence. In some embodiments, the contacting is performed under conditions sufficient for the Cas nuclease to generate a double-stranded polynucleotide cleavage at the target sequence.

In some embodiments, the template sequence comprises a sequence of interest. In some embodiments, the template sequence comprises a primer-binding sequence capable of hybridizing to the target sequence.

In some embodiments, the contacting is performed under conditions sufficient for the reverse transcriptase to transcribe a complementary strand of the sequence of interest. In some embodiments, the method further comprises cleaving the template sequence to generate a double-stranded sequence comprising the sequence of interest. In some embodiments, the cleaving is performed by RNase H.

In some embodiments, the contacting is performed under conditions sufficient for the DNA polymerase to generate a double-stranded sequence comprising the sequence of interest. In some embodiments, the contacting is performed under conditions sufficient for the DNA ligase to ligate the sequence of interest to the cleaved target sequence.

In some embodiments, the double-stranded sequence comprising the sequence of interest is inserted into the cleaved target sequence by non-homologous end joining (NHEJ). In some embodiments, the double-stranded sequence comprising the sequence of interest is inserted into the cleaved target sequence by a DNA ligase.

In some embodiments, the method further comprises generating a second double-stranded polynucleotide cleavage at a second target sequence in the target polynucleotide. In some embodiments, the sequence of interest replaces a sequence of the target polynucleotide between the target sequence and the second target sequence.

In some embodiments, the disclosure provides a kit comprising the fusion protein provided herein.

In some embodiments, the kit further comprises a polynucleotide that forms a complex with the fusion protein and/or a vector for expressing the polynucleotide. In some embodiments, the kit further comprises a template polynucleotide comprising a template sequence for the reverse transcriptase, the DNA polymerase, or the DNA ligase and/or a vector for expressing the template polynucleotide. In some embodiments, the kit further comprises a polynucleotide comprising a tracrRNA. In some embodiments, the kit further comprises RNase H.

In some embodiments a Cas9-RT fusion is used with pegRNA and DNAPK inhibitor to increase gene editing efficiency

### BRIEF DESCRIPTION OF THE DRAWINGS

FIGS. 1A-1D illustrate an exemplary method described in embodiments herein. FIGS. 1A and 1B show a Cas9 fused to an “NHEJ-promoting domain,” e.g., a reverse transcriptase, DNA polymerase, or DNA ligase, the fusion protein termed PRimed INSertion (PRINS). In FIG. 1A, the “SPRINgRNA” (single primed insertion guide RNA) comprises an sequence of interest (“ins”) and a primer-binding site (PBS). In FIG. 1B, the fusion protein further comprises a DNA- or RNA-binding domain (e.g., MCP2, ZF, TALE, FBP, Pumilio, HUH, or SNAP), and the sequence of interest with the PBS is provided as separate polynucleotide. FIG. 1C shows the mechanism of action of the PRINS complex depicted in FIG. 1A. The Cas9 nuclease generates a double-stranded cleavage at the target polynucleotide. The template sequence in the Cas9 complex containing the PBS and sequence of interest is used to generate a double-stranded insert sequence comprising a copy of the sequence of interest. The double stranded insert sequence generated can then be ligated by NHEJ to the cleaved target polynucleotide. FIG. 1D shows a further embodiment for combining insertion and deletion. The Cas9 nuclease generates a double-stranded break at the target polynucleotide. The template sequence in the Cas9 complex containing the PBS and sequence of interest is used to generate a double-stranded insert sequence comprising a copy of the sequence of interest. The double stranded insert sequence generated can then be ligated by NHEJ to another break generated downstream by a second CRISPR/Cas complex. The sequence between the two CRISPR/Cas complexes is replaced by the sequence of interest.

FIGS. 2A-2E illustrate an exemplary method described in embodiments herein. FIG. 2A shows a Cas9-RT fusion protein (PRINS) with a guide RNA containing an insertion sequence (gRNA) generating a double-stranded break in a target sequence. The PRINS binds the gRNA for extension. FIG. 2B shows the result of the extension, with the extended sequence indicated by the dashed line. FIG. 2C shows the generation of a double-stranded break in the extended sequence, e.g., by RNase H. FIG. 2D shows the integration of the extended sequence into the cleaved target sequence by NHEJ. FIG. 2E shows the inserted sequence.

FIGS. 3A and 3B relate to Example 1 and show a comparison of Cas9 editing (FIG. 3A) vs. PRINS editing (FIG. 3B) at an AAVS1 site. Relative editing frequency was determined by RIMA as described in Example 1. Insertions are indicated by ovals. FIG. 3B shows that PRINS facilitates the template insertions of the sequence AAGATG, and PRINS promotes insertions over Cas9. All insertions are derived from the original sequence AAGATG.

FIG. 4 illustrates an exemplary method described in embodiments herein. A Cas nuclease is guided to a target sequence by the gRNA and generates a double-stranded DNA break. The template sequence comprises a primer-binding sequence that hybridizes with the cleaved DNA, which serves as a primer, and a sequence of interest. A reverse transcriptase, e.g., fused to the Cas9 nuclease, synthesizes the first cDNA from the primer. A DNA strand complementary to the first cDNA is generated by a polymerase, e.g., DNA polymerase. The first cDNA and the DNA strand complementary to the first cDNA hybridize to generate a double-stranded sequence, which can be inserted into the cleaved DNA by a DNA repair pathway, e.g., NHEJ.

FIGS. 5A-5D relate to Example 2 and show a comparison of Prime Editing, utilizing a prime editing guide RNA (pegRNA) (as described by Anzalone et al., Nature 576: 149-157 (2019)) vs. PRINS editing, utilizing a single primed insertion guide RNA (springRNA) at an AAVS1 site to insert the AAGATG sequence. Relative editing frequency was determined by Fragment analysis as described herein. Comparison of FIG. 5A (PRINS) to FIG. 5B (Prime Editing) shows that PRINS is more efficient than Prime Editing. FIGS. 5C and 5D demonstrate the NHEJ dependency of PRINS. FIGS. 5C and 5D show a comparison of PRINS (FIG. 5C) and Prime Editing (FIG. 5D) insertion frequency in the presence of a DNA-dependent protein kinase inhibitor, which is involved in NHEJ.

FIG. 6 relates to Example 3 and shows the effect of using pegRNA and springRNA with PRINS at an AAVS1 site to insert the AAGATG sequence. Relative editing frequency was determined by Fragment analysis as described herein. As shown in FIG. 6, pegRNA and springRNA can promote DNA insertion by PRINS either by a pathway similar to prime editing or by a pathway similar to PRINS (primed editing insertion).

FIG. 7 relates to Example 4 and shows the effect of using PRINS editing or prime editing, in the presence of absence of a DNA-dependent kinase (DNA-PK) inhibitor AZD7648. Specific integration was determined by NGS Amplicon-Seq as described herein. Bar graphs represent the average of n=2 with standard deviation. The bars labeled as “#1” or “#2” refer to different springRNA (for PRINS editing) or different pegRNA (for prime editing).

FIGS. 8-12 relate to Example 5. FIG. 8 shows a summary of the editing efficiency when using Cas9+RT (“PE0”) fusion, Cas9+DNA Polymerase D (“PE0 PolD”) fusion, Cas9+Phi29 DNA polymerase (“PE0 Phi”) fusion, or a Cas9 control, using either a DNA template sequence (“DNA tail”) containing springRNA or RNA template sequence (“RNA tail”) containing springRNA as described herein.

FIG. 9 shows the editing patterns using the Cas9+RT (“PE0”) fusion protein with three different guide RNAs, one containing an RNA tail (“123RNA MS”) and two containing DNA tails (“123DNA” and “123DNA PS”) as described herein. The top, middle, and bottom panels in FIG. 9 indicate the editing patterns of PE0 using 123RNA MS tail, 123DNA tail, or 123DNA PS tail, respectively.

FIG. 10 shows the editing patterns using the Cas9+DNA Polymerase D (“PE0 PolD”) fusion protein with three different guide RNAs, one containing an RNA tail (“123RNA MS”) and two containing DNA tails (“123DNA” and “123DNA PS”) as described herein. The top, middle, and bottom panels in FIG. 10 indicate the editing patterns of PE0 PolD using 123RNA MS tail, 123DNA tail, or 123DNA PS tail, respectively.

FIG. 11 shows the editing patterns using the Cas9+Phi29 DNA polymerase (“PE0 Phi”) fusion protein with three different guide RNAs, one containing an RNA tail (“123RNA MS”) and two containing DNA tails (“123DNA” and “123DNA PS”) as described herein. The top, middle, and bottom panels in FIG. 11 indicate the editing patterns of PE0 Phi using 123RNA MS tail, 123DNA tail, or 123DNA PS tail, respectively.

FIG. 12 shows the editing patterns using Cas9 with three different guide RNAs, one containing an RNA tail (“123RNA MS”) and two containing DNA tails (“123DNA” and “123DNA PS”) as described herein. The top, middle, and bottom panels in FIG. 12 indicate the editing patterns of Cas9 using 123RNA MS tail, 123DNA tail, or 123DNA PS tail, respectively.

FIGS. 13, 14A, and 14B relate to Example 6. FIG. 13 shows exemplary guide RNA designs for PRINS editing (labeled “PRINS #1” and “PRINS #2”) and prime editing (labeled “PE #1” and “PE #2”). As shown in FIG. 13, the prime editing guide RNA includes an additional 3′ homology region.

FIGS. 14A and 14B show the effect of using the different guide RNAs shown in FIG. 13 with PRINS editing or prime editing, and in the presence or absence of the DNA-PK inhibitor AZA7648. Specific integration was determined by NGS Amplicon-Seq as described herein. Bar graphs represent the average of n=2 with standard deviation.

FIGS. 15-16 relate to Example 7. FIG. 15 illustrates an exemplary schematic of the diphtheria toxin selection system described herein. As shown in FIG. 15, an intron of HbEGF, the DT receptor, was selected as the PRINS editing or Cas9 editing target. Only a bi-allelic large deletion will provide the cell with DT resistance.

FIG. 16 shows microscopy images of the cells transfected with a Cas9-RT fusion (PRINS editing, “PE0”), Cas9, or Cas9 nickase-RT fusion (prime editing, “PE2”) and three different guide RNAs. Positive control shows cells transfected with a Cas9 targeting HbEGF.

FIGS. 17-18 relate to Example 8. FIG. 17 shows an exemplary schematic of two Cas9+RT fusion proteins containing an MCP domain, either in between the Cas9 and RT (“PRINS_MS2_v1”) or downstream of the RT (“PRINS_MS2_v2”), as described herein. Three different polynucleotide systems were tested: (1) guide RNA and template polynucleotide for reverse transcriptase fused to MS2 aptamer as separate polynucleotides; (2) control, non-targeting guide RNA; and (3) guide RNA fused to reverse transcriptase template.

FIG. 18 shows the editing efficiency of PRINS editing for inserting the desired sequence AAGATG, using the Cas9+RT+MCP fusion proteins with the three different polynucleotide systems described in FIG. 17.

FIG. 19 relates to Example 9 and shows an exemplary guide RNA for Cas12 and targeting EXM1.

FIG. 20 relates to Example 10 and shows the results of PRINS editing by Cas9-DNA polymerase fusion proteins. The frequency of insertion of the springRNA insert sequence was analyzed in cells transfected with Cas9, Cas9-RT (“PE0”), or Cas9 fused to various DNA polymerases: Klenow fragment without 3′→5′ exonuclease activity (“Cas9-Klenow exo-”), Klenow fragment with 3′→5′ exonuclease activity (“Cas9-Klenow exo+”), or REV3 polymerase (“Cas9-REV3”). Each circle represents the frequency of the exact insert for each independent transfection. The dotted line represents the mean value of insertions by Cas9 only (i.e., background value), and the difference from the background for each tested condition was calculated by multiple comparison ANOVA (Brown-Forsythe and Welch adjustments). Mean and standard deviation of 10 to 15 measurements are represented as whisker plots. ***: p<0.0005; ****: p<0.0001.

FIGS. 21A-21C relate to Example 11 and show the results of PRINS editing by Cas9-DNA polymerase fusion proteins with chimeric springRNAs. Co-transfection of Cas9-DNA polymerase with chimeric springRNA with DNA and RNA insert sequence and PBS (“DiHP”) or springRNA with DNA insert sequence (“DiRP”) increases overall insertion efficiency, as shown in FIG. 27A, and increases the frequency of inserting the desired sequence, as shown in FIG. 27B. In FIGS. 27A and 27B, each symbol (circle, square, or hexagon) represents editing observed per sample. Circles represent springRNA, squares represent DiHP, and hexagons represent DiRP. Mean and standard deviation are represented by whisker plots. FIG. 27C shows the representative editing patterns of Cas9, PE0, and Cas9-DNA polymerase fusion proteins with springRNA, DiHP, and DiRP. In FIG. 27C, insertions are represented by shaded rectangles with the specified sequence, and deletions are represented by connecting lines.

FIG. 22 relates to Example 12 and shows the results of PRINS-editing by Cas9-RT using springRNA with modifications (abasic site or TEG linker). Co-transfection of Cas9-RT with modified springRNA increased the frequency of insertions with the desired length and therefore led to more precise modifications.

FIGS. 23A-23B relate to Example 13. FIG. 23A shows an electrogram of the AAVS1 locus after amplification with fluorescently-labeled PCR primers and resolution by capillary electrophoresis, after PRINS editing with PE0 (top panel) and Cas9 and RT expressed separately (bottom panel). The asterisk depicts DNA products corresponding to the wild-type sequence, and large molecules with 6 bp insertions correspond to PRINS-edited sequences. FIG. 23B shows the results of PRINS editing with Cas9, PE0, Cas9 and RT expressed separately, and Cas9-LigD and RT expressed separately. Co-expression of Cas9-LigD and RT improved insertion of the desired sequence as compared with co-expression of Cas9 and RT. Circles represent individual editing measurement of >4 biological replicates. Mean and standard deviation are represented by crossbar and whisker plots. Statistical difference was calculated by ANOVA (****: p<0.0001).

FIGS. 24A-24B relate to Example 14 and show the results of PRINS editing efficiency with or without mismatches in the springRNA PBS. FIG. 24A shows that PRINS editing using springRNA without any nucleobase mismatches had a relative insertion frequency of 37.13% for a 6-bp insertion sequence. FIG. 24B shows that PRINS editing using springRNA with a 2-bp nucleobase mismatch at the 3′ end of the PBS had a relative insertion frequency of 59.59% for a 4-nt insertion sequence (original 6-bp sequence minus the 2-bp mismatch).

FIG. 25 relates to Example 15 and shows the results of PRINS editing in cells that were partially deficient in one of the following DNA repair genes: PRKDC (also known as DNAPK), LIG4, TP53BP1, PARP1, POLQ, LIG3, and ATM. Experiments were performed in triplicate in the presence of DMSO control (“d”) or a DNAPK inhibitor (“i”). The left panel shows experiments with Cas9-RT fusion (“PE0”) and springRNA. The right panel shows experiments with PE0 and pegRNA.

FIGS. 26A-26B relate to Example 16. SEQ ID NO:29 in FIGS. 26A-26B show the springRNA containing the tracrRNA scaffold for MHCas9, 6-bp insert sequence, and PBS. FIG. 26A shows the most efficient PRINS editing events by MHCas9-RT. FIG. 26B shows the ten most frequent PRINS editing events by MHCas9-RT, indicating that the RT is mediating not only template insertions but also extended the overhang sequences (CCC) generated by the MHCas9, as indicated by the three most frequent editing events.

FIGS. 27A-27B relate to Example 17 and show the results of targeted substitution/insertions and deletions by Cas9-RT with pegRNA. FIG. 27A shows the frequency of A to G substitutions at the AAVS1 locus with DMSO or DNAPK inhibitor (DNAPKi). FIG. 27B shows the frequency of 1 nucleotide deletion at the AAVS1 locus with DMSO or DNAPKi.

### DETAILED DESCRIPTION OF THE INVENTION

The present disclosure relates to improved CRISPR systems and components thereof, and methods of using the same. In general, a CRISPR system, e.g., a CRISPR/Cas system, includes elements that promote the formation of a CRISPR complex, such as a guide polynucleotide and a Cas protein, at the site of a target polynucleotide, e.g., a target DNA sequence. In naturally-occurring CRISPR systems (e.g., the bacterial immunity CRISPR/Cas9 system), foreign DNA is incorporated into CRISPR arrays, which then produce CRISPR-RNAs (crRNA). The crRNA includes protospacer regions complementary to the foreign DNA site and hybridizes with trans-activating CRISPR-RNA (tracrRNA), which is also encoded by the CRISPR system. The tracrRNA forms secondary structures, e.g., stem loops, and is capable of binding to Cas9 protein. The crRNA/tracrRNA hybrid associates with Cas9, and the crRNA/tracrRNA/Cas9 complex recognizes and cleaves foreign DNA bearing the protospacer sequences, thereby conferring immunity against the invading virus or plasmid.

Since its original discovery, extensive research focused on potential applications of the CRISPR system in genetic engineering, including gene editing (see, e.g., Jinek et al., Science 337(6096):816-821 (2012); Cong et al., Science 339(6121):819-823 (2013); and Mali et al., Science 339(6121):823-826 (2013)). The CRISPR/Cas system, utilizing components of the naturally-occurring CRISPR systems described herein, has been used for site-specific genome modifications, e.g., gene editing, in a wide range of organisms and cell lines. In addition to gene editing, the CRISPR system has a multitude of other applications, including regulating gene expression, genetic circuit construction, functional genomics, etc. (reviewed in Sander and Joung, Nat Biotechnol 32:347-355 (2014)).

Unless otherwise defined herein, scientific and technical terms used in the present disclosure shall have the meanings that are commonly understood by one of ordinary skill in the art. Further, unless otherwise required by context, singular terms shall include pluralities and plural terms shall include the singular. As used herein, “a” or “an” may mean one or more. As used herein, when used in conjunction with the word “comprising,” the words “a” or “an” may mean one or more than one. As used herein, “another” or “a further” may mean at least a second or more.

A nucleic acid molecule is “hybridizable” or “hybridized” to another nucleic acid molecule, such as a cDNA, genomic DNA, or RNA, when a single stranded form of the nucleic acid molecule can anneal to the other nucleic acid molecule under the appropriate conditions of temperature and solution ionic strength. Hybridization and washing conditions are known and exemplified in Sambrook et al., Molecular Cloning: A Laboratory Manual, Second Edition, Cold Spring Harbor Laboratory Press, Cold Spring Harbor (1989), particularly Chapter 11 and Table 11.1 therein. The conditions of temperature and ionic strength determine the stringency of the hybridization. The stringency of the hybridization conditions can be selected to provide selective formation or maintenance of a desired hybridization product of two complementary nucleic acid polynucleotides, in the presence of other potentially cross-reacting or interfering polynucleotides. Stringent conditions are sequence-dependent; typically, longer complementary sequences specifically hybridize at higher temperatures than shorter complementary sequences. Generally, stringent hybridization conditions are between about 5° C. to about 10° C. lower than the thermal melting point (Tₘ) (i.e., the temperature at which 50% of the sequences hybridize to a substantially complementary sequence) for a specific polynucleotide at a defined ionic strength, concentration of chemical denaturants, pH, and concentration of the hybridization partners. Generally, nucleotide sequences having a higher percentage of G and C bases hybridize under more stringent conditions than nucleotide sequences having a lower percentage of G and C bases. Generally, stringency can be increased by increasing temperature, increasing pH, decreasing ionic strength, and/or increasing the concentration of chemical nucleic acid denaturants (such as formamide, dimethylformamide, dimethylsulfoxide, ethylene glycol, propylene glycol and ethylene carbonate). Stringent hybridization conditions typically include salt concentrations or ionic strength of less than about 1 M, 500 mM, 200 mM, 100 mM or 50 mM; hybridization temperatures above about 20° C., 30° C., 40° C., 60° C. or 80° C.; and chemical denaturant concentrations above about 10%, 20%, 30% 40% or 50%. Because many factors can affect the stringency of hybridization, the combination of parameters may be more significant than the absolute value of any parameter alone.

An exemplary low stringency hybridization condition, for example, corresponding to a Tm of 55° C., includes 5× saline-sodium citrate buffer (SSC), 0.1% SDS, 0.25% milk, and no formamide; or 30% formamide, 5×SSC, and 0.5% SDS. An exemplary moderate stringency hybridization condition corresponding to a higher T. of between about 55° C. and about 65° C., includes 40% formamide and 5× or 6×SCC. An exemplary high stringency hybridization condition corresponding to the highest Tm of greater than 65° C., includes 50% formamide and 5' or 6×SCC.

Further exemplary hybridization conditions include buffered solutions (for example, phosphate, Tris, or HEPES buffered solutions, having between around 20 mM and 200 mM of the buffering component) at pH between around 6.5 to 8.5, and having an ionic strength between about 20 mM and 200 mM, at a temperature between about 15° C. to 40° C. For example, the buffer may include a salt at a concentration of from about 10 mM to about 1 M, from about 20 mM to about 500 mM, from about 30 mM to about 100 mM, from about 40 mM to about 80 mM, or about 50 mM. Exemplary salts include NaCl, KCl, (NH₄)₂SO₄, Na₂SO₄, and CH₃COONH₄.

The term “complementary” is used to describe the relationship between nucleotide bases that are capable of hybridizing to one another. For example, with respect to DNA, adenosine is complementary to thymine and cytosine is complementary to guanine. Accordingly, the present disclosure also includes isolated nucleic acid fragments that are complementary to the complete sequences as disclosed or used herein as well as those substantially similar nucleic acid sequences.

The term “homologous recombination” refers to the insertion of a foreign polynucleotide (e.g., DNA) into another nucleic acid (e.g., DNA) molecule, e.g., insertion of a vector in a chromosome. In some cases, the vector targets a specific chromosomal site for homologous recombination. For specific homologous recombination, the vector typically contains sufficiently long regions of homology to sequences of the chromosome to allow complementary binding and incorporation of the vector into the chromosome. Longer regions of homology and greater degrees of sequence similarity may increase the efficiency of homologous recombination. In some embodiments, the fusion proteins or compositions described herein facilitate homologous recombination by generating breaks, e.g., double-stranded breaks in a nucleic acid sequence.

As used herein, the term “operably linked” means that a polynucleotide of interest, e.g., the polynucleotide encoding a nuclease, is linked to the regulatory element in a manner that allows for expression of the polynucleotide. In some embodiments, the regulatory element is a promoter. In some embodiments, polynucleotide expressing the polypeptide of interest is operably linked to a promoter on an expression vector.

A “vector” is any means for the cloning of and/or transfer of a nucleic acid into a host cell. A vector may be a replicon to which another DNA segment may be attached so as to bring about the replication of the attached segment. A “replicon” is any genetic element (e.g., plasmid, phage, cosmid, chromosome, virus) that functions as an autonomous unit of DNA replication in vivo, i.e., capable of replication under its own control. In some embodiments, the vector is an episomal vector, which is removed/lost from a population of cells after a number of cellular generations, e.g., by asymmetric partitioning. The term “vector” includes both viral and non-viral means for introducing the nucleic acid into a cell in vitro, ex vivo, or in vivo. A large number of vectors known in the art may be used to manipulate nucleic acids, incorporate response elements and promoters into genes, etc. A vector may include one or more regulatory regions, and/or selectable markers useful in selecting, measuring, and monitoring nucleic acid transfer results (transfer to which tissues, duration of expression, etc.).

Possible vectors include, for example, plasmids or modified viruses including, for example, bacteriophages such as lambda derivatives, or plasmids such as PBR322 or pUC plasmid derivatives, or the Bluescript vector. For example, the insertion of the DNA fragments corresponding to response elements and promoters into a suitable vector can be accomplished by ligating the appropriate DNA fragments into a chosen vector that has complementary cohesive termini. Alternatively, the ends of the DNA molecules may be enzymatically modified, or any site may be produced by ligating polynucleotides (linkers) into the DNA termini. Such vectors may be engineered to contain selectable marker genes that provide for the selection of cells that have incorporated the marker into the cellular genome. Such markers allow identification and/or selection of host cells that incorporate and express the proteins encoded by the marker.

Viral vectors, and particularly retroviral vectors, have been used in a wide variety of gene delivery applications in cells, as well as living animal subjects. Viral vectors that can be used include, but are not limited, to retrovirus, adenovirus, adeno-associated virus, pox, baculovirus, vaccinia, herpes simplex, Epstein-Barr, adenovirus, geminivirus, and caulimovirus vectors. In some embodiments, a viral vector is utilized to provide the polynucleotides described herein. In some embodiments, a viral vector is utilized to provide a polynucleotide coding for a polypeptide described herein.

Vectors may be introduced into the desired host cells by known methods, including, but not limited to, transfection, transduction, cell fusion, and lipofection. Vectors can include various regulatory elements including promoters. In some embodiments, vector designs can be based on constructs designed by Mali et al., Nat Methods 10: 957-63 (2013).

Methods known in the art may be used to propagate polynucleotides and/or vectors provided herein. Once a suitable host system and growth conditions are established, recombinant expression vectors can be propagated and prepared in quantity. As described herein, the expression vectors which can be used include, but are not limited to, the following vectors or their derivatives: human or animal viruses such as vaccinia virus or adenovirus; insect viruses such as baculovirus; yeast vectors; bacteriophage vectors (e.g., lambda), and plasmid and cosmid DNA vectors.

The term “plasmid” refers to an extra chromosomal element often carrying a gene that is not part of the central metabolism of the cell, and usually in the form of circular double-stranded DNA molecules. Such elements may be autonomously replicating sequences, genome integrating sequences, phage or nucleotide sequences, linear, circular, or supercoiled, of a single- or double-stranded DNA or RNA, derived from any source, in which a number of polynucleotides have been joined or recombined into a unique construction which is capable of introducing a promoter fragment and DNA sequence for a selected gene product along with appropriate 3′ untranslated sequence into a cell. In some embodiments, a plasmid is utilized to provide the polynucleotides described herein. In some embodiments, a plasmid is utilized to provide a polynucleotide coding for a polypeptide described herein.

The term “transfection” as used herein means the introduction of an exogenous nucleic acid molecule, including a vector, into a cell. A “transfected” cell includes an exogenous nucleic acid molecule inside the cell and a “transformed” cell is one in which the exogenous nucleic acid molecule within the cell induces a phenotypic change in the cell. The transfected nucleic acid molecule can be integrated into the host cell's genomic DNA and/or can be maintained by the cell, temporarily or for a prolonged period of time, extra-chromosomally. Host cells or organisms that express exogenous nucleic acid molecules or fragments are referred to herein as “recombinant,” “transformed,” or “transgenic” organisms. In some embodiments, the present disclosure provides a host cell including any of the expression vectors described herein, e.g., an expression vector including a polynucleotide encoding a nuclease, a fusion protein, or a variant thereof

The term “host cell” refers to a cell into which a recombinant expression vector has been introduced, or “host cell” may also refer to the progeny of such a cell. Because modifications may occur in succeeding generations, for example, due to mutation or environmental influences, the progeny may not be identical to the parent cell, but are still included within the scope of the term “host cell.”

The terms “peptide,” “polypeptide,” and “protein” are used interchangeably herein, and refer to a polymeric form of amino acids of any length, which can include coded and non-coded amino acids, chemically or biochemically modified or derivatized amino acids, and polypeptides having modified peptide backbones.

The start of the protein or polypeptide is known as the “N-terminus” (and also referred to as the amino-terminus, NH₂-terminus, N-terminal end or amine-terminus), referring to the free amine (—NH₂) group of the first amino acid residue of the protein or polypeptide. The end of the protein or polypeptide is known as the “C-terminus” (and also referred to as the carboxy-terminus, carboxyl-terminus, C-terminal end, or COOH-terminus), referring to the free carboxyl group (—COOH) of the last amino acid residue of the protein or polypeptide.

An “amino acid” as used herein refers to a compound including both a carboxyl (—COOH) and amino (-NH2) group. “Amino acid” refers to both natural and unnatural, i.e., synthetic, amino acids. Natural amino acids, with their three-letter and single-letter abbreviations, include: alanine (Ala; A); arginine (Arg, R); asparagine (Asn; N); aspartic acid (Asp; D); cysteine (Cys; C); glutamine (Gln; Q); glutamic acid (Glu; E); glycine (Gly; G); histidine (His; H); isoleucine (Ile; I); leucine (Leu; L); lysine (Lys; K); methionine (Met; M); phenylalanine (Phe; F); proline (Pro; P); serine (Ser; S); threonine (Thr; T); tryptophan (Trp; W); tyrosine (Tyr; Y); and valine (Val; V). Unnatural or synthetic amino acids include a side chain that is distinct from the natural amino acids provided above and may include, e.g., fluorophores, post-translational modifications, metal ion chelators, photocaged and photocross-linking moieties, uniquely reactive functional groups, and NMR, IR, and x-ray crystallographic probes. Exemplary unnatural or synthetic amino acids are provided in, e.g., Mitra et al., Mater Methods 3:204 (2013) and Wals et al., Front Chem 2:15 (2014). Unnatural amino acids may also include naturally-occurring compounds that are not typically incorporated into a protein or polypeptide, such as, e.g., citrulline (Cit), selenocysteine (Sec), and pyrrolysine (Pyl).

An “amino acid substitution” refers to a polypeptide or protein including one or more substitutions of wild-type or naturally occurring amino acid with a different amino acid relative to the wild-type or naturally occurring amino acid at that amino acid residue. The substituted amino acid may be a synthetic or naturally occurring amino acid. In some embodiments, the substituted amino acid is a naturally occurring amino acid selected from the group consisting of: A, R, N, D, C, Q, E, G, H, I, L, K, M, F, P, S, T, W, Y, and V. In some embodiments, the substituted amino acid is an unnaturally or synthetic amino acid. Substitution mutants may be described using an abbreviated system. For example, a substitution mutation in which the fifth (5^{th}) amino acid residue is substituted may be abbreviated as “XSY,” wherein “X” is the wild-type or naturally occurring amino acid to be replaced, “5” is the amino acid residue position within the amino acid sequence of the protein or polypeptide, and “Y” is the substituted, or non-wild-type or non-naturally occurring, amino acid.

An “isolated” polypeptide, protein, peptide, or nucleic acid is a molecule that has been removed from its natural environment. It is also understood that “isolated” polypeptides, proteins, peptides, or nucleic acids may be formulated with excipients such as diluents or adjuvants and still be considered isolated. As used herein, “isolated” does not necessarily imply any particular level purity of the polypeptide, protein, peptide, or nucleic acid.

The term “recombinant” when used in reference to a nucleic acid molecule, peptide, polypeptide, or protein means of, or resulting from, a new combination of genetic material that is not known to exist in nature. A recombinant molecule can be produced by any of the techniques available in the field of recombinant technology, including, but not limited to, polymerase chain reaction (PCR), gene splicing (e.g., using restriction endonucleases), and solid-phase synthesis of nucleic acid molecules, peptides, or proteins.

The term “domain” when used in reference to a polypeptide or protein means a distinct functional and/or structural unit in a protein. Domains are sometimes responsible for a particular function or interaction, contributing to the overall role of a protein. Domains may exist in a variety of biological contexts. Similar domains may be found in proteins with different functions. Alternatively, domains with low sequence identity (i.e., less than about 50%, less than about 40%, less than about 30%, less than about 20%, less than about 10%, less than about 5%, or less than about 1% sequence identity) may have the same function.

The term “motif,” when used in reference to a polypeptide or protein, generally refers to a set of conserved amino acid residues, typically shorter than 20 amino acids in length, that may be important for protein function. Specific sequence motifs may mediate a common function, such as protein-binding or targeting to a particular subcellular location, in a variety of proteins. Examples of motifs include, but are not limited to, nuclear localization signals, microbody targeting motifs, motifs that prevent or facilitate secretion, and motifs that facilitate protein recognition and binding. Motif databases and/or motif searching tools are known in the field and include, for example, PROSITE (expasy.ch/sprot/prosite.html), Pfam (pfam.wustl.edu), PRINTS (biochem.ucl.ac.uk/bsm/dbbrowser/PRINTS/PRINTS.html), and Minimotif Miner.

An “engineered” protein, as used herein, means a protein that includes one or more modifications in a protein to achieve a desired property. Exemplary modifications include, but are not limited to, insertion, deletion, substitution, and/or fusion with another domain or protein. A “fusion protein” (also termed “chimeric protein”) is a protein comprising at least two domains, typically coded by two separate genes, that have been joined such that they are transcribed and translated as a single unit, thereby producing a single polypeptide having the functional properties of each of the domains. Engineered proteins of the present disclosure include nucleases and fusion proteins, e.g., of a Cas nuclease and a reverse transcriptase, a DNA polymerase, or a DNA ligase.

In some embodiments, engineered protein is generated from a wild-type protein. As used herein, a “wild-type” protein or nucleic acid is a naturally-occurring, unmodified protein or nucleic acid. For example, a wild-type Cas9 protein can be isolated from the organism Streptococcus pyogenes. Wild-type can be contrasted with “mutant,” which includes one or more modifications in the amino acid and/or nucleotide sequence of the protein or nucleic acid. In some embodiments, an engineered protein can have substantially the same activity as a wild-type protein, e.g., greater than about 80%, greater than about 85%, greater than about 90%, greater than about 95%, or greater than about 99% of the activity as a wild-type protein. In some embodiments, the Cas nuclease of the fusion protein described herein has substantially the same activity as a wild-type Cas nuclease.

As used herein, the terms “sequence similarity” or “% similarity” refers to the degree of identity or correspondence between nucleic acid sequences or amino acid sequences. In the context of polynucleotides, “sequence similarity” may refer to nucleic acid sequences wherein changes in one or more nucleotide bases results in substitution of one or more amino acids, but do not affect the functional properties of the protein encoded by the polynucleotide. “Sequence similarity” may also refer to modifications of the polynucleotide, such as deletion or insertion of one or more nucleotide bases, that do not substantially affect the functional properties of the resulting transcript. It is therefore understood that the present disclosure encompasses more than the specific exemplary sequences. Methods of making nucleotide base substitutions are known, as are methods of determining the retention of biological activity of the encoded polypeptide.

Moreover, the skilled artisan recognizes that similar polynucleotides encompassed by the present disclosure are also defined by their ability to hybridize, under stringent conditions, with the sequences exemplified herein. Similar polynucleotides of the present disclosure are about 70%, at least about 70%, about 75%, at least about 75%, about 80%, at least about 80%, about 85%, at least about 85%, about 90%, at least about 90%, about 95%, at least about 95%, about 99%, at least about 99%, or about 100% identical to the polynucleotides disclosed herein.

In the context of polypeptides, “sequence similarity” refers to two or more polypeptides wherein greater than about 40% of the amino acids are identical, or greater than about 60% of the amino acids are functionally identical. “Functionally identical” or “functionally similar” amino acids have chemically similar side chains. For example, amino acids can be grouped in the following manner according to functional similarity:
Positively-charged side chains: Arg, His, Lys;
Negatively-charged side chains: Asp, Glu;
Polar, uncharged side chains: Ser, Thr, Asn, Gln;
Hydrophobic side chains: Ala, Val, Ile, Leu, Met, Phe, Tyr, Trp;
Other: Cys, Gly, Pro.

In some embodiments, similar polypeptides of the present disclosure have about 40%, at least about 40%, about 45%, at least about 45%, about 50%, at least about 50%, about 55%, at least about 55%, about 60%, at least about 60%, about 65%, at least about 65%, about 70%, at least about 70%, about 75%, at least about 75%, about 80%, at least about 80%, about 85%, at least about 85%, about 90%, at least about 90%, about 95%, at least about 95%, about 97%, at least about 97%, about 98%, at least about 98%, about 99%, at least about 99%, or about 100% identical amino acids.

In some embodiments, similar polypeptides of the present disclosure have about 60%, at least about 60%, about 65%, at least about 65%, about 70%, at least about 70%, about 75%, at least about 75%, about 80%, at least about 80%, about 85%, at least about 85%, about 90%, at least about 90%, about 95%, at least about 95%, about 97%, at least about 97%, about 98%, at least about 98%, about 99%, at least about 99%, or about 100% functionally identical amino acids.

Sequence similarity can be determined by sequence alignment using methods known in the field, such as, for example, BLAST, MUSCLE, Clustal (including ClustalW and ClustalX), and T-Coffee (including variants such as, for example, M-Coffee, R-Coffee, and Expresso).

Percent identity of polynucleotides or polypeptides can be determined when the polynucleotide or polypeptide sequences are aligned over a specified comparison window. In some embodiments, only specific portions of two or more sequences are aligned to determine sequence identity. In some embodiments, only specific domains of two or more sequences are aligned to determine sequence similarity. A comparison window can be a segment of at least 10 to over 1000 residues, at least 20 to about 1000 residues, or at least 50 to 500 residues in which the sequences can be aligned and compared. Methods of alignment for determination of sequence identity are well-known and can be performed using publicly available databases such as BLAST. For example, in some embodiments, “percent identity” of two amino acid sequences is determined using the algorithm of Karlin and Altschul, Proc Nat Acad Sci USA 87:2264-2268 (1990), modified as in Karlin and Altschul, Proc Nat Acad Sci USA 90:5873-5877 (1993). Such algorithms are incorporated into BLAST programs, e.g., BLAST+ or the NBLAST and) (BLAST programs described in Altschul et al., J Mol Biol, 215: 403-410 (1990). BLAST protein searches can be performed with programs such as, e.g., the XBLAST program, score=50, wordlength=3 to obtain amino acid sequences homologous to the protein molecules of the disclosure. Where gaps exist between two sequences, Gapped BLAST can be utilized as described in Altschul et al., Nucleic Acids Res 25(17): 3389-3402 (1997). When utilizing BLAST and Gapped BLAST programs, the default parameters of the respective programs (e.g., XBLAST and NBLAST) can be used.

In some embodiments, a polypeptide or polynucleotide has 70%, at least 70%, 75%, at least 75%, 80%, at least 80%, 85%, at least 85%, 90%, at least 90%, 95%, at least 95%, 97%, at least 97%, 98%, at least 98%, 99%, or at least 99% or 100% sequence identity with a reference polypeptide or polynucleotide (or a fragment of the reference polypeptide or polynucleotide) provided herein. In some embodiments, a polypeptide or polynucleotide have about 70%, at least about 70%, about 75%, at least about 75%, about 80%, at least about 80%, about 85%, at least about 85%, about 90%, at least about 90%, about 95%, at least about 95%, about 97%, at least about 97%, about 98%, at least about 98%, about 99%, at least about 99% or about 100% sequence identity with a reference polypeptide or polynucleotide (or a fragment of the reference polypeptide or nucleic acid molecule) provided herein.

As used herein, a “complex” refers to a group of two or more associated polynucleotides and/or polypeptides. In the context of complex formation, the terms “associate” or “association” refers to molecules bound to one another through electrostatic, hydrophobic/hydrophilic, and/or hydrogen bonding interaction, without being covalently attached. A molecule that comprises different moieties covalently attached to one another is known. In some embodiments, a complex is formed when all the components of the complex are present together, i.e., a self-assembling complex. In some embodiments, a complex is formed through chemical interactions between different components of the complex such as, for example, hydrogen-bonding. In some embodiments, a polynucleotide, e.g., a RNA polynucleotide, forms a complex with a protein or polypeptide, e.g., a RNA-guided protein, through secondary structure recognition of the polynucleotide by the protein or polypeptide.

### Fusion Proteins

The fusion protein of the present disclosure provides improved gene editing efficiency compared with a wild-type Cas nuclease.

In some embodiments, the disclosure provides a fusion protein comprising: (i) a Cas nuclease and (ii) a reverse transcriptase, or a DNA polymerase, or a DNA ligase, wherein the Cas nuclease is capable of generating a double-stranded polynucleotide cleavage.

As described herein, fusion proteins typically include at least two domains having different functions. In some embodiments, the fusion protein comprises a Cas nuclease. In general, Cas nucleases are part of a CRISPR/Cas system. As described herein, CRISPR/Cas systems can be utilized for site-specific genome modifications. A CRISPR/Cas system can include a Cas nuclease and a guide polynucleotide (e.g., a guide RNA). In some embodiments, the guide polynucleotide comprises a polypeptide-binding segment, which binds and/or activates the Cas nuclease, and a guide sequence (e.g., crRNA), which hybridizes to a target sequence. As used herein, a “segment” refers to a part, section, or region of a molecule, e.g., a contiguous stretch of nucleotides of a guide polynucleotide molecule. The definition of “segment,” unless otherwise specifically defined, is not limited to a specific number of total base pairs. In some embodiments, the guide polynucleotide comprises a tracrRNA. In some embodiments, the guide polynucleotide does not comprise a tracrRNA, and the tracrRNA is provided as a separate polynucleotide in the CRISPR/Cas system. In some embodiments, the tracrRNA activates the Cas nuclease. In some embodiments, activation of the Cas nuclease initiates or increases its nuclease activity. In some embodiments, activation of the Cas nuclease comprises binding of the nuclease to a target sequence in a target polynucleotide.

CRISPR/Cas systems can be classified as Types Ito VI, based on the nuclease protein in the system. For example, Cas9 can be found in Type II systems, while Cas12 can be found in Type V systems. Each Type can be further divided into subtypes. For example, Type II can include subtypes II-A, II-B, and II-C, and Type V can include subtypes V-A and V-B. Classification of CRISPR/Cas systems and Cas nucleases is further discussed in, e.g., Makarova et al., Methods Mol Biol 1311:47-75 (2015); Makarova et al., The CRISPR Journal October 2018; 325-336; and Koonin et al., Phil Trans R Soc B 374:20180087 (2018). Cas nucleases described herein can encompass any Type or variant, unless otherwise specified.

In some embodiments, the Cas nuclease is capable of generating a double-stranded polynucleotide cleavage, e.g., a double-stranded DNA cleavage. In general, a Cas nuclease can include one or more nuclease domains, such as RuvC and HNH, and can cleave double-stranded DNA. In some embodiments, a Cas nuclease comprises a RuvC domain and an HNH domain, each of which cleaves one strand of double-stranded DNA. In some embodiments, the Cas nuclease generates blunt ends. In some embodiments, the RuvC and HNH of a Cas nuclease cleaves each DNA strand at the same position, thereby generating blunt ends. In some embodiments, the Cas nuclease generates cohesive ends. In some embodiments, the RuvC and HNH of a Cas nuclease cleaves each DNA strand at different positions (i.e., cut at an “offset”), thereby generating cohesive ends. As used herein, the terms “cohesive ends,” “staggered ends,” or “sticky ends” refer to a nucleic acid fragment with strands of unequal length. In contrast to “blunt ends,” cohesive ends are produced by a staggered cut on a double-stranded nucleic acid (e.g., DNA). A sticky or cohesive end has protruding singles strands with unpaired nucleotides, or “overhangs,” e.g., a 3′ or a 5′ overhang.

In some embodiments, the Cas nuclease is Cas9. Cas9 is found in Type II CRISPR/Cas systems as described herein. Exemplary Cas9 proteins include, but are not limited to, the Cas9 protein from Streptococcus pyogenes, Streptococcus thermophilus, Streptococcus mutans, Listeria innocua, Neisseria meningitidis, Staphylococcus aureus, Klebisella pneumoniae, and numerous other bacteria. Further exemplary Cas9 nucleases are described in, e.g., U.S. Pat. Nos. 8,771,945, 9,023,649, 10,000,772, and 10,407,697. In some embodiments, Cas9 refers to a polypeptide of SEQ ID NO: 1.

In some embodiments, the Cas9 is a Type IIB Cas9. In general, Type IIB Cas9 proteins are capable of generating cohesive ends, as described herein. Exemplary Type IIB Cas9 proteins include, but are not limited to, the Cas9 protein from Legionella pneumophila, Francisella novicida, Parasutterella excrementihominis, Sutterella wadsworthensis, Wolinella succinogenes, and numerous other bacteria. In some embodiments, the Type IIBCas9 is from the sequenced gut metagenome MI-10245_GL0161830.1 (MHCas9). Further Type IIB Cas9 proteins are described in, e.g., WO 2019/099943.

In some embodiments, the Cas9 comprises SEQ ID NO: 1. In some embodiments, the Cas9 comprises a polypeptide sequence having at least 70%, at least 75%, at least 80%, at least 85%, at least 90%, at least 95%, at least 96%, at least 97%, at least 98%, at least 99%, or about 100% sequence identity to SEQ ID NO: 1. In some embodiments, the disclosure provides for a polynucleotide which encodes a polypeptide having at least 70%, at least 75%, at least 80%, at least 85%, at least 90%, at least 95%, at least 96%, at least 97%, at least 98%, at least 99%, or about 100% sequence identity to SEQ ID NO: 1. In some embodiments, the Cas9 is encoded by a polynucleotide which has been codon optimized for expression in a host cell.

In some embodiments, the Cas nuclease is Cas12. Cas12 nucleases are sometimes known as “Cpfl” or “C2c1” nucleases and are found in Type V CRISPR/Cas systems as described herein. Cas12 nuclease are typically smaller than Cas9 nucleases and are capable of generating cohesive ends. Exemplary Cas12 proteins include, but are not limited to, the Cas12 protein from Francisella novicida, Acidaminococcus sp., Lachnospiraceae sp., Prevotella sp., and numerous other bacteria. Further Cas12 nuclease are described in, e.g., U.S. Pat. No. 9,580,701, US 2016/0208243, Zetsche et al., Cell 163(3):759-771 (2015), and Chen et al., Science 360:436-439 (2018).

In some embodiments, the Cas12 comprises SEQ ID NO: 29. In some embodiments, the Cas12 has at least 70%, at least 75%, at least 80%, at least 85%, at least 90%, at least 95%, at least 96%, at least 97%, at least 98%, at least 99%, or about 100% sequence identity to SEQ ID NO: 29. In some embodiments, the disclosure provides for a polynucleotide which encodes a polypeptide having at least 70%, at least 75%, at least 80%, at least 85%, at least 90%, at least 95%, at least 96%, at least 97%, at least 98%, at least 99%, or about 100% sequence identity to SEQ ID NO: 29. In some embodiments, the Cas12 is encoded by a polynucleotide which has been codon optimized for expression in a host cell.

In some embodiments, the Cas nuclease is Cas14. Cas14 nucleases, originally discovered in archaea, are small enzymes that typically target single-stranded DNA (ssDNA) and do not require a PAM sequence. Cas14 can be found in the DPANN superphylum of Archaea and are further described in, e.g., Harrington et al., Science 362:839-842 (2018) and US 2020/0087640.

In some embodiments, the Cas14 comprises SEQ ID NO: 30. In some embodiments, the Cas14 has at least 70%, at least 75%, at least 80%, at least 85%, at least 90%, at least 95%, at least 96%, at least 97%, at least 98%, at least 99%, or about 100% sequence identity to SEQ ID NO: 30. In some embodiments, the disclosure provides for a polynucleotide which encodes a polypeptide having at least 70%, at least 75%, at least 80%, at least 85%, at least 90%, at least 95%, at least 96%, at least 97%, at least 98%, at least 99%, or about 100% sequence identity to SEQ ID NO: 30. In some embodiments, the Cas14 is encoded by a polynucleotide which has been codon optimized for expression in a host cell.

In some embodiments, the fusion protein comprises a Cas nuclease and a reverse transcriptase, a DNA polymerase, a DNA ligase, or a combination thereof.

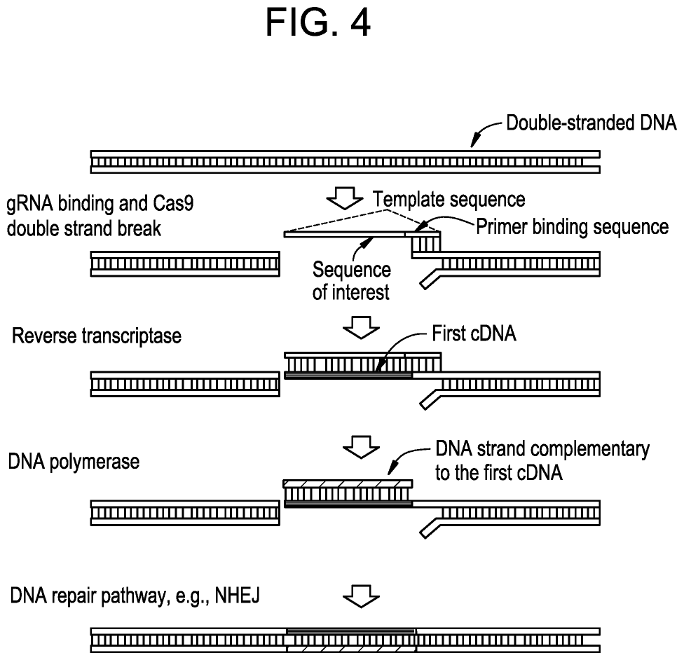

In some embodiments, the fusion protein comprises reverse transcriptase. Reverse transcriptase (sometimes abbreviated as RT) is an enzyme used to generate DNA (e.g., complementary DNA or cDNA) from an RNA template, a process called reverse transcription. A typical reverse transcription reaction is initiated with RNA template and a primer that binds to an end of the RNA template. In some embodiments, the reverse transcriptase binds to the primer (e.g., PBS) and synthesizes a strand of cDNA (e.g., based on the RNA template) in a process to provide a first cDNA. An exemplary, non-limiting, outline of the use of a Cas nuclease, reverse transcriptase, polymerase, and NHEJ to insert a sequence of interest is provided in FIG. 4. In some embodiments, an RNase, e.g., RNase H, removes the RNA template. In some embodiments, the reverse transcriptase comprises RNase activity, e.g., RNase H. In some embodiments, a DNA strand complementary to the first cDNA is then synthesized by DNA polymerase to generate a double-stranded sequence. In some embodiments, the reverse transcriptase comprises DNA polymerase activity. In some embodiments, DNA repair mechanisms, e.g., NHEJ, can be used to insert the double stranded sequence comprising the sequence of interest into the double stranded polynucleotide.

Exemplary reverse transcriptases include, but are not limited to, AMV reverse transcriptase, MMLV (M-MuLV) reverse transcriptase, R2 reverse transcriptase, and HIV reverse transcriptase. In some embodiments, the reverse transcriptase is MMLV reverse transcriptase or R2 reverse transcriptase. In some embodiments, the reverse transcriptase is capable of DNA polymerase activity.

In some embodiments, the Cas nuclease of the fusion protein generates a double-stranded polynucleotide cleavage at a target sequence in a target polynucleotide, e.g., a target DNA sequence. In some embodiments, one strand of the cleaved DNA serves as a primer for the reverse transcriptase of the fusion protein. In some embodiments, a template polynucleotide containing a template sequence for the reverse transcriptase is provided, and the reverse transcriptase generates a first cDNA. In some embodiments, the template sequence is RNA, and an RNase removes the template sequence. In some embodiments, the reverse transcriptase comprises RNase activity. In some embodiments, the template sequence is removed by a separate RNase. In some embodiments, the RNase is RNase H. In some embodiments, a DNA strand complementary to the first cDNA is generated by a DNA polymerase, e.g., a separate DNA polymerase or a reverse transcriptase having DNA polymerase activity. In some embodiments, the first cDNA and the DNA strand complementary to the first cDNA hybridize to form a double-stranded sequence. In some embodiments, the double-stranded sequence is capable of being inserted into the cleaved target sequence. In some embodiments, the double-stranded sequence is inserted into the cleaved target sequence by a DNA repair pathway. In some embodiments, the DNA repair pathway is non-homologous end joining (NHEJ), microhomology mediated end joining (MMEJ), homology directed repair (HDR), or a combination thereof. In some embodiments, the double-stranded sequence is inserted into the cleaved target sequence by ligation, e.g., using a DNA ligase.

In some embodiments, the reverse transcriptase comprises any one of SEQ ID NOS: 2-3. In some embodiments, the reverse transcriptase has at least 70%, at least 75%, at least 80%, at least 85%, at least 90%, at least 95%, at least 96%, at least 97%, at least 98%, at least 99%, or about 100% sequence identity to any one of SEQ ID NOS: 2-3. In some embodiments, the disclosure provides for a polynucleotide encoding a polynucleotide having at least 70%, at least 75%, at least 80%, at least 85%, at least 90%, at least 95%, at least 96%, at least 97%, at least 98%, at least 99%, or about 100% sequence identity to any one of SEQ ID NOS: 2-3. In some embodiments, the reverse transcriptase is encoded by a polynucleotide which has been codon optimized for expression in a host cell.

In some embodiments, the fusion protein comprises DNA polymerase. DNA polymerase is an enzyme that synthesizes DNA by adding nucleotides to an existing single DNA strand. In some embodiments, DNA polymerase generates a double-stranded sequence from a first synthesized strand generated by reverse transcriptase. In some embodiments, DNA polymerase generates double-stranded DNA from a single-stranded DNA template (ssDNA).

In some embodiments, the Cas nuclease of the fusion protein generates a double-stranded polynucleotide cleavage at a target sequence in a target polynucleotide, e.g., a target DNA sequence. In some embodiments, a template polynucleotide, e.g., an ssDNA template, is provided, and the DNA polymerase of the fusion protein generates a double-stranded sequence from the ssDNA template. In some embodiments, the double-stranded sequence is capable of being inserted into the cleaved target sequence. In some embodiments, the double-stranded sequence is inserted into the cleaved target sequence by a DNA repair pathway. In some embodiments, the DNA repair pathway is non-homologous end joining (NHEJ), microhomology mediated end joining (MMEJ), or homology directed repair (HDR). In some embodiments, the double-stranded sequence is inserted into the cleaved target sequence by ligation, e.g., using a DNA ligase.

Exemplary DNA polymerases include, but are not limited to, DNA Polymerase (Pol) I, II, III, IV, and V; DNA polymerase (Pol) α, β, λ, γ, σ, μ, δ, ε, η, ι, κ, ζ, θ, Rev1, and Rev3; isothermal DNA polymerases including, e.g., Bst, T4, and Φ29 (phi29) DNA polymerase; and thermostable DNA polymerases including, e.g., Taq, Pfu, KOD, Tth, and Pwo DNA polymerase. In some embodiments, the DNA polymerase is part of a DNA repair pathway. In some embodiments, the DNA repair pathway DNA polymerase is Pol β, Pol γ, Pol σ, or Pol μ. In some embodiments, the DNA polymerase is Rev3. DNA repair pathways are further described herein. In some embodiments, the DNA polymerase has high processivity, i.e., the DNA polymerase can process a large number of nucleotides in a single binding event. In some embodiments, the high processivity DNA polymerase is capable of greater than 100 bp, greater than 200 bp, greater than 300 bp, greater than 400 bp, greater than 500 bp, greater than 600 bp, greater than 700 bp, greater than 800 bp, greater than 1 kb, greater than 5 kb, greater than 10 kb, greater than 50 kb, or greater than 100 kb per binding event. In some embodiments, a high processivity DNA polymerase is advantageous for synthesizing long templates and sequences with secondary structures such as high GC content. In some embodiments, the high processivity DNA polymerase is Pol α, Pol δ, Pol ε, or Φ29 DNA polymerase. In some embodiments, the DNA polymerase is phi29 DNA polymerase, T4 DNA polymerase, DNA polymerase μ (mu), DNA polymerase δ (delta), or DNA polymerase ε (epsilon). In some embodiments, the DNA polymerase of the fusion protein comprises a catalytically active fragment or truncation of a DNA polymerase. As used herein, a “catalytically active” fragment, truncation, or domain of an enzyme means that the fragment or truncation has substantially the same activity as the full-length or wild-type form of the enzyme (e.g., DNA polymerase). In some embodiments, a catalytically active fragment, truncation, or domain of an enzyme herein has about 50%, about 60%, about 70%, about 80%, about 90%, about 100%, about 110%, about 120%, about 130%, about 140%, about 150%, about 160%, about 170%, about 180%, about 190%, about 200%, or greater than 200% of the activity of full-length or wild-type enzyme (e.g., DNA polymerase). In some embodiments, a catalytically active truncation, fragment, or domain of an enzyme herein has one or more improved properties as compared to the full-length or wild-type enzyme (e.g., DNA polymerase), such as improved stability and/or processivity. In some embodiments, the DNA polymerase is a Klenow fragment of E. coli DNA Polymerase I. In some embodiments, the DNA polymerase is a truncation of Rev3 as described in Lee et al., PNAS (2014), doi: 10.1073/pnas.1324001111.

In some embodiments, the DNA polymerase comprises any one of SEQ ID NOS: 4-6. In some embodiments, the DNA polymerase has at least 70%, at least 75%, at least 80%, at least 85%, at least 90%, at least 95%, at least 96%, at least 97%, at least 98%, at least 99%, or about 100% sequence identity to any one of SEQ ID NOS: 4-6. In some embodiments, the disclosure provides a polynucleotide which encodes a polypeptide having at least 70%, at least 75%, at least 80%, at least 85%, at least 90%, at least 95%, at least 96%, at least 97%, at least 98%, at least 99%, or about 100% sequence identity to any one of SEQ ID NOS: 4-6. In some embodiments, the DNA polymerase is encoded by a polynucleotide which has been codon optimized for expression in a host cell.

In some embodiments, the fusion protein comprises a DNA ligase. DNA ligase is an enzyme that facilitates the joining of DNA strands together by catalyzing the formation of a phosphodiester bond. DNA ligases can repair single- or double-stranded breaks in DNA. In some embodiments, DNA ligase ligates single-stranded DNA. In some embodiments, DNA ligase ligates blunt ends of double-stranded DNA. In some embodiments, DNA ligase ligates cohesive ends of double-stranded DNA. In some embodiments, the DNA ligase facilitates the recombination of a double-stranded insertion sequence into a double stranded polynucleotide. In some embodiments, when two double-stranded polynucleotide cleavages occur in the target polynucleotide (e.g., at a first target site and a second target site), the DNA ligase can facilitate the recombination of the double-stranded polynucleotide, thereby eliminating the sequence between the first target site and the second target site.

In some embodiments, the Cas nuclease of the fusion protein generates a double-stranded polynucleotide cleavage at a target sequence in a target polynucleotide, e.g., a target DNA sequence. In some embodiments, a template polynucleotide, e.g., a DNA template, is provided, and the DNA ligase of the fusion protein ligates the template polynucleotide to the cleaved target sequence. In some embodiments, the DNA template is a double stranded polynucleotide comprising blunt ends. In some embodiments, the DNA template is a double stranded polynucleotide comprising cohesive ends. In some embodiments, the DNA template is a single stranded polynucleotide.

Exemplary DNA ligases include, but are not limited to, E. coli DNA ligase, Taq DNA ligase, T4 DNA ligase, T7 DNA ligase, DNA ligase I, III, and IV, and Ampligase DNA ligase. In some embodiments, the DNA ligase is T4 ligase.

In some embodiments, the DNA ligase comprises SEQ ID NO: 7. In some embodiments, the DNA ligase has at least 70%, at least 75%, at least 80%, at least 85%, at least 90%, at least 95%, at least 96%, at least 97%, at least 98%, at least 99%, or about 100% sequence identity to SEQ ID NO: 7. In some embodiments, the disclosure provides a polynucleotide which encodes a polypeptide having at least 70%, at least 75%, at least 80%, at least 85%, at least 90%, at least 95%, at least 96%, at least 97%, at least 98%, at least 99%, or about 100% sequence identity to SEQ ID NO: 7. In some embodiments, the DNA ligase is encoded by a polynucleotide which has been codon optimized for expression in a host cell.

In some embodiments, the fusion protein further comprises a DNA-binding or an RNA-binding domain. In some embodiments, the DNA-binding or RNA-binding domain of the fusion protein brings the fusion protein and the template polynucleotide in proximity to one another. In some embodiments, the DNA-binding or RNA-binding domain promotes binding of the template polynucleotide to the fusion protein. In some embodiments, the DNA-binding or RNA-binding domain improves efficiency of the reverse transcriptase, the DNA polymerase, or the DNA ligase reaction by bringing the template polynucleotide and the fusion protein in proximity to one another. In some embodiments, the DNA-binding or RNA-binding domain increases efficiency of incorporating the double-stranded sequence resulting from the reverse transcriptase or DNA polymerase reaction into the cleaved target sequence.

In some embodiments, the fusion protein further comprises a DNA-binding domain. Thus, in some embodiments, the fusion protein comprises a Cas nuclease, a reverse transcriptase, and an DNA-binding domain. In some embodiments, the fusion protein comprises a Cas nuclease, a DNA polymerase, and an DNA-binding domain. In some embodiments, the fusion protein comprises a Cas nuclease, a DNA ligase, and an DNA-binding domain. DNA-binding domains can be found as part of viral, bacterial, and eukaryotic (e.g., mammalian) transcription factors. In some embodiments, the DNA-binding domain binds to single-stranded DNA. In some embodiments, the DNA-binding domain binds to double-stranded DNA. In some embodiments, the DNA-binding protein binds to both single-stranded and double-stranded DNA. Exemplary DNA-binding domains that bind double-stranded DNA include, but are not limited to, helix-turn-helix (HTH), zinc finger (ZF), transcription activation like effector (TALE), small nuclear RNA activating protein (SNAP), leucine zipper, winged helix, helix-loop-helix, HMG-box, Wor3, and OB-fold. Exemplary DNA-binding domains that bind to single-stranded DNA include, but are not limited to, T4 Gene 32 Protein (T4g32), HUH enzymes such as the viral Rep protein, and Far upstream element-binding protein 1 (FUBP). Further DNA-binding domains are provided, e.g., in Alberts B et al. Molecular Biology of the Cell. 4th edition. New York: Garland Science; 2002. DNA-Binding Motifs in Gene Regulatory Proteins; Yesudhas et al., Genes (Basel) 8(8): 192 (2017); and Vidangos et al., Biopolymers 99(12): 1082-1096 (2013). In some embodiments, the DNA-binding domain is a zinc finger DNA-binding domain, a transcription factor, or an adeno-associated virus Rep protein. In some embodiments, the DNA-binding domain is Far upstream element-binding protein (FUBP).

In some embodiments, the fusion protein further comprises an RNA-binding domain. Thus, in some embodiments, the fusion protein comprises a Cas nuclease, a reverse transcriptase, and an RNA-binding domain. In some embodiments, the fusion protein comprises a Cas nuclease, a DNA polymerase, and an RNA-binding domain. In some embodiments, the fusion protein comprises a Cas nuclease, a DNA ligase, and an RNA-binding domain. RNA-binding domains can be found as part of RNA processing proteins, e.g., involved in RNA biogenesis, maturation, transport, cellular localization, and stability. In some embodiments, the RNA-binding domain comprises a RNA-recognition motif In some embodiments, the RNA-binding domain comprises a double-stranded RNA-binding motif. In some embodiments, the RNA-binding domain comprises a zinc finger. In some embodiments, the RNA-binding domain comprises a KH domain such as, e.g., heterogeneous nuclear ribonucleoprotein K (hnRNPK). Exemplary RNA-binding domains include, but are not limited to, NOVA1, ADAR, CPSF, TAP/NXF1:p15, ZBP1, Elav, Sxl, tra-2, FOG-1, MOG-1, MOG-4, MOG-5, RNP-4, GLD-1, GLD-3, DAZ-1, PGL1, OMA-1, OMA2, MEC-8, UNC-75, EXC-7, Pumilio, Nanos, FMRP, CPEB, Staufen 1, FXR1, and MCP2. Further RNA-binding domains are provided, e.g., in Lunde et al., Nat Rev Mol Cell Biol 8(6): 479-490 (2007) and Glisovic et al., FEBS Lett 582(14): 1977-1986 (2008). In some embodiments, the RNA-binding domain is MS2 coat protein (MCP2). In some embodiments, the RNA-binding domain comprises a KH domain. In some embodiments, the RNA-binding domain is hnRNPK.

In some embodiments, the DNA-binding or RNA-binding domain comprises any one of SEQ ID NOS: 8-11. In some embodiments, the DNA-binding or RNA-binding domain comprises a polypeptide sequence having at least 70%, at least 75%, at least 80%, at least 85%, at least 90%, at least 95%, at least 96%, at least 97%, at least 98%, at least 99%, or about 100% sequence identity to any one of SEQ ID NOS: 8-11. In some embodiments, the disclosure provides a polynucleotide which encodes a polypeptide having at least 70%, at least 75%, at least 80%, at least 85%, at least 90%, at least 95%, at least 96%, at least 97%, at least 98%, at least 99%, or about 100% sequence identity to any one of SEQ ID NOS: 8-11.

In some embodiments, the fusion protein provided herein has at least 70%, at least 75%, at least 80%, at least 85%, at least 90%, at least 95%, at least 96%, at least 97%, at least 98%, at least 99%, or about 100% sequence identity to any one of SEQ ID NOS: 18-26.

In some embodiments, the fusion protein further comprises a nuclear localization signal (NLS). As used herein, “nuclear localization signal” or “nuclear localization sequence” (NLS) refers to a polypeptide that “tags” a protein for import into the cell nucleus by nuclear transport, i.e., a protein having a NLS is transported into the cell nucleus. Typically, the NLS includes positively-charged Lys or Arg residues exposed on the protein surface. Exemplary nuclear localization sequences include, but are not limited to, the NLS from: SV40 Large T-Antigen, nucleoplasmin, EGL-13, c-Myc, and TUS-protein. In some embodiments, the NLS includes the sequence PKKKRKV (SEQ ID NO: 14). In some embodiments, the NLS includes the sequence AVKRPAATKKAGQAKKKKLD (SEQ ID NO: 29). In some embodiments, the NLS includes the sequence PAAKRVKLD (SEQ ID NO: 30). In some embodiments, the NLS includes the sequence MSRRRKANPTKLSENAKKLAKEVEN (SEQ ID NO: 31). In some embodiments, the NLS includes the sequence KLKIKRPVK (SEQ ID NO: 32). Other nuclear localization sequences include, but are not limited to, the acidic M9 domain of hnRNP A1, the sequence KIPIK (SEQ ID NO: 33) in yeast transcription repressor Matα2, and PY-NLS.

In some embodiments, the fusion protein further comprises a linker that links the Cas nuclease domain and the reverse transcriptase, DNA polymerase, or DNA ligase. In some embodiments, the linker is of sufficient length and/or flexibility such that the Cas nuclease can be positioned without steric hindrance from the reverse transcriptase, DNA polymerase, or DNA ligase. In some embodiments, the linker is of sufficient length and/or flexibility such that the reverse transcriptase, DNA polymerase, or DNA ligase can perform their respective reactions without steric hindrance from the Cas nuclease. In some embodiments, the linker comprises about 3 to about 100 amino acids in length. In some embodiments, the linker comprises about 5 to about 80 amino acids in length. In some embodiments, the linker comprises about 10 to about 60 amino acids in length. In some embodiments, the linker comprises about 20 to about 50 amino acid sin length. In some embodiments, the linker comprises about 25 to about 40 amino acids in length. Exemplary linker sequences are described herein, e.g., SEQ ID NOS: 15-16.

### Polynucleotides

In some embodiments, the disclosure provides a composition comprising: (a) the fusion protein provided herein; and (b) a polynucleotide that forms a complex with the fusion protein and comprises (i) a guide sequence; and (ii) a template sequence for the reverse transcriptase or the DNA polymerase.

In some embodiments, the polynucleotide of the composition is RNA. In some embodiments, the polynucleotide comprises components of a guide polynucleotide. As described herein, CRISPR/Cas systems include a guide polynucleotide, e.g., a guide RNA. In some embodiments, the guide polynucleotide is RNA. An RNA guide polynucleotide may be referred to herein as “guide RNA,” “gRNA,” or “DNA-targeting RNA.”

In some embodiments, the guide polynucleotide comprises a guide sequence. In some embodiments, the guide polynucleotide comprises a guide sequence and a polypeptide-binding segment. In some embodiments, the guide sequence is capable of hybridizing with a target sequence in a target polynucleotide. In some embodiments, the polypeptide-binding segment of the guide polynucleotide binds to the Cas nuclease. In some embodiments, the polypeptide-binding segment binds to the Cas nuclease of the fusion protein provided herein. In some embodiments, the polypeptide-binding segment binds and/or activates the Cas nuclease.

In some embodiments, the polynucleotide of the composition comprises a guide sequence capable of hybridizing with a target sequence in a target polynucleotide. In some embodiments, the polynucleotide of the composition comprises a polypeptide-binding segment capable of binding to the Cas nuclease of the fusion protein, thereby forming a complex with the fusion protein. In some embodiments, the polynucleotide further comprises a tracrRNA. In some embodiments, the composition further comprises a second polynucleotide comprising a tracrRNA. In some embodiments, the tracrRNA activates the Cas nuclease. In some embodiments, activation of the Cas nuclease initiates or increases its nuclease activity. In some embodiments, activation of the Cas nuclease comprises binding of the nuclease to a target sequence. In some embodiments, the Cas nuclease generates a double-stranded polynucleotide at the target sequence in the target polynucleotide.

In some embodiments, the guide sequence is about 10 to about 40 nucleotides in length. In some embodiments, the guide sequence is about 12 to about 30 nucleotides in length. In some embodiments, the guide sequence is about 15 to about 20 nucleotides in length. In some embodiments, the guide sequence is about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, or about 40 nucleotides in length. In some embodiments, the guide sequence is a sufficient length for hybridizing to the target sequence.

In some embodiments, the polynucleotide of the composition comprises a template sequence. In some embodiments, the template sequence comprises a primer-binding sequence and a sequence of interest. In some embodiments, the template sequence comprises a region of homology to a target sequence. In some embodiments, the region of homology is the primer-binding sequence. In some embodiments, the template sequence comprises a mismatched nucleotide to the target sequence following the primer-binding sequence. In some embodiments, the template sequence comprises 1, 2, 3, 4, 5, 6, 7, 8, 9, or 10 mismatched nucleotides to the target sequence following the primer-binding sequence. As used herein, “mismatched nucleotides” refer to nucleotides that do not form a base pairing. In some embodiments, a template sequence that comprises a mismatched nucleotide has higher insertion frequency as compared to a template sequence that does not comprise a mismatched nucleotide. In some embodiments, the template sequence comprises one or more additional regions of homology to the target sequence. In some embodiments, the template sequence comprises two regions of homology. In some embodiments, the template sequence comprises at least two regions of homology. In some embodiments, the template sequence comprises, in 5′ to 3′ order, a first region of homology, the sequence of interest, and a second region of homology. In some embodiments, the one more additional regions of homology facilitate insertion of the sequence of interest into the target sequence. In some embodiments, the template sequence is single-stranded. In some embodiments, the template sequence is double-stranded. In some embodiments, the template sequence comprises DNA. In some embodiments, the sequence of interest comprises DNA. In some embodiments, the sequence of interest and the primer-binding sequence comprise DNA. In some embodiments, the template sequence comprises RNA. In some embodiments, the template sequence comprises a xeno nucleic acid (XNA). As used herein, XNA refers to a nucleic acid comprising a non-natural backbone in its polymeric chain. For example, in place of the ribose sugar in the DNA or RNA backbone, XNA can include hexose, threose, glycol, cyclohexenyl, desoxyribose, and the like. XNA is further described, e.g., in Schmidt, M. (2010), Bioessays 32(4):322-331. In some embodiments, the template sequence comprises an aptamer. In some embodiments, the template sequence comprises a modification that prevents extension of the sequence of interest by reverse transcriptase and/or DNA polymerase. In some embodiments, the modification comprises an abasic site (also known as an apurinic/apyrimidinic site or AP site), a triethylene glycol (TEG) linker, or both. In some embodiments, the modification prevents overextension of the sequence of interest, thereby increasing the precision of inserting the sequence of interest.

In embodiments where the fusion protein comprises a Cas nuclease and a reverse transcriptase, the polynucleotide comprises a template sequence for the reverse transcriptase. In some embodiments, the Cas nuclease of the fusion protein generates a double-stranded polynucleotide cleavage at a target sequence in a target polynucleotide, e.g., a target DNA sequence, and one strand of the cleaved DNA hybridizes to the primer-binding sequence on the template sequence and serves as a primer for the reverse transcriptase to reverse transcribe the template sequence. In some embodiments, the sequence of interest is reverse transcribed by the reverse transcriptase to generate a first cDNA. In some embodiments, a DNA strand complementary to the first cDNA is generated by a DNA polymerase, thereby generating a double-stranded sequence comprising the sequence of interest. In some embodiments, the double-stranded sequence comprising the sequence of interest is inserted into cleaved target sequence, e.g., via ligation or DNA repair pathways as described herein. In some embodiments, the double-stranded sequence comprising the sequence of interest further comprises a recognition site for an endonuclease, a transposase, or a recombinase, and the endonuclease, transposase, or recombinase integrates the double-stranded sequence into the target polynucleotide. In some embodiments, the regions of homology on the template sequence described herein facilitate insertion of the double-stranded sequence comprising the sequence of interest into cleaved target sequence.

In embodiments where the fusion protein comprises a Cas nuclease and a DNA polymerase, the polynucleotide comprises a template for the DNA polymerase. In some embodiments, the Cas nuclease of the fusion protein generates a double-stranded polynucleotide cleavage at a target sequence in a target polynucleotide, e.g., a target DNA sequence, and one strand of the cleaved DNA hybridizes to the primer-binding sequence on the template sequence and serves as a primer for the DNA polymerase. In some embodiments, the DNA polymerase synthesizes a DNA strand complementary to the sequence of interest, thereby generating a double-stranded sequence comprising the sequence of interest. In some embodiments, the double-stranded sequence comprising the sequence of interest is inserted into cleaved target sequence, e.g., via ligation or DNA repair pathways as described herein. In some embodiments, the double-stranded sequence comprising the sequence of interest further comprises a recognition site for an endonuclease, a transposase, or a recombinase, and the endonuclease, transposase, or recombinase integrates the double-stranded sequence into the target polynucleotide. In some embodiments, the regions of homology on the template sequence described herein facilitate insertion of the double-stranded sequence comprising the sequence of interest into cleaved target sequence.

In some embodiments, the template sequence is about 10 to about 25000 nucleotides in length. In some embodiments, the template sequence is about 15 to about 20000 nucleotides in length. In some embodiments, the template sequence is about 20 to about 15000 nucleotides in length. In some embodiments, the template sequence is about 25 to about 10000 nucleotides in length. In some embodiments, the template sequence is about 10, about 15, about 20, about 25, about 50, about 100, about 150, about 200, about 250, about 300, about 350, about 400, about 450, about 500, about 550, about 600, about 650, about 700, about 750, about 800, about 850, about 900, about 950, about 1000, about 2500, about 5000, about 7500, about 10000, about 15000, about 20000, or about 25000 nucleotides in length. In some embodiments, the template sequence is greater than about 10 nucleotides, greater than about 15 nucleotides, greater than about 20 nucleotides, greater than about 25 nucleotides, greater than about 30 nucleotides, greater than about 35 nucleotides, greater than about 40 nucleotides, greater than about 45 nucleotides, or greater than about 50 nucleotides in length.

In some embodiments, the primer-binding sequence is about 3 to about 50 nucleotides in length. In some embodiments, the primer-binding sequence is about 4 to about 30 nucleotides in length. In some embodiments, the primer-binding sequence is about 5 to about 40 nucleotides in length. In some embodiments, the primer-binding sequence is about 7 to about 30 nucleotides in length. In some embodiments, the primer-binding sequence is about 10 to about 20 nucleotides in length. In some embodiments, the primer-binding sequence is about 3, about 4, about 5, about 6, about 7, about 8, about 9, about 10, about 12, about 15, about 17, about 20, about 22, about 25, about 27, about 30, about 32, about 35, about 38, or about 40 nucleotides in length. In some embodiments, the primer-binding sequence is of sufficient length to hybridize with a region of the cleaved target DNA sequence.

In some embodiments, the sequence of interest is about 1 to about 20000 nucleotides in length. In some embodiments, the sequence of interest is about 2 to about 17000 nucleotides in length. In some embodiments, the sequence of interest is about 3 to about 15000 nucleotides in length. In some embodiments, the sequence of interest is about 4 to about 12000 nucleotides in length. In some embodiments, the sequence of interest is about 5 to about 10000 nucleotides in length. In some embodiments, the sequence of interest is about 10 to about 9000 nucleotides in length. In some embodiments, the sequence of interest is about 50 to about 8000 nucleotides in length. In some embodiments, the sequence of interest is about 100 to about 7000 nucleotides in length. In some embodiments, the sequence of interest is about 200 to about 6000 nucleotides in length. In some embodiments, the sequence of interest is about 500 to about 5000 nucleotides in length. In some embodiments, the sequence of interest is about 5, about 6, about 7, about 8, about 9, about 10, about 15, about 20, about 25, about 30, about 35, about 40, about 45, about 50, about 75, about 100, about 150, about 200, about 250, about 300, about 350, about 400, about 450, about 500, about 550, about 600, about 650, about 700, about 750, about 800, about 850, about 900, about 950, about 1000, about 1250, about 1500, about 1750, about 2000, about 2500, about 3000, about 3500, about 4000, about 4500, about 5000, about 5500, about 6000, about 6500, about 7000, about 7500, about 8000, about 8500, about 9000, about 10000, about 12500, about 15000, about 17500, or about 25000 nucleotides in length. In some embodiments, the sequence of interest is greater than about 5 nucleotides, greater than about 10 nucleotides, greater than about 15 nucleotides, greater than about 20 nucleotides, greater than about 25 nucleotides, greater than about 30 nucleotides, greater than about 35 nucleotides, greater than about 40 nucleotides, greater than about 45 nucleotides, or greater than about 50 nucleotides in length.

In some embodiments, the polynucleotide of the composition further comprises a spacer between the guide sequence and the template sequence. In some embodiments, the spacer comprises a stop sequence for the reverse transcriptase or the DNA polymerase, such that the reverse transcriptase or the DNA polymerase are stopped after transcribing or synthesizing a complementary strand of the sequence of interest. In some embodiments, the spacer comprises more than one stop sequence. In some embodiments, the spacer comprises 1, 2, 3, 4, 5, or more than 5 stop sequences. In some embodiments, multiple stop sequences provide redundancy in stopping the reverse transcriptase or DNA polymerase. In some embodiments, the stop sequence inhibits the activity of the reverse transcriptase and/or DNA polymerase. In some embodiments, the stop sequence promotes dissociation of the reverse transcriptase and/or DNA polymerase from the template sequence.

In some embodiments, the stop sequence comprises a secondary structure. In some embodiments, the secondary structure is an inhibitor of reverse transcriptase and/or DNA polymerase activity. In some embodiments, the secondary structure promotes dissociation of the reverse transcriptase and/or DNA polymerase from the template sequence. In some embodiments, the secondary structure is a hairpin loop (also known as a stem loop). In some embodiments, the secondary structure is a pseudoknot.

In some embodiments, the spacer is about 5 to about 500 nucleotides in length. In some embodiments, the spacer is about 10 to about 400 nucleotides in length. In some embodiments, the spacer is about 10 to about 300 nucleotides in length. In some embodiments, the spacer is about 10 to about 200 nucleotides in length. In some embodiments, the spacer is about 20 to about 150 nucleotides in length. In some embodiments, the spacer is about 30 to about 100 nucleotides in length. In some embodiments, the spacer is about 50 to about 100 nucleotides in length. In some embodiments, the spacer is about 5, about 10, about 15, about 20, about 25, about 30, about 35, about 40, about 45, about 50, about 75, about 100, about 110, about 120, about 130, about 140, about 150, about 160, about 170, about 180, about 190, or about 200 nucleotides in length.

In some embodiments, the disclosure provides a composition comprising: (a) the fusion protein provided herein; (b) a guide polynucleotide that forms a complex with the fusion protein and comprises a guide sequence; and (c) a template polynucleotide comprising a template sequence for the reverse transcriptase or the DNA polymerase.

Guide polynucleotides are described herein. In some embodiments, the guide polynucleotide of the composition comprises a guide sequence capable of hybridizing with a target sequence. In some embodiments, the guide polynucleotide of the composition comprises a polypeptide-binding segment capable of binding to the Cas nuclease of the fusion protein, thereby forming a complex with the fusion protein. In some embodiments, the guide polynucleotide further comprises a tracrRNA. In some embodiments, the composition further comprises a third polynucleotide comprising a tracrRNA. In some embodiments, the tracrRNA activates the Cas nuclease. In some embodiments, activation of the Cas nuclease initiates or increases its nuclease activity. In some embodiments, activation of the Cas nuclease comprises binding of the nuclease to a target sequence.

In some embodiments, the guide sequence is about 10 to about 40 nucleotides in length. In some embodiments, the guide sequence is about 12 to about 30 nucleotides in length. In some embodiments, the guide sequence is about 15 to about 20 nucleotides in length. In some embodiments, the guide sequence is about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, or about 40 nucleotides in length. In some embodiments, the guide sequence is a sufficient length for hybridizing to a target sequence.

Components of the template polynucleotide, e.g., the template sequence for the reverse transcriptase or the DNA polymerase, primer-binding sequence, stop sequence, sequence of interest, and/or additional regions of homology, are described herein. In some embodiments, the template sequence is about 10 to about 25000 nucleotides in length. In some embodiments, the template sequence is about 15 to about 20000 nucleotides in length. In some embodiments, the template sequence is about 20 to about 15000 nucleotides in length. In some embodiments, the template sequence is about 25 to about 10000 nucleotides in length. In some embodiments, the template sequence is about 10, about 15, about 20, about 25, about 50, about 100, about 150, about 200, about 250, about 300, about 350, about 400, about 450, about 500, about 550, about 600, about 650, about 700, about 750, about 800, about 850, about 900, about 950, about 1000, about 2500, about 5000, about 7500, about 10000, about 15000, about 20000, or about 25000 nucleotides in length. In some embodiments, the template sequence is greater than about 10 nucleotides, greater than about 15 nucleotides, greater than about 20 nucleotides, greater than about 25 nucleotides, greater than about 30 nucleotides, greater than about 35 nucleotides, greater than about 40 nucleotides, greater than about 45 nucleotides, or greater than about 50 nucleotides in length.

In some embodiments, the template sequence comprises a sequence of interest. In some embodiments, the sequence of interest is about 1 to about 20000 nucleotides in length. In some embodiments, the sequence of interest is about 2 to about 17000 nucleotides in length. In some embodiments, the sequence of interest is about 3 to about 15000 nucleotides in length. In some embodiments, the sequence of interest is about 4 to about 12000 nucleotides in length. In some embodiments, the sequence of interest is about 5 to about 10000 nucleotides in length. In some embodiments, the sequence of interest is about 10 to about 9000 nucleotides in length. In some embodiments, the sequence of interest is about 50 to about 8000 nucleotides in length. In some embodiments, the sequence of interest is about 100 to about 7000 nucleotides in length. In some embodiments, the sequence of interest is about 200 to about 6000 nucleotides in length. In some embodiments, the sequence of interest is about 500 to about 5000 nucleotides in length. In some embodiments, the sequence of interest is about 5, about 6, about 7, about 8, about 9, about 10, about 15, about 20, about 25, about 30, about 35, about 40, about 45, about 50, about 75, about 100, about 150, about 200, about 250, about 300, about 350, about 400, about 450, about 500, about 550, about 600, about 650, about 700, about 750, about 800, about 850, about 900, about 950, about 1000, about 1250, about 1500, about 1750, about 2000, about 2500, about 3000, about 3500, about 4000, about 4500, about 5000, about 5500, about 6000, about 6500, about 7000, about 7500, about 8000, about 8500, about 9000, about 10000, about 12500, about 15000, about 17500, or about 25000 nucleotides in length. In some embodiments, the sequence of interest is greater than about 5 nucleotides, greater than about 10 nucleotides, greater than about 15 nucleotides, greater than about 20 nucleotides, greater than about 25 nucleotides, greater than about 30 nucleotides, greater than about 35 nucleotides, greater than about 40 nucleotides, greater than about 45 nucleotides, or greater than about 50 nucleotides in length.

In some embodiments, the template polynucleotide further comprises a primer-binding sequence as described herein. In some embodiments, the primer-binding sequence is about 3 to about 50 nucleotides in length. In some embodiments, the primer-binding sequence is about 4 to about 30 nucleotides in length. In some embodiments, the primer-binding sequence is about 5 to about 40 nucleotides in length. In some embodiments, the primer-binding sequence is about 7 to about 30 nucleotides in length. In some embodiments, the primer-binding sequence is about 10 to about 20 nucleotides in length. In some embodiments, the primer-binding sequence is about 3, about 4, about 5, about 6, about 7, about 8, about 9, about 10, about 12, about 15, about 17, about 20, about 22, about 25, about 27, about 30, about 32, about 35, about 38, or about 40 nucleotides in length. In some embodiments, the guide sequence is a sufficient length for hybridizing to a target sequence that has been cleaved by the Cas nuclease of the fusion protein.

In some embodiments, the template polynucleotide further comprises a stop sequence for the reverse transcriptase or the DNA polymerase as described herein. In some embodiments, the template polynucleotide comprises more than one stop sequence. In some embodiments, the spacer comprises 1, 2, 3, 4, 5, or more than 5 stop sequences. In some embodiments, the stop sequence comprises a secondary structure. In some embodiments, the secondary structure is an inhibitor of reverse transcriptase and/or DNA polymerase activity. In some embodiments, the secondary structure promotes dissociation of the reverse transcriptase and/or DNA polymerase from the template sequence. In some embodiments, the secondary structure is a hairpin loop (also known as a stem loop). In some embodiments, the secondary structure is a pseudoknot.

In embodiments where the fusion protein further comprises a DNA-binding or RNA-binding domain, the template polynucleotide further comprises a sequence capable of binding to the DNA-binding or RNA-binding domain. Non-limiting examples of DNA sequences for binding to DNA-binding domains such as, e.g., zinc finger DNA-binding domain, transcription factor, adeno-associated viral Rep protein, for FUBP, are described in, e.g., Bulyk et al., Proc Natl Acad Sci USA 98(13): 7158-7163 (2001); Fornes et al., Nucleic Acids Res 2019; doi:10.1093/nar/gkz1001; Gearing et al., PLOS One 14(9): e0215495 (2019); Wonderling et al., J Virol 71(3): 2528-2534 (1997); Benjamin et al., Proc Natl Acad Sci USA 105(47): 18296-18301 (2008), and Hudson et al., Nat Rev Mol Cell Biol 15(11): 749-760 (2014). Non-limiting examples of RNA sequences for binding to RNA-binding domains such as, e.g., MCP2, are described in, e.g., Castello et al., Mol Cell 63: 696-710 (2016); Rube et al., Nat Comm 7: 11025 (2016); Peabody et al., EMBO J 12(2): 595-600 (1993), and Hudson et al., Nat Rev Mol Cell Biol 15(11): 749-760 (2014).

In some embodiments, the template polynucleotide comprises an adeno-associated virus (AAV) vector comprising a sequence of interest. AAV is a non-enveloped virus that can be engineered to deliver sequences of interest into target cells. See, e.g., Naso et al., BioDrugs 31(4): 317-334 (2017). In some embodiments, the AAV vector is single-stranded DNA. In some embodiments, the AAV vector comprises an inverted terminal repeat (ITR), a promoter, the sequence of interest, and a terminator. In some embodiments, the AAV vector comprises an ITR and the sequence of interest. In some embodiments, the AAV vector does not comprise a viral gene. In some embodiments, the template polynucleotide comprises an AAV vector, and the fusion protein comprises a Cas nuclease and a DNA polymerase. In some embodiments, the AAV vector is about 200, about 300, about 400, about 500, about 600, about 700, about 800, about 900, about 1000, about 2000, about 3000, about 4000, or about 5000 nucleotides in length. In some embodiments, the sequence of interest in the AAV vector is about 10, about 20, about 30, about 40, about 50, about 60, about 70, about 80, about 90, about 100, about 200, about 300, about 400, about 500, about 600, about 700, about 800, about 900, about 1000, about 1200, about 1500, about 1700, about 2000, about 2200, about 2500, about 2700, about 3000, about 3200, about 3500, about 3700, about 4000, about 4200, about 4500, or about 4700 nucleotides in length.

In some embodiments, the disclosure provides a polynucleotide encoding the fusion protein provided herein. In some embodiments, the polynucleotide encodes a polypeptide having having at least 70%, at least 75%, at least 80%, at least 85%, at least 90%, at least 95%, at least 96%, at least 97%, at least 98%, at least 99%, or about 100% sequence identity to any one of SEQ ID NOS: 18-26.

In some embodiments, the polynucleotides herein, e.g., the polynucleotide encoding the fusion protein, the polynucleotide comprising the guide sequence and the template sequence, the guide polynucleotide, and/or the template polynucleotide, are codon optimized for expression in a eukaryotic cell. In some embodiments, the polynucleotides herein are codon optimized for expression in a bacterial cell. In some embodiments, the polynucleotides herein are codon optimized for expression in a mammalian cell. In some embodiments, the polynucleotides herein are codon optimized for expression in a human cell. As used herein, “codon optimization” refers to the adjustment of codons to match the expression host's tRNA abundance in order to increase yield and efficiency of recombinant or heterologous protein expression. Codon optimization methods are known in the art and may be performed using software programs such as, for example, the Codon Optimization tool from Integrated DNA Technologies, the Codon Usage Table analysis tool from Entelechon, the Blue Heron software from GENEMAKER, the Gene Forge software from Aptagen, and other software such as DNA Builder, OPTIMIZER, and the OptimumGene algorithm.

In some embodiments, the disclosure provides a vector comprising the polynucleotide encoding the fusion protein provided herein. In some embodiments, the disclosure provides a vector comprising: the polynucleotide encoding the fusion protein, the polynucleotide comprising the guide sequence and the template sequence, the guide polynucleotide, the template polynucleotide, or a combination thereof. In some embodiments, the polynucleotide encoding the fusion protein and the polynucleotide comprising the guide sequence and the template sequence are on a single vector. In some embodiments, the polynucleotide encoding the fusion protein and the polynucleotide comprising the guide sequence and the template sequence are on one or more vectors. In some embodiments, the polynucleotide encoding the fusion protein, the guide polynucleotide, and the template oligonucleotide are on a single vector. In some embodiments, the polynucleotide encoding the fusion protein, the guide polynucleotide, and the template oligonucleotide are on one or more vectors.

Various types of vectors, e.g., viral and non-viral vectors, are provided herein. In some embodiments, the vector is an expression vector. In some embodiments, the vector is a bacterial expression vector. In some embodiments, the vector is a mammalian expression vector. In some embodiments, the vector is a human expression vector. In some embodiments, the vector is a plant expression vector.

In some embodiments, the vector is a viral vector. In some embodiments, the viral vector is a retrovirus, adeno-associated virus, pox, baculovirus, vaccinia, herpes simplex, Epstein-Barr virus, adenovirus, geminivirus, or caulimovirus vector. In some embodiments, the viral vector is an adenovirus, a lentivirus, or an adeno-associated viral vector. Viral transduction with adenovirus, adeno-associated virus (AAV), and lentiviral vectors (wherein administration can be local, targeted or systemic) have been used as delivery methods for in vivo gene therapy. Methods of introducing vectors, e.g., viral vectors, into cells (e.g., transfection) are described herein.

In some embodiments, the vector further comprises a regulatory element operably linked to the polynucleotide encoding the fusion protein, the polynucleotide comprising the guide sequence and the template sequence, the guide polynucleotide, and/or the template polynucleotide. In some embodiments, the regulatory element is a bacterial promoter. In some embodiments, the regulatory element is a viral promoter. In some embodiments, the regulatory element is a mammalian promoter. In some embodiments, the regulatory element is a terminator. Regulatory elements are further described herein.

In some embodiments, the fusion protein, the polynucleotide comprising the guide sequence and the template sequence, the guide polynucleotide, and/or the template polynucleotide are introduced into a cell via a delivery particle. Delivery particles can be used to deliver exogenous biological materials such as, e.g., polynucleotides and proteins described herein. In some embodiments, the delivery particle is a solid, a semi-solid, an emulsion, or a colloid. In some embodiments, the delivery particle is a lipid-based particle, a liposome, a micelle, a vesicle, or an exosome. In some embodiments, the delivery particle is a nanoparticle. Delivery particles are further described, e.g., in US 2011/0293703, US 2012/0251560, US 2013/0302401, U.S. Pat. No. 5,543,158, 5,855,913, 5,895,309, 6,007,845, and 8,709,843.

In some embodiments, the fusion protein, the polynucleotide comprising the guide sequence and the template sequence, the guide polynucleotide, and/or the template polynucleotide are introduced into a cell via a vesicle. In some embodiments, the vesicle comprises an exosome or a liposome. Engineered vesicles for delivery of exogenous biological materials into target cells are described, e.g., in Alvarez-Erviti et al., Nat Biotechnol 29:341 (2011), El-Andaloussi et al., Nat Protocols 7:2112-2116 (2012), Wahlgren et al., Nucleic Acid Res 40(17):e130 (2012), Morrissey et al., Nat Biotechnol 23(8):1002-1007 (2005), Zimmerman et al., Nat Letters 441:111-114 (2006), and Li et al., Gene Therapy 19:775-780 (2012).

### Cells

In some embodiments, the disclosure provides a cell comprising the fusion protein provided herein. In some embodiments, the disclosure provides a cell comprising the polynucleotide encoding the fusion protein provided herein. In some embodiments, the disclosure provides a cell comprising the polynucleotide encoding the fusion protein, the polynucleotide comprising the guide sequence and the template sequence, the guide polynucleotide, the template polynucleotide, or a combination thereof. In some embodiments, the disclosure provides a cell comprising the vector provided herein, e.g., comprising the polynucleotide encoding the fusion protein, the polynucleotide comprising the guide sequence and the template sequence, the guide polynucleotide, the template polynucleotide, or a combination thereof

In some embodiments, the cell is a bacterial cell. In some embodiments, the bacterial cell is a laboratory strain. Examples of such bacterial cells include, but are not limited to, E. coli, S. aureus, V. cholerae, S. pneumoniae, B. subtilis, C. crescentus, M genitalium, A. fischeri, Synechocystis, P. fluorescens, A. vinelandii, S. coelicolor. In some embodiments, the bacterial cell is of bacteria used in preparation of food and/or beverages. Non-limiting exemplary genera of such cells include, but are not limited to, Acetobacter, Arthrobacter, Bacillus, Bifidobacterium, Brachybacterium, Brevibacterium, Carnobacterium, Corynebacterium, Enterococcus, Gluconacetobacter, Hafnia, Halomonas, Kocuria, Lactobacillus (including L. acetotolerans, L. acidipiscis, L. acidophilus, L. alimentarius, L. brevis, L. bucheri, L. casei, L. curvatus, L. fermentum, L. hilgardii, L. jensenii, L. kimchii, L. lactis, L. paracasei, L. plantarum, and L. sakei), Leuconostoc, Microbacterium, Pediococcus, Propionibacterium, Weissella, and Zymomonas.

In some embodiments, the cell is a eukaryotic cell. In some embodiments, the eukaryotic cell is a mammalian cell. In some embodiments, the eukaryotic cell is an animal cell. In some embodiments, the eukaryotic cell is a mammalian cell. In some embodiments, the eukaryotic cell is of an animal or human cell, cell line, or cell strain. Examples of animal or mammalian cells, cell lines, or cell strains include, but are not limited to, mouse myeloma (NSO), Chinese hamster ovary (CHO), HT1080, H9, HepG2, MCF7, MDBK Jurkat, NIH3T3, PC12, BHK (baby hamster kidney), EBX, EB14, EB24, EB26, EB66, or Ebv13, VERO, SP2/0, YB2/0, Y0, C127, L cell, COS (e.g., COS1 and COS7), QC1-3, HEK293, VERO, PER.C6, HeLA, EB1, EB2, EB3, oncolytic cell, or hybridoma cell. In some embodiments, the eukaryotic cell is a CHO cell. In some embodiments, the cell is a CHO-K1 cell, a CHO-K1 SV cell, a DG44 CHO cell, a DUXB11 CHO cell, a CHOS, a CHO GS knock-out cell, a CHO FUT8 GS knock-out cell, a CHOZN, or a CHO-derived cell. The CHO GS knock-out cell (e.g., GSKO cell) can be, for example, a CHO-K1 SV GS knockout cell.

In some embodiments, the eukaryotic cell is a human stem cell. The stem cells can be, for example, pluripotent stem cells, including embryonic stem cells (ESCs), adult stem cells, induced pluripotent stem cells (iPSCs), tissue specific stem cells (e.g., hematopoietic stem cells) and mesenchymal stem cells (MSCs). In some embodiments, the cell is a differentiated form of any of the cells described herein. In some embodiments, the eukaryotic cell is a cell derived from any primary cell in culture.

In some embodiments, the eukaryotic cell is a hepatocyte such as a human hepatocyte, animal hepatocyte, or a non-parenchymal cell. For example, the eukaryotic cell can be a plateable metabolism qualified human hepatocyte, a plateable induction qualified human hepatocyte, plateable human hepatocyte, suspension qualified human hepatocyte (including 10-donor and 20-donor pooled hepatocytes), human hepatic kupffer cells, human hepatic stellate cells, dog hepatocytes (including single and pooled Beagle hepatocytes), mouse hepatocytes (including CD-1 and C57BI/6 hepatocytes), rat hepatocytes (including Sprague-Dawley, Wistar Han, and Wistar hepatocytes), monkey hepatocytes (including Cynomolgus or Rhesus monkey hepatocytes), cat hepatocytes (including Domestic Shorthair hepatocytes), and rabbit hepatocytes (including New Zealand White hepatocytes).

In some embodiments, the eukaryotic cell is a plant cell. For example, the plant cell can be of a crop plant such as cassava, corn, sorghum, wheat, or rice. The plant cell can be of an algae, tree, or vegetable. The plant cell can be of a monocot or dicot or of a crop or grain plant, a production plant, fruit, or vegetable. For example, the plant cell can be of a tree, e.g., a citrus tree such as orange, grapefruit, or lemon tree; peach or nectarine trees; apple or pear trees; nut trees such as almond or walnut or pistachio trees; nightshade plants, e.g., potato, tomato, eggplant, pepper, paprika; plants of the genus Brassica, plants of the genus Lactuca; plants of the genus Spinacia; plants of the genus Capsicum; cotton, tobacco, asparagus, carrot, cabbage, broccoli, cauliflower, lettuce, spinach, strawberry, blueberry, raspberry, blackberry, grape, coffee, cocoa, and the like.

### Methods of Site-Specific Modification

In some embodiments, the disclosure provides a method of providing a site-specific modification at a target sequence in a target polynucleotide, the method comprising contacting the target polynucleotide with the composition provided herein. In some embodiments, the composition comprises (a) the fusion protein described herein and (b) the polynucleotide described herein comprising the guide sequence and the template sequence. In some embodiments, the composition comprises (a) the fusion protein described herein, the (b) the guide polynucleotide described herein, and (c) the template oligonucleotide described herein. In some embodiments, the target polynucleotide is double-stranded. In some embodiments, the target polynucleotide is DNA.

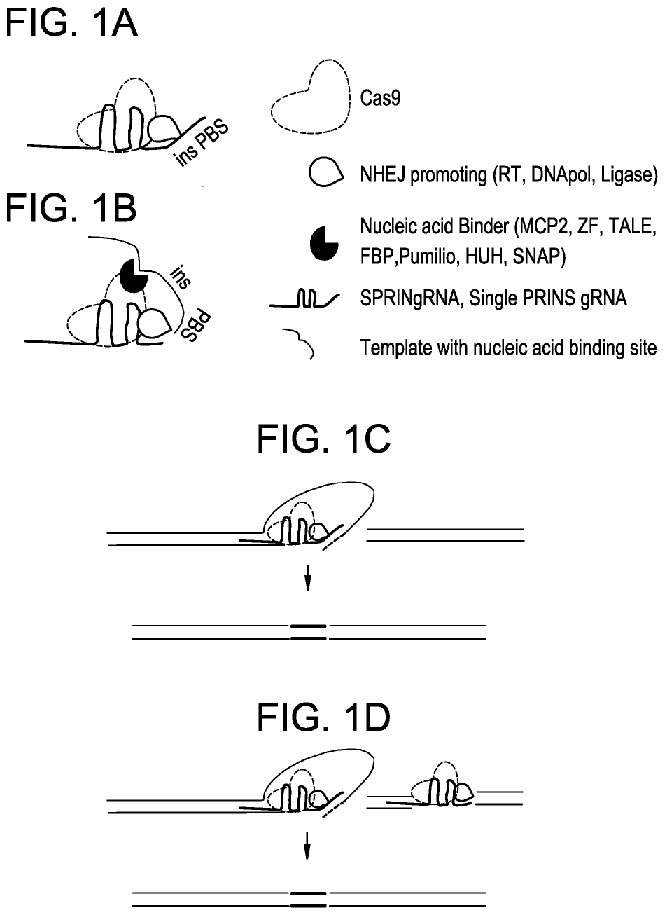

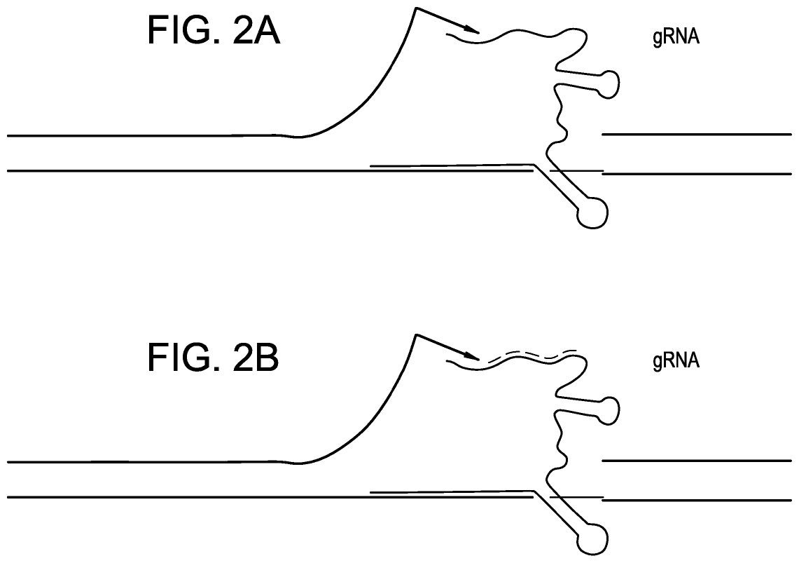

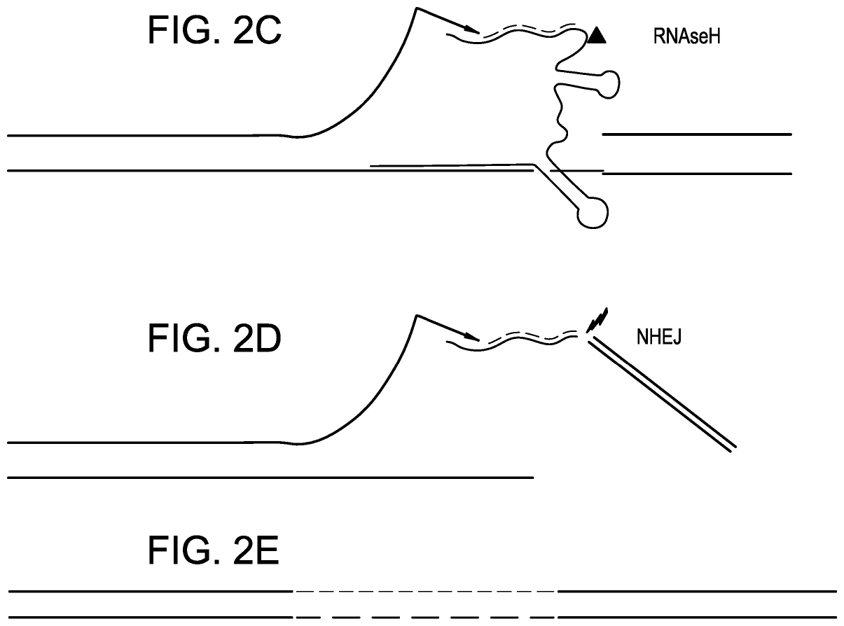

An exemplary method is illustrated in FIGS. 1 and 2. FIGS. 1A and 1B show a Cas9 fused to an “NHEJ-promoting domain,” e.g., a reverse transcriptase, DNA polymerase, or DNA ligase. In FIG. 1A, the “SPRINgRNA” (single primed insertion guide RNA) comprises an sequence of interest (“ins”) and a primer-binding site (PBS). In FIG. 1B, the fusion protein further comprises a DNA- or RNA-binding domain (e.g., MCP2, ZF, TALE, FBP, Pumilio, HUH, or SNAP), and the sequence of interest with the PBS is provided as separate polynucleotide. FIG. 1C shows the mechanism of action of the PRINS complex depicted in FIG. 1A. The Cas9 nuclease generates a double-stranded cleavage at the target polynucleotide. The template sequence in the Cas9 complex containing the PBS and sequence of interest is used to copy the sequence of interest. The double stranded sequence generated can then be ligated by NHEJ to the cleaved target polynucleotide.

In some embodiments, the fusion protein comprises a Cas nuclease and a reverse transcriptase. In some embodiments, the template sequence comprises RNA. In some embodiments, the guide sequence of the polynucleotide or the guide polynucleotide in the composition is capable of hybridizing to the target sequence. In some embodiments, the fusion protein is guided to the target sequence via hybridization of the guide sequence and the target sequence. In some embodiments, the contacting step of the method is performed under conditions sufficient for the Cas nuclease to generate a double-stranded polynucleotide cleavage at the target sequence. In some embodiments, one strand of the cleaved target sequence is a primer for the reverse transcriptase. In some embodiments, the template sequence of the polynucleotide or the template polynucleotide in the composition comprises a primer-binding site capable of binding to the primer. In some embodiments, the template sequence comprises a sequence of interest. In some embodiments, the contacting step of the method is performed under conditions sufficient for the reverse transcriptase to recognize the primer-binding sequence hybridized to the target sequence and reverse transcribe a complementary strand of the sequence of interest to generate a first cDNA. In some embodiments, a DNA polymerase synthesizes a DNA strand complementary to the first cDNA. In some embodiments, the template sequence is removed from the first cDNA by an RNase so that the DNA polymerase can synthesize a DNA strand complementary to the first cDNA, thereby producing a double stranded sequence comprising the sequence of interest. In some embodiments where the reverse transcriptase is capable of RNase activity, the template sequence is removed by the reverse transcriptase. In some embodiments, the method further comprises providing an RNase to remove the template sequence. In some embodiments, the RNase is RNase H. RNase H is capable of specifically hydrolyzing RNA that is hybridized to DNA.

In some embodiments, after removal, e.g., digestion or cleavage, of the template sequence from the first cDNA by the RNase, e.g., RNase H, a DNA polymerase generates a DNA strand complementary to the first cDNA, thereby producing a double stranded sequence comprising the sequence of interest. In some embodiments where the reverse transcriptase is capable of DNA polymerase activity, the DNA strand complementary to the first cDNA is generated by the reverse transcriptase. In some embodiments where the method is performed in a cell, the DNA strand complementary to the first cDNA is generated by a native DNA polymerase in the cell. In some embodiments where the method is performed in vitro, the method further comprises providing a DNA polymerase to generate the DNA strand complementary to the first cDNA. In some embodiments, the first cDNA and the DNA strand complementary to the first cDNA hybridize to form a double-stranded sequence comprising the sequence of interest. In some embodiments, the double-stranded sequence comprising the sequence of interest is capable of being inserted into the cleaved target sequence. In some embodiments, the double-stranded sequence comprising the sequence of interest is inserted into the cleaved target sequence by a DNA repair pathway, e.g., non-homologous end joining (NHEJ). In some embodiments, the double-stranded sequence comprising the sequence of interest is inserted into the cleaved target sequence by a DNA ligase. In some embodiments, the double-stranded sequence comprising the sequence of interest further comprises a recognition site for an endonuclease, a transposase, or a recombinase, and the endonuclease, transposase, or recombinase integrates the double-stranded sequence into the target polynucleotide. In some embodiments, the regions of homology on the template sequence described herein facilitate insertion of the double-stranded sequence comprising the sequence of interest into cleaved target sequence.

In some embodiments, the fusion protein comprises a Cas nuclease and a DNA polymerase. In some embodiments, the template sequence comprises DNA. In some embodiments, the template sequence comprises single-stranded DNA (ssDNA). In some embodiments, the guide sequence of the polynucleotide or the guide polynucleotide in the composition is capable of hybridizing to the target sequence. In some embodiments, the fusion protein is guided to the target sequence via hybridization of the guide sequence and the target sequence. In some embodiments, the contacting step of the method is performed under conditions sufficient for the Cas nuclease to generate a double-stranded polynucleotide cleavage at the target sequence. In some embodiments, one strand of the cleaved target sequence is a primer for the DNA polymerase. In some embodiments, the template sequence of the polynucleotide or the template polynucleotide in the composition comprises a primer-binding site capable of binding to the primer. In some embodiments, the template sequence comprises a sequence of interest. In some embodiments, the contacting step of the method is performed under conditions sufficient for the DNA polymerase to recognize the primer-binding sequence hybridized to the target sequence and generate a double-stranded sequence comprising the sequence of interest. In some embodiments, the double-stranded sequence comprising the sequence of interest is capable of being inserted into the cleaved target sequence. In some embodiments, the double-stranded sequence comprising the sequence of interest is inserted into the cleaved target sequence by a DNA repair pathway, e.g., non-homologous end joining (NHEJ). In some embodiments, the double-stranded sequence comprising the sequence of interest is inserted into the cleaved target sequence by a DNA ligase. In some embodiments, the double-stranded sequence comprising the sequence of interest further comprises a recognition site for an endonuclease, a transposase, or a recombinase, and the endonuclease, transposase, or recombinase integrates the double-stranded sequence into the target polynucleotide. In some embodiments, the regions of homology on the template sequence described herein facilitate insertion of the double-stranded sequence comprising the sequence of interest into cleaved target sequence.

In some embodiments, the method further comprises generating a second double-stranded polynucleotide cleavage at a second target sequence in the target polynucleotide. In some embodiments, the second target sequence is upstream of the target sequence. In some embodiments, the second target sequence is downstream of the target sequence. In some embodiments, the second double-stranded polynucleotide cleavage is generated by a second Cas nuclease. In some embodiments, one end of the double-stranded sequence comprising the sequence of interest, e.g., generated by the reverse transcriptase and/or the DNA polymerase, is joined with the cleaved target sequence, and the other end of the double-stranded sequence is joined with the cleaved second target sequence, thereby replacing the sequence of the target polynucleotide between the target sequence and the second target sequence. Such an embodiment is exemplified in FIG. 1D. The Cas9 nuclease generates a double-stranded break at the target polynucleotide. The template sequence in the Cas9 complex containing the PBS and sequence of interest is used to copy the sequence of interest. The double stranded sequence generated can then be ligated by NHEJ to another break generated downstream by a second CRISPR/Cas complex. The sequence on the target polynucleotide between the two CRISPR/Cas complexes is replaced by the sequence of interest.

In some embodiments, the double-stranded sequence comprising the sequence of interest is inserted into the cleaved target sequence by a DNA repair pathway. In embodiments where the method is performed in a cell, the double-stranded sequence is inserted into the target sequence by DNA repair pathway components native to the cell. DNA repair pathways include the non-homologous end joining (NHEJ) pathway, microhomology-mediated end joining (MMEJ) pathway, and the homology-directed repair (HDR) pathway. NHEJ does not require a homologous template. In general, NHEJ has higher repair efficiency but lower fidelity when compared with HDR, although errors decrease when the double-stranded breaks have compatible cohesive ends or overhangs. MMEJ, which has micro-homologies (e.g., of about 2 to about 10 base pairs) on both sides of a double-stranded break. HDR requires a homologous template to direct repair, and HDR repairs are typically high-fidelity but low efficiency compared with NHEJ and MMEJ. In some embodiments, the method is performed under conditions sufficient for non-homologous end joining (NHEJ).

In some embodiments, the double-stranded sequence comprising the sequence of interest, e.g., generated by the reverse transcriptase and/or the DNA polymerase, is inserted into the cleaved target sequence by ligation. In some embodiments, the ligation is performed by a ligase, e.g., a DNA ligase. In some embodiments, the method further comprises providing a ligase. Ligases are further described herein. In some embodiments, the ligase is T4 DNA ligase.

In some embodiments, the double-stranded sequence comprising the sequence of interest, e.g., generated by the reverse transcriptase and/or the DNA polymerase, further comprises a recognition site for an endonuclease, a transposase, or a recombinase. In some embodiments, the endonuclease, transposase, or recombinase integrates the double-stranded sequence into the target polynucleotide. Mechanisms of sequence integration by endonucleases, transposases, and recombinases are known to one of skill in the art and are further described, e.g., in Carlson et al., Mol Microbiol 27(4): 671-676 (1998), Nesmelova et al., Adv Drug Deliv Rev 62: 1187-1195 (2010), and Hallet et al., FEMS Microbiol Rev 21(2): 157-178 (1997).

In some embodiments, the fusion protein comprises Cas nuclease and a DNA ligase, and the composition comprises a double-stranded template polynucleotide, wherein the double-stranded template polynucleotide comprises a sequence of interest. In some embodiments, the guide sequence of the polynucleotide or the guide polynucleotide in the composition is capable of hybridizing to the target sequence. In some embodiments, the fusion protein is guided to the target sequence via hybridization of the guide sequence and the target sequence. In some embodiments, the contacting step of the method is performed under conditions sufficient for the Cas nuclease to generate a double-stranded polynucleotide cleavage at the target sequence. In some embodiments, the double-stranded template polynucleotide is capable of being inserted into the cleaved target sequence by ligation. In some embodiments, the template sequence and the cleaved target sequence comprise complementary cohesive ends, and the DNA ligase is capable of ligating cohesive ends. In some embodiments, the template sequence and the cleave target sequence comprise blunt ends, and the DNA ligase is capable of ligating blunt ends. In some embodiments, the contacting step of the method is performed under conditions sufficient for the DNA ligase to ligate the template sequence comprising the sequence of interest to the cleaved target sequence, thereby incorporating the template sequence into the cleaved target sequence. Ligases are further described herein. In some embodiments, the ligase is T4 DNA ligase. In some embodiments, the fusion protein comprises Cas nuclease and a DNA ligase, and the template sequence comprises a sequence of interest and a primer-binding sequence, and the method further comprises contacting the target polynucleotide with a reverse transcriptase. In some embodiments, the reverse transcriptase reverse transcribes a complementary strand of the sequence of interest, thereby forming a double-stranded sequence comprising the sequence of interest as described herein. In some embodiments, the DNA ligase of the fusion protein ligates the double-stranded sequence into the cleaved target sequence.

In some embodiments where the composition comprises the polynucleotide comprising a guide sequence and a template sequence, the template sequence is in proximity to the cleavage site and to the fusion protein. In some embodiments where the composition comprises the template polynucleotide, the fusion protein further comprises a DNA-binding domain or an RNA-binding domain to bind the template polynucleotide, thereby bringing the template sequence in proximity to the cleavage site and to the fusion protein. In some embodiments, proximity of the template sequence to the fusion protein promotes activity of the reverse transcriptase, DNA polymerase, or DNA ligase. In some embodiments, proximity of the template sequence to the cleavage site promotes incorporation of the double-stranded sequence resulting from the reverse transcriptase or DNA polymerase reaction into the cleaved target sequence.

In some embodiments, the present method increases efficiency of incorporating the double-stranded sequence into the cleaved target sequence by providing the double-stranded sequence in proximity to the cleaved target sequence. In some embodiments, the present method increases efficiency of incorporating the double-stranded sequence into the cleaved target sequence by reducing re-ligation of the cleaved target sequence. In some embodiments, the present method has improved efficiency compared with a method that utilizes a Cas nuclease without a fused reverse transcriptase, DNA polymerase, or DNA ligase to generate a double-stranded cleavage. In some embodiments, the present method has at least 2-fold, at least 5-fold, at least 10-fold, at least 20-fold, at least 30-fold, at least 40-fold, at least 50-fold, at least 60-fold, at least 70-fold, at least 80-fold, at least 90-fold, at least 100-fold, least 150-fold, or at least 200-fold or higher efficiency compared with a method that utilizes a Cas nuclease without a fused reverse transcriptase, DNA polymerase, or DNA ligase to generate a double-stranded cleavage. In some embodiments, the present method has improved efficiency compared with a method that that does not bring a sequence of interest in proximity to the cleaved target sequence. In some embodiments, the present method has at least 2-fold, at least 5-fold, at least 10-fold, at least 20-fold, at least 30-fold, at least 40-fold, at least 50-fold, at least 60-fold, at least 70-fold, at least 80-fold, at least 90-fold, at least 100-fold, least 150-fold, or at least 200-fold or higher efficiency compared with a method that that does not bring a sequence of interest in proximity to the cleaved target sequence.

In some embodiments, the present method is capable of inserting a long sequence of interest into a target sequence. For example, the present method is capable of inserting a sequence of about 10,000 nucleotides in length into a target sequence, so long as the reverse transcriptase or DNA polymerase has the processivity to generate a sequence of such length. Examples of reverse transcriptase and DNA polymerase with high processivity are provided herein. In some embodiments, the sequence of interest is greater than about 5 nucleotides, greater than about 10 nucleotides, greater than about 15 nucleotides, greater than about 20 nucleotides, greater than about 25 nucleotides, greater than about 30 nucleotides, greater than about 35 nucleotides, greater than about 40 nucleotides, greater than about 45 nucleotides, or greater than about 50 nucleotides in length. In some embodiments, the sequence of interest is about 1 to about 20000 nucleotides in length. In some embodiments, the sequence of interest is about 2 to about 17000 nucleotides in length. In some embodiments, the sequence of interest is about 3 to about 15000 nucleotides in length. In some embodiments, the sequence of interest is about 4 to about 12000 nucleotides in length. In some embodiments, the sequence of interest is about 5 to about 10000 nucleotides in length. In some embodiments, the sequence of interest is about 10 to about 9000 nucleotides in length. In some embodiments, the sequence of interest is about 50 to about 8000 nucleotides in length. In some embodiments, the sequence of interest is about 100 to about 7000 nucleotides in length. In some embodiments, the sequence of interest is about 200 to about 6000 nucleotides in length. In some embodiments, the sequence of interest is about 500 to about 5000 nucleotides in length.

In some embodiments, the method is performed in vitro. In some embodiments, the method is performed in a cell. Examples of cells are provided herein.

### Kits

In some embodiments, the disclosure provides a kit comprising the fusion protein provided herein. In some embodiments, the fusion protein in the kit is provided as a polynucleotide encoding the fusion protein. In some embodiments, the polynucleotide encoding the fusion protein is provided on a vector, e.g., a vector described herein.

In some embodiments, the kit further comprises a polynucleotide that forms a complex with the fusion protein. In some embodiments, the polynucleotide comprises a tracrRNA. In some embodiments, the polynucleotide that forms a complex with the fusion protein is provided on a vector, e.g., a vector described herein.

In some embodiments, the kit further comprises a template polynucleotide comprising a template sequence for the reverse transcriptase or the DNA polymerase. In some embodiments, the template polynucleotide is provided on a vector, e.g., a vector described herein.

In some embodiments, the kit further comprises a polynucleotide comprising a tracrRNA. In some embodiments, the tracrRNA binds and/or activates the Cas nuclease of the fusion protein. In some embodiments, the polynucleotide comprising a tracrRNA is provided on a vector, e.g., a vector described herein.

In some embodiments, the kit further comprises a DNA polymerase. In some embodiments, the kit further comprises phi29 DNA polymerase, DNA polymerase mu, DNA polymerase delta, or DNA polymerase epsilon. In some embodiments, the kit further comprises a DNA ligase. In some embodiments, the kit further comprises T4 DNA ligase. In some embodiments, the kit further comprises an RNase. In some embodiments, the kit further comprises RNase H.

In some embodiments, the kit further comprises a reaction buffer and/or a storage buffer for the fusion protein, the DNA polymerase, the DNA ligase, and/or the RNase. In some embodiments, the kit further comprises a reagent for performing a DNA cleavage reaction, a reverse transcriptase reaction, a DNA polymerase reaction, a DNA ligase reaction, and/or an RNase reaction. In some embodiments, the reagent comprises ATP, dNTPs, MgC12, Oligo(dT), and/or an RNase inhibitor. In some embodiments, the kit comprises one or more controls, e.g., a control target polynucleotide for the fusion protein. For example, the control target polynucleotide can be designed to be cleaved specifically by the Cas nuclease of the fusion protein with a certain amount of efficiency, thereby calibrating the activity of the Cas nuclease.

In some embodiments, the kit comprises one or more containers. In some embodiments, the kit further comprises a consumable, e.g., a tube, vial, or plate designed to contain samples and/or reagents during one or more steps of the method; a pipette or pipette tips for transferring liquid samples and reagents; a cover and seal for the tube, vial, plate, and/or other consumables used in the method; racks for holding the consumables; labels for identifying samples; and/or instructions for utilizing the kit to provide a site-specific modification at a target sequence in a target polynucleotide as in the methods described herein.

All references cited herein, including patents, patent applications, papers, textbooks and the like, and the references cited therein, to the extent that they are not already, are hereby incorporated herein by reference in their entirety.

### EXAMPLES

### Example 1

In this Example, Cas9 and Cas9 fused to a reverse transcriptase (“PRINS”), along with corresponding guide RNAs, were introduced into cells.

HEK293 cells were plated the day before transfection at a density of 2×10⁵ cells per well of a 12-well plate in 1 mL of complete growth medium (DMEM +10% Fetal Bovine Serum). CRISPR complex components were prepared by combining 0.55 μg of plasmid expressing wild-type Cas9 or PRINS and 0.55 μg of gRNA targeting the AAVS1 locus in 52 μL total volume. Guide RNA sequences for PRINS are described in SEQ ID NOS: 27-28 and target the AAVS1 site to insert the AAGATG sequence. To this mixture, 3.3 μl of FUGENE® HD reagent was added. The solution was mixed carefully by pipetting (approximately 15 times) or by vortexing briefly, then incubated for 5 to 10 minutes at room temperature. To each well containing cells, 50 μL of the complex was added, and the wells were shaken.

Three days after transfection, genomic DNA was extracted, and Amplicon-Seq was performed to amplify the edited sequence. Rational InDel Meta-Analysis (RIMA) was performed on the Amplicon-Seq data to analyze Cas9-induced alterations, as described in Taheri-Ghahfarokhi et al., Nucleic Acids Res 46(16): 8417-8434 (2018).

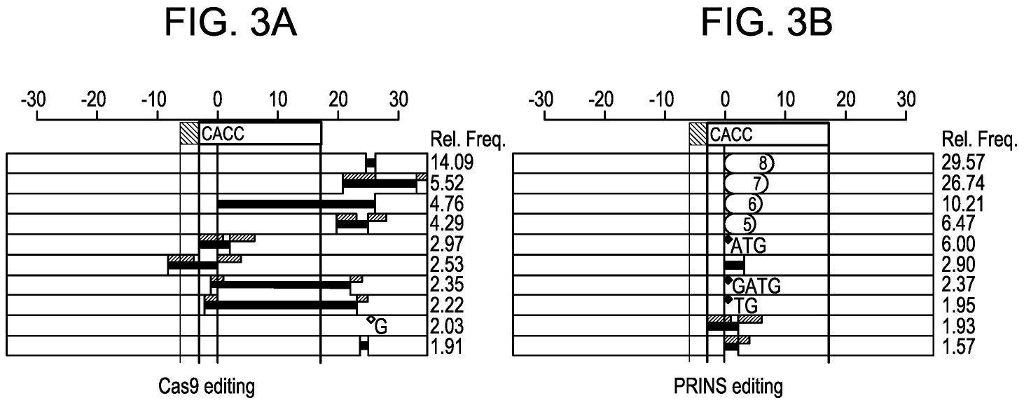

Results are shown in FIGS. 3A and 3B. As shown in FIG. 3A, most of the cells transfected with Cas9 had deletions of variable length. In FIG. 3B, cells transfected with PRINS had a greater number of insertion events (indicated by ovals), and with higher editing efficiency compared with Cas9.

### Example 2

In this Example, Cas9 nickase fused to RT (“PE”) and Cas9 fused to RT (PRINS), along with corresponding prime editing guide RNA (pegRNA) for PE and single primed editing insertion guide RNA (springRNA) for PRINS, both targeting the AAVS1 site as described in Example 1, were introduced into cells. PE and pegRNA are described in Anzalone et al., Nature 576: 149-157 (2019). Briefly, the pegRNA includes a guide sequence complementary to the target sequence and a template sequence that includes the sequence for insertion (AAGATG) flanked by two regions of homology to the target sequence, one of which serving as a primer-binding sequence. The springRNA includes a guide sequence complementary to the target sequence, a template sequence that includes the sequence for insertion (AAGATG), and a primer-binding sequence.

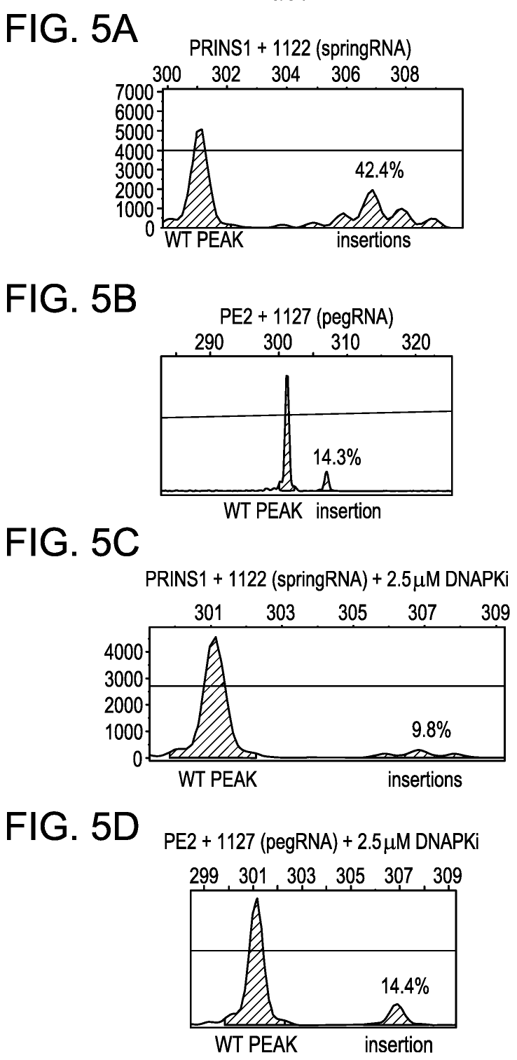

FIGS. 5A and 5B show the insertion frequency of PRINS/springRNA and PE/pegRNA, respectively. Relative editing frequency was determined by Fragment Analysis (see Yang et al., Nucleic Acids Research 43(9): e59 (2015)). PRINS, with 42.4% insertions, is more efficient than PE, which only had 14.3% insertions.

To demonstrate the dependency on NHEJ for PRINS, the same experiment was repeated with 2.5 μM of an inhibitor for a specific DNA-dependent protein kinase (DNAPK) known to be involved in NHEJ. Results in FIGS. 5C and 5D show the insertion frequency of PRINS/springRNA and PE/pegRNA, respectively. No effect of DNAPK inhibition was observed with PE (FIG. 5D), while PRINS had reduced insertion frequency in the presence of the DNAPK inhibitor (FIG. 5C).

### Example 3

In this Example, Cas9 nickase fused to RT (“PE”) Cas9 fused to RT (PRINS) were both tested with pegRNA targeting the AAVS1 site as described in Example 2.

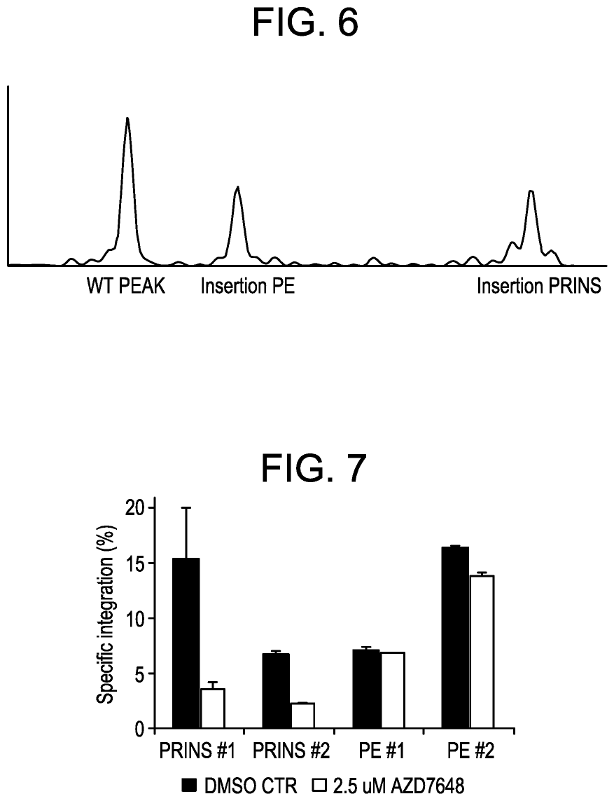

Insertion frequency was analyzed by Fragment Analysis as described in Example 2. Results in FIG. 6 show that pegRNA can promote insertion by PRINS. PRINS can likely utilize pegRNA potentially in a similar manner as PE, as described in Anzalone et al., Nature 576: 149-157 (2019).

### Example 4. Determination of PRINS Editing vs. Prime Editing Mechanisms of Action

In this Example, the mechanism of action of Cas9 fused to RT for PRINS editing was evaluated and compared against the mechanism of Cas9 nickase fused to RT for prime editing. To determine whether PRINS editing and prime editing utilize non-homologous end joining (NHEJ) for DNA repair, an inhibitor of DNA-dependent protein kinase (DNA-PK), a known enzyme in the NHEJ pathway, was introduced.

HEK-T cells were treated with the DNA-PK inhibitor AZD7648 4 hours prior to transfection with the components for PRINS editing and prime editing, as described above for Example 2. The percentage of the specific 6-bp integration (AAGATG) into the AAVS1 locus was assessed using NGS Amplicon-Seq.

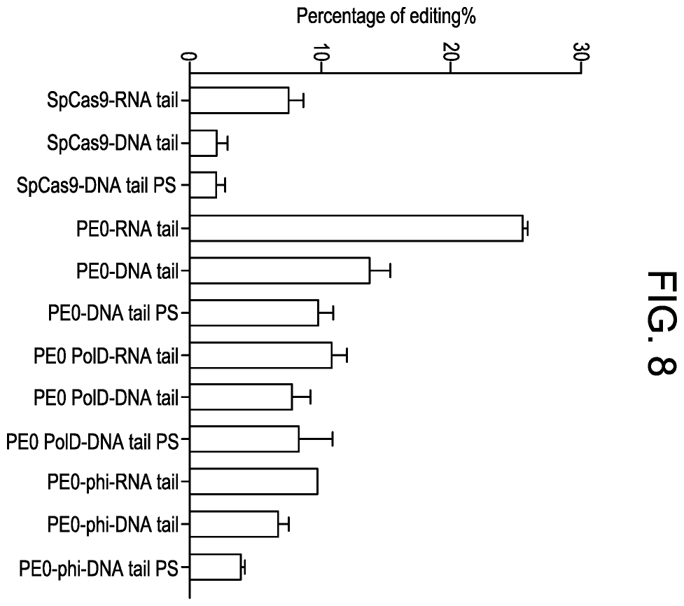

The results are shown in FIG. 7. Bar graphs represent the average of n=2 with standard deviation. The bars labeled as “#1” or “#2” refer to different springRNA (for PRINS editing) or different pegRNA (for prime editing). The data showed that PRINS-mediated integration was strongly reduced by DNA-PK inhibition, while prime editing was relatively unaffected.

### Example 5. Evaluation of DNA and RNA Template Sequences and DNA Polymerase Fusions

In this Example, springRNA was prepared with a DNA template sequence (“DNA tail”) or RNA template sequence (“RNA tail”). Fusions of Cas9+RT (“PE0”), Cas9+DNA Polymerase D (“PE0 PolD”), Cas9+Phi29 DNA polymerase (“PE0 Phi”), and a Cas9 control were tested. Three guide RNAs, one containing an RNA tail (“123RNA MS”) and two containing DNA tails (“123DNA” and “123DNA PS”) were synthesized by Agilent. Sequences are shown in Table 1.

TABLE 1
Guide RNA Sequences

| 123RNA | mG*mU*GGCCCCACUGUGGGGUGGGUUUUAGAGCUAGAAAUA |
|---|---|
| MS | GCAAGUUAAAAUAAGGCUAGUCCGUUAUCAACUUGAAAAAGU |
| | GGGACCGAGUCGGUCCAAGAUGCCCCACAGUGGGGCCACUmA |
| | *mG* (SEQ ID NO: 29) |
| 123DNA | mG*mU*GGCCCCACUGUGGGGUGGGUUUUAGAGCUAGAAAUA |
| | GCAAGUUAAAAUAAGGCUAGUCCGUUAUCAACUUGAAAAAGU |
| | GGGACCGAGUCGGUCCdAdAdGdAdTdGdCdCdCdCdAdCdA |
| | dGdTdGdGdGdGdCdCdAdCdTdAdG (SEQ ID NO: 30) |
| 123DNA | mG*mU*GGCCCCACUGUGGGGUGGGUUUUAGAGCUAGAAAUA |
| PS | GCAAGUUAAAAUAAGGCUAGUCCGUUAUCAACUUGAAAAAGU |
| | GGGACCGAGUCGGUCCdAdAdGdAdTdGdCdCdCdCdAdCdA |
| | dGdTdGdGdGdGdCdCdAdCdTdA*dG* (SEQ ID NO: |
| | 31) |

The fusion proteins were transfected into cells using FUGENE on day 1, and the guide RNAs were transfected with RNAiMAX on day 2.

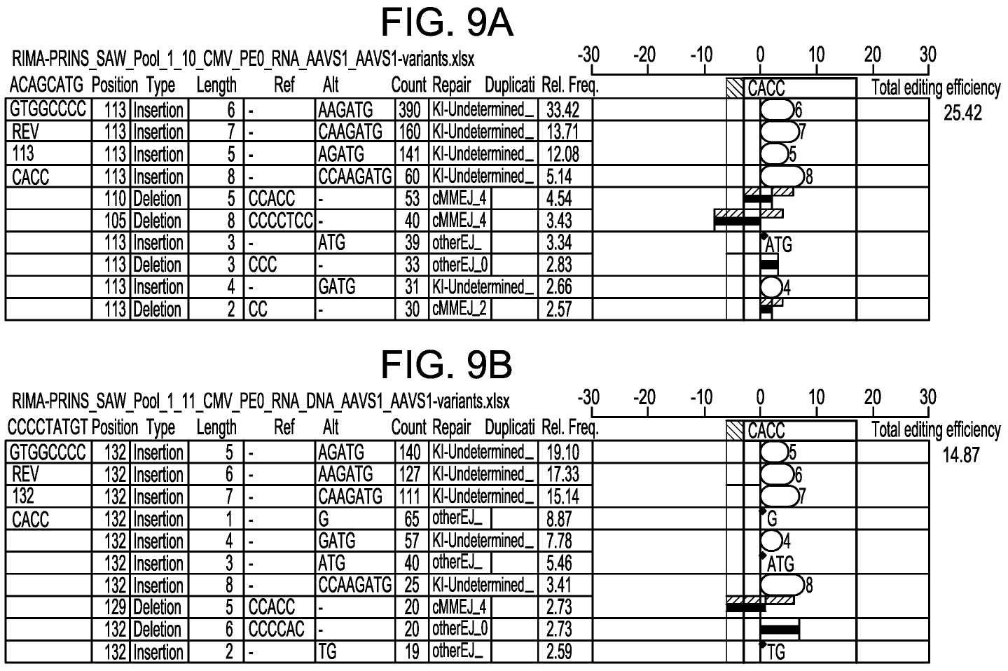

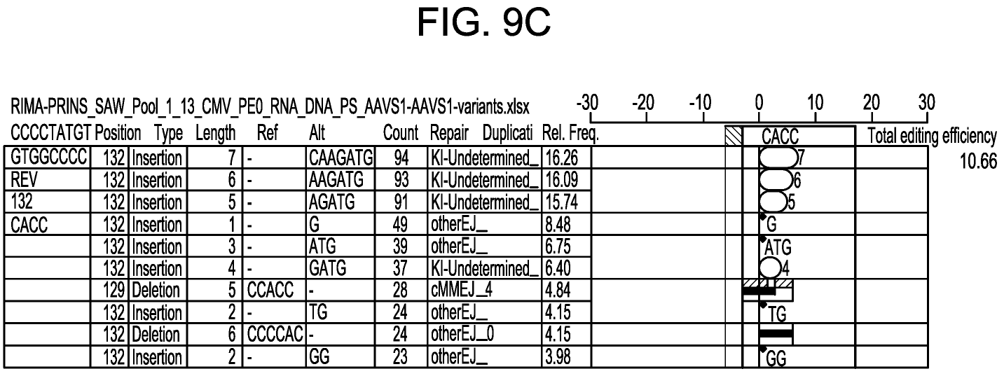

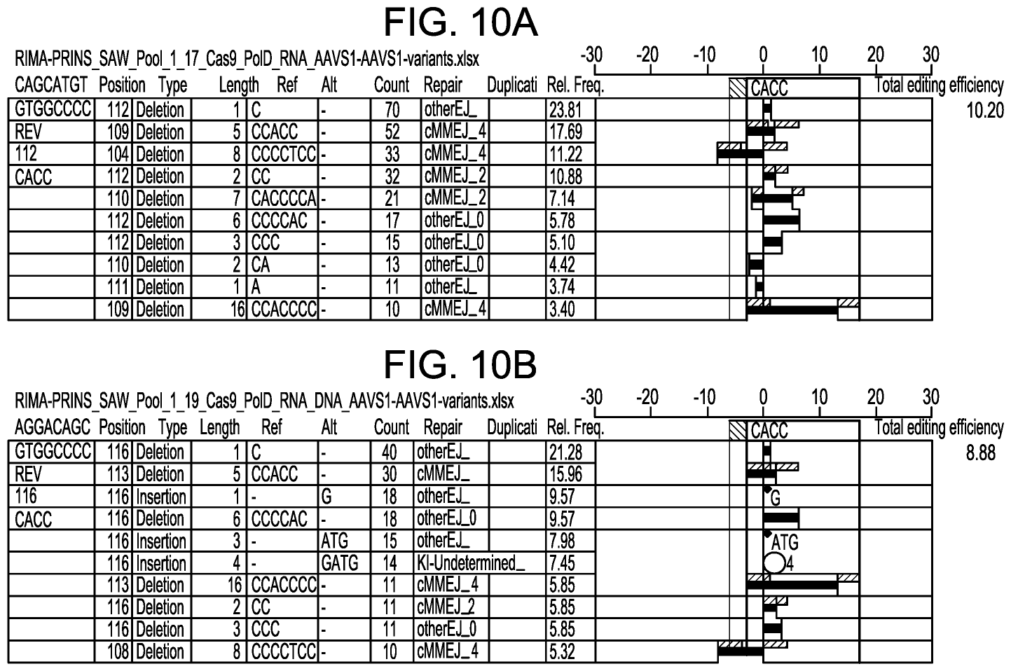

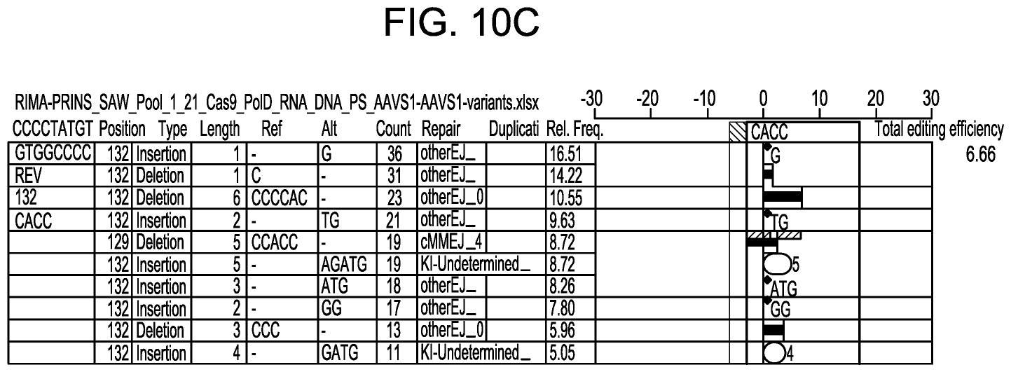

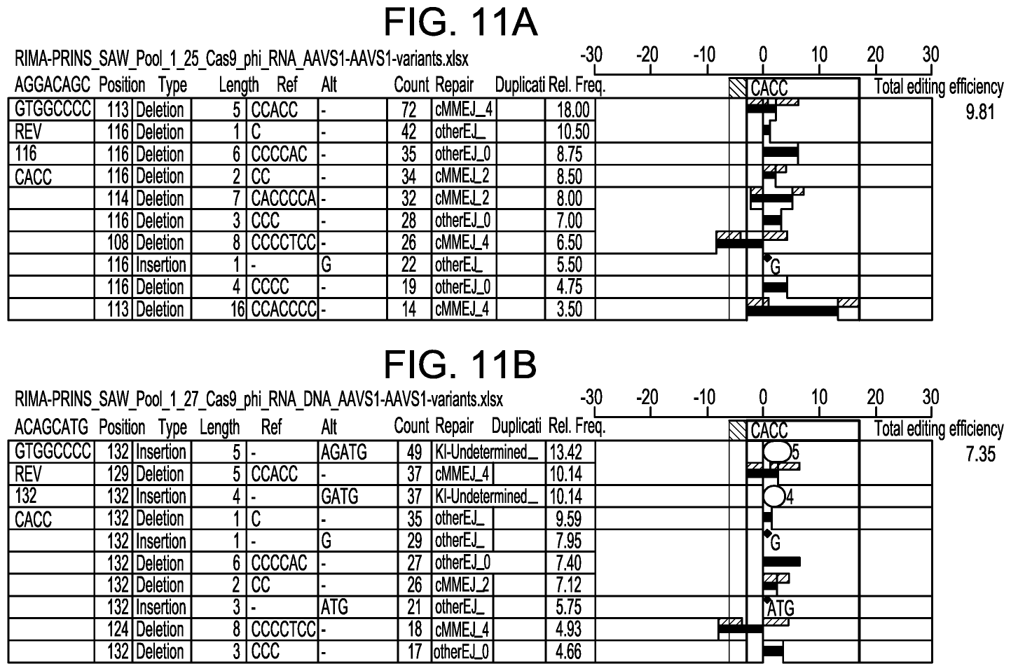

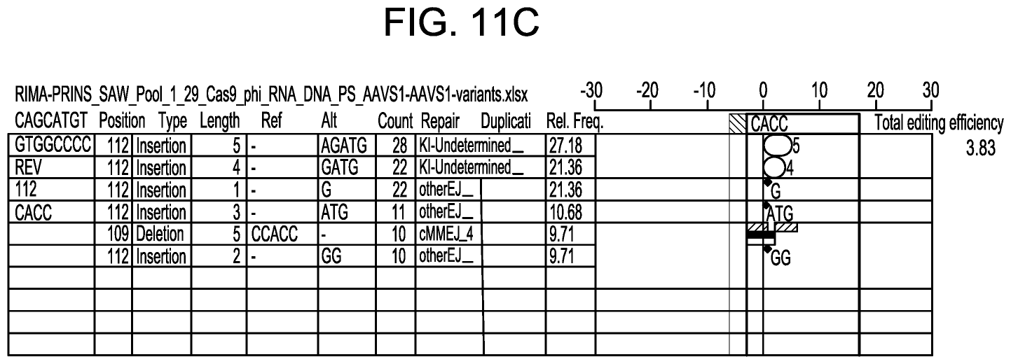

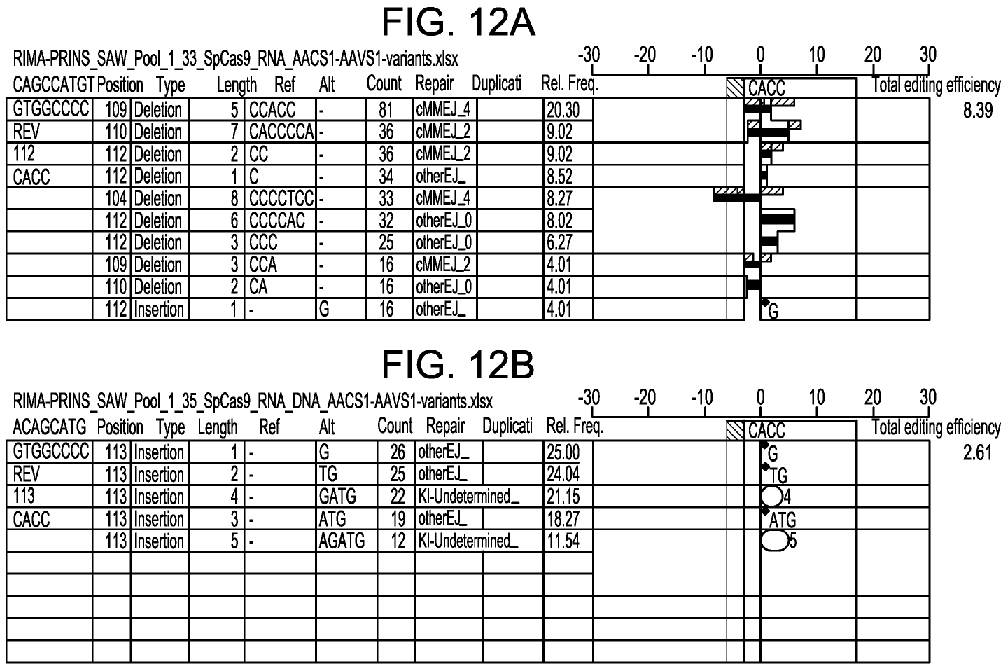

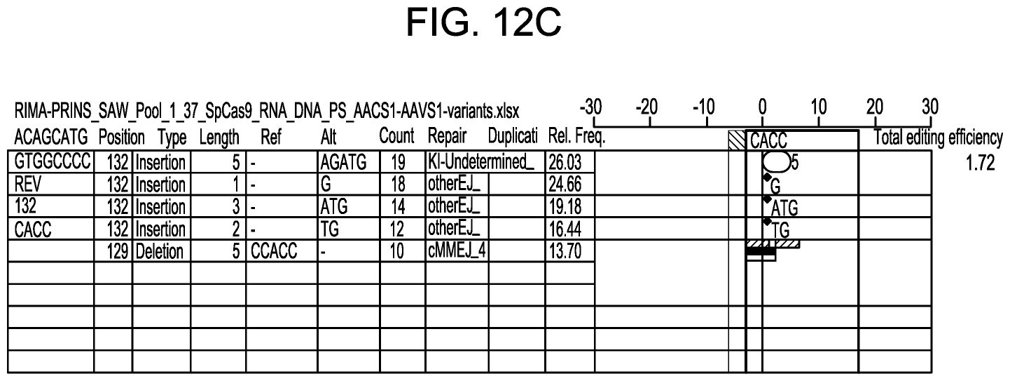

The results are shown in FIGS. 8-12. FIG. 8 shows a summary of the editing efficiency with the different proteins. All fusion proteins achieved higher editing efficiency with the DNA tail sequences compared with Cas9. The top, middle, and bottom panels of FIGS. 9-12 indicate the editing patterns of the indicated protein (PE0, PE0 PolD, PE0 Phi, or Cas9) with 123RNA MS tail, 123DNA tail, or 123DNA PS tail, respectively. Surprisingly, the guide RNA containing DNA tails achieved similar editing pattern using PE0, as shown in FIG. 9. FIGS. 10 and 11 show that DNA polymerases PolD and Phi29 are capable of copying DNA tails, but not RNA tails.

### Example 6. Evaluation of Guide Sequences

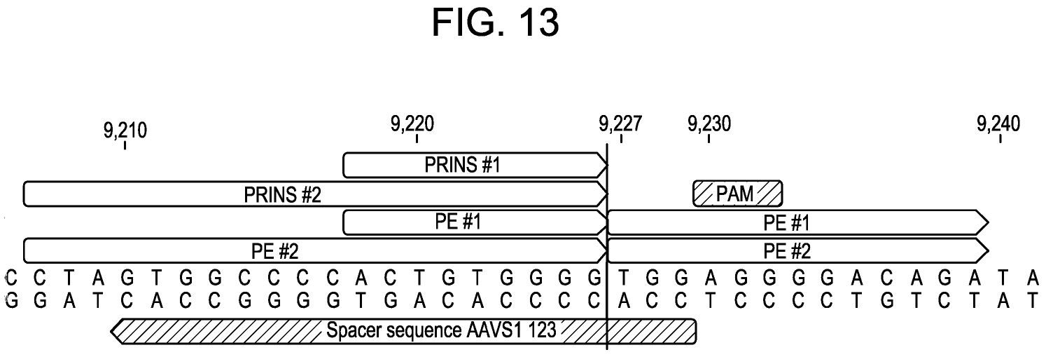

In this Example, different guide sequences were designed and evaluated for their effect on DNA editing by PRINS editing or prime editing. As described in embodiments herein, PRINS editing utilizes a single PRINS guide RNA (springRNA) to target and modify a specific genomic locus. In addition to the spacer and scaffold sequence found in conventional sgRNAs for Cas9 targeting systems, springRNA contains a 3′ extension that includes a primer-binding site (PBS) that hybridizes to the target DNA strand and acts as a primer for reverse transcription. The PBS is followed by the DNA synthesis template containing the desired modification. In comparison, the prime editing guide RNA (pegRNA) includes an additional homology region following the DNA synthesis template, as illustrated in FIG. 13.

To study the effect of different primer designs on PRINS editing and prime editing, HEK-T cells were co-transfected with PRINS editing and prime editing components as described above in Example 2 and in the absence or presence of the DNA-PK inhibitor AZD7648, as described above in Example 4.

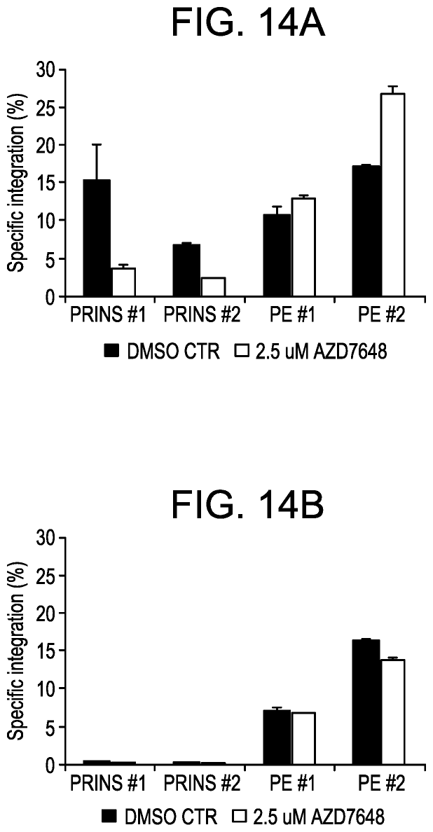

Results are shown in FIGS. 14A and 14B. The data represent the percentage of the specific 6 bp integration (AAGATG) into the AAVS1 locus using PRINS editing (FIG. 14A) and prime editing (FIG. 14B). Bar graphs represent the average of n=2 with standard deviation. The bars labeled as “#1” or “#2” refer to different springRNA and pegRNA designs as shown in FIG. 13. The results demonstrate that PRINS editing functions with both springRNA and pegRNA designs. The combination of PRINS editing with pegRNA and the DNA-PK inhibitor yielded the highest specific editing, outperforming prime editing by two-fold when using the same pegRNA. Prime editing produced detectable modifications with pegRNA, but did not produce any detectable modifications with springRNA.

### Example 7. Evaluation of PRINS Editing Toxicity

In this Example, the toxicity of PRINS editing compared to Cas9 editing was evaluated by determining the number of large deletions induced after generation of the double-stranded break.

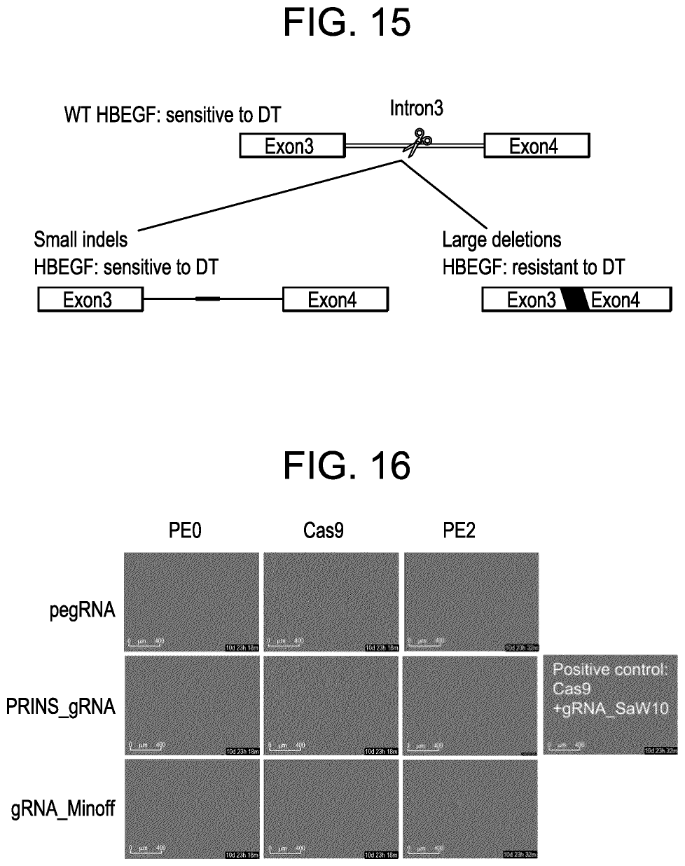

A diphtheria toxin (DT) selection system (e.g., as described in U.S. Provisional Application No. 62/833,404 filed Apr. 12, 2020 and PCT/EP2020/060250) was used to assess the amount of large deletions. FIG. 15 illustrates a schematic of the experimental design. Briefly, an intron of HbEGF, the DT receptor, was selected as the PRINS editing or Cas9 editing target. Only a bi-allelic large deletion will provide the cell with DT resistance, and thus, cell survival after DT treatment is indicative of the amount of large deletions.

Cells were transfected with a Cas9-RT fusion (PRINS editing, “PE0”), Cas9, or Cas9 nickase-RT fusion (prime editing, “PE2”) and three different guide RNAs. Results in FIG. 16 show that after transfection of the same number of cells with the same amount of DNA, the PE0 plate shows fewer cells relative to the Cas9 plate, indicating a lower number of large deletions with PRINS editing. The number of large deletions by PRINS editing is comparable to that of prime editing with PE2.

### Example 8. Evaluation of Exogenous Template Polynucleotide

In this Example, the addition of an exogenous template polynucleotide not fused to the guide RNA for PRINS editing or prime editing was evaluated.

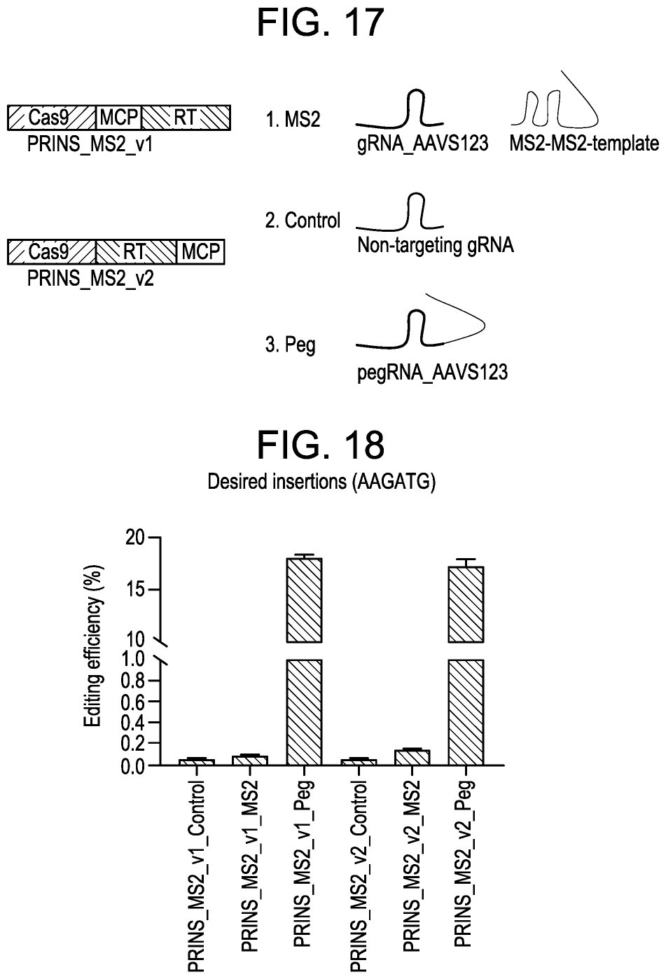

A schematic of the experimental design is illustrated in FIG. 17. An MCP domain, which binds to MS2 aptamers, was fused to the Cas9-RT protein used in PRINS editing, either in between the Cas9 and RT (“PRINS_MS2_v1”) or downstream of the RT (“PRINS_MS2_v2”). The template for reverse transcription was fused to MS2 aptamers instead of to the guide RNA. PRINS_MS2, MS2-RT template, and target gRNA were co-transfected into HEK-T cells and tested for targeted insertions. Control gRNA and a RT template fused to gRNA served as negative and positive controls, respectively.

Results in FIG. 18 show that a DNA sequence was successfully copied and inserted specifically from MS2-RT template by PRINS editing, even though the editing efficiency is lower than PRINS editing using a RT template fused to gRNA.

### Example 9. Evaluation of Cas12 Fusions for PRINS Editing

In this Example, a Cas12-RT fusion protein was evaluated for PRINS editing and prime editing ability.

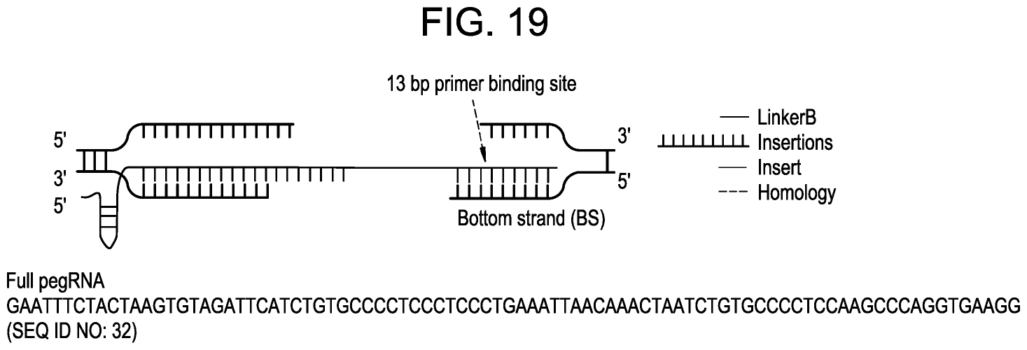

RT was fused to LbCas12 (also known as LbCpf1). Guide RNAs were designed for PRINS editing (springRNA) and prime editing (pegRNA) at the EMX1 and DNMT1 sites. An exemplary guide RNA targeting EMX1 is shown in FIG. 19 and included the following sequence, with single underline indicating the insertion sequence and the double underline indicating the homology sequence:

(SEQ ID NO: 31)
GAATTTCTACTAAGTGTAGATTCATCTGTGCCCCTCCCTCCCTGAAATTA
ACAAACTAATCTGTGCCCCTCCAAGCCCAGGTGAAGG

The insertions at the EMX1 site using the above guide RNA were determined, as shown in Table 2.

TABLE 2
Insertions at EMX1 Site

| | | | | Total |
|---|---|---|---|---|
| | | | Alternatives to underlined | Frequency |
| Position | Type | Length | portions in SEQ ID NO: 31 | (%) |
| 124 | Insertion | 30 | AAATTAACAAACTAATCTGTGCCCCT | 10.25 |
| | | | CCAA (SEQ ID NO: 32) | |
| 123 | Insertion | 30 | GAAATTAACAAACTAATCTGTGCCCC | 0.11 |
| | | | TCCA (SEQ ID NO: 33) | |
| 119 | Insertion | 23 | GAAATTAACAAACTAATCTGTGC | 0.09 |
| | | | (SEQ ID NO: 34) | |

The types of mutations were determined, as shown in Table 3.

TABLE 3
Types of Mutations

| Efficiency | No. Reads | % |
|---|---|---|
| Mutated % | 34908 | 27.16 |
| In-Frame % | 9747 | 27.92 |
| Out-of-frame % | 25161 | 72.08 |

The results in Tables 2 and 3 show that a DNA sequence was successfully copied and inserted specifically by a Cas12-RT fusion protein using PRINS editing. Overall editing efficiency was approximately 0.25%.

### Example 10. PRINS Editing with Cas9-DNA Polymerase Fusion

Cas9 fused to a DNA polymerase was evaluated for PRINS editing. DNA polymerases have been reported to exhibit reverse transcriptase activity in vitro and in vivo (see, e.g., Ricchetti et al., EMBO J. 12(2):387-396 (1993)). A plasmid expressing either Cas9, Cas9-RT fusion (“PE0”), or Cas9 fused with a DNA polymerases as indicated below, was transfected into HEK293T cells along with a plasmid expressing a single primed editing insertion guide RNA (springRNA) targeting the AAVS1 locus. The Cas9-DNA polymerase fusion contained the following DNA polymerase constructs:

Cas9-Klenow exo+: Codon-optimized Klenow fragment of E. coli DNA Polymerase I;

Cas9-Klenow exo−: Codon-optimized Klenow fragment of E. coli DNA Polymerase I with D355A and E357A mutations, which abolish the 3′→5′ exonuclease activity of the DNA polymerase;

Cas9-REV3: A catalytically active truncation of the human REV3 polymerase, which was identified to have increased stability and higher expression level as compared to full length REV3 (denoted as REV TR5; see Lee et al., PNAS (2014), doi: 10.1073/pnas.1324001111).

The cells were harvested 72 hours post-transfection. Genomic DNA was extracted, and the AAVS1 locus was amplified by PCR and sequenced using the Illumina sequencing platform.

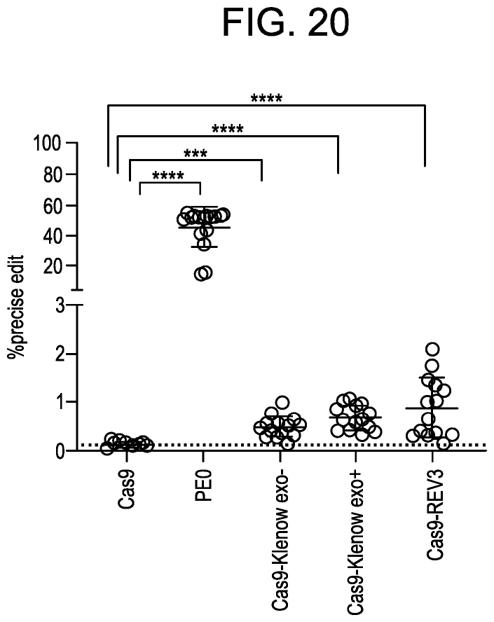

Results in FIG. 20 show that the three Cas9-DNA polymerase fusion proteins were capable of PRINS editing.

### Example 11. PRINS Editing with Cas9-DNA Polymerase Fusion and Chimeric springRNA

Chimeric springRNAs were evaluated in PRINS editing with Cas9, PE0, and Cas9-DNA polymerase fusion proteins. HEK293T cells were transfected, using EUGENE® HD, with plasmids expressing Cas9, PE0, or the three Cas9-DNA polymerase fusion proteins described in Example 10. After 24 hours, the cells were further transfected, using LIPOFECTAMINE™ RNAiMAX, with 2 pmol of one of the following synthetic springRNA:

springRNA—all RNA nucleotides; the sequence contains the guide RNA sequence; tracrRNA scaffold for binding Cas9; and 6-nucleotide insert sequence (“AATATG”) and primer binding site (PBS) at the 3′ of the springRNA;

Chimeric springRNA DiHP—same sequence as above for springRNA, all RNA nucleotides except that the insert sequence and 10 nucleotides of the PBS are deoxyribonucleotides;

Chimeric springRNA DiRP—same sequence as above for springRNA, all RNA nucleotides except that the insert sequence is dexoyribonucleotides.

The cells were harvested 48 hours post-transfection. Genomic DNA was extracted, and the AAVS1 locus was amplified by PCR and sequenced using the Illumina sequencing platform.

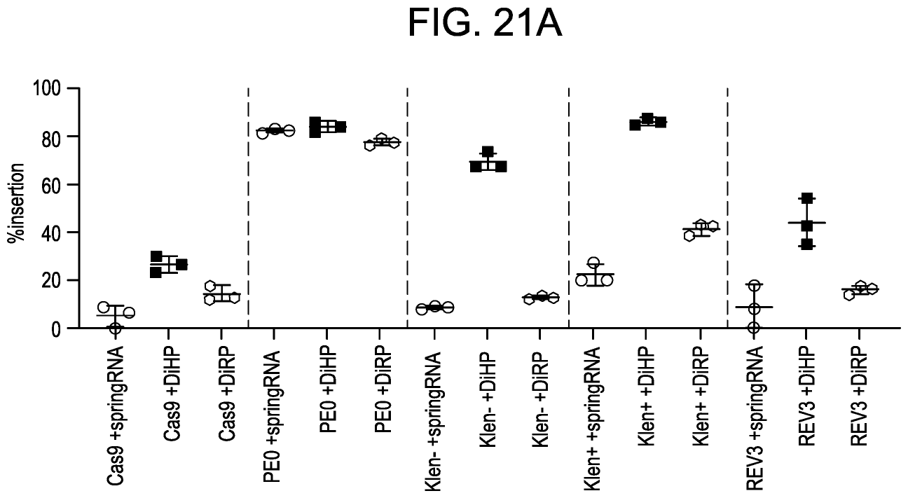

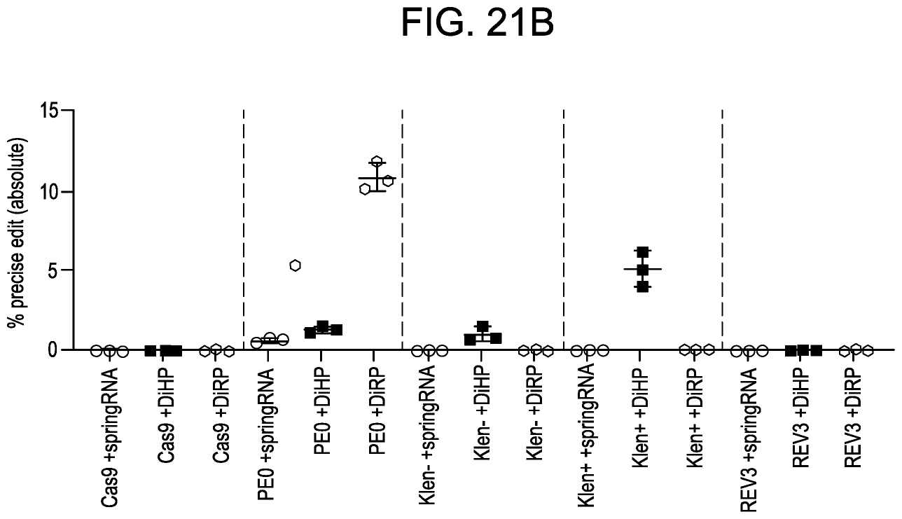

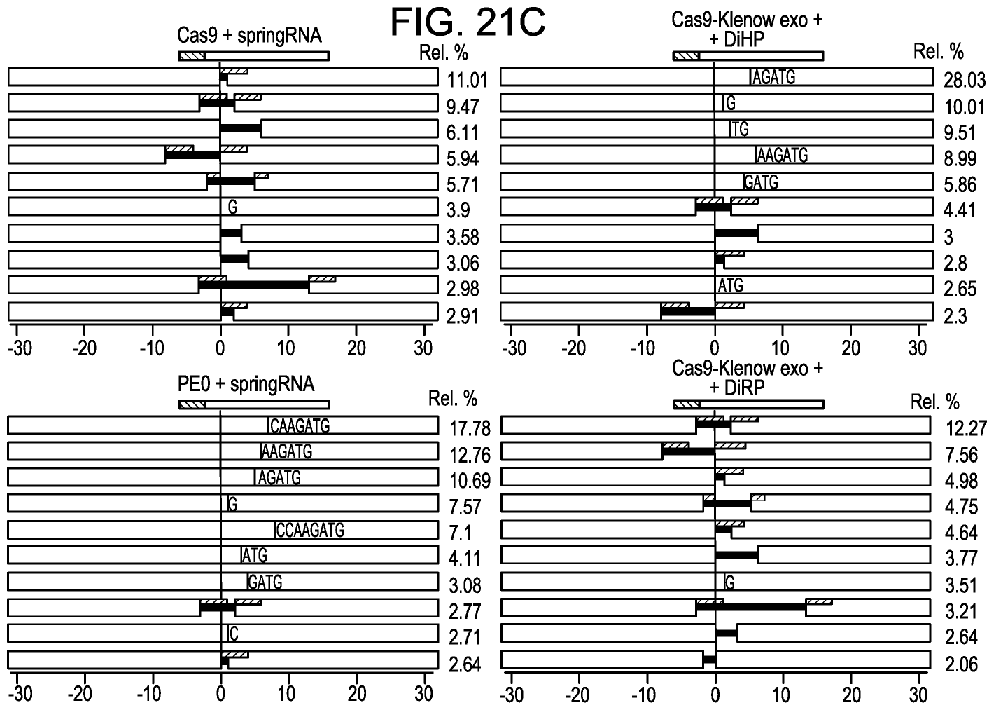

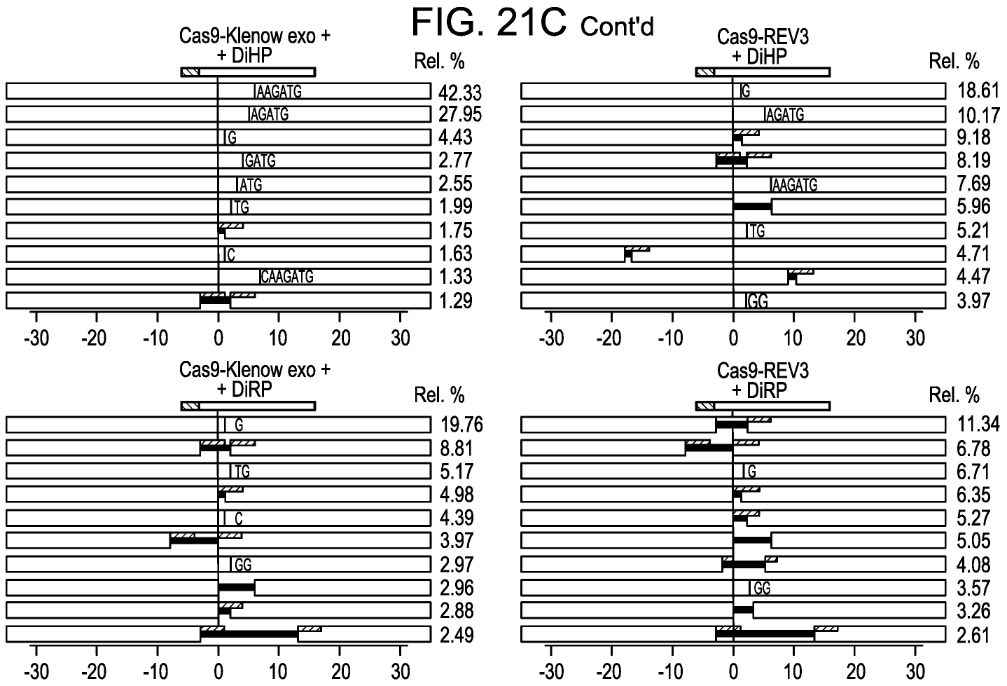

Results in FIGS. 21A-C show that the Cas9-DNA polymerase fusion protein was capable of PRINS editing with efficiency comparable to PE0 when using chimeric, DNA-containing springRNAs.

### Example 12. PRINS Editing with Cas9-DNA Polymerase Fusion and Modified springRNA

Various springRNAs with chemical modifications were evaluated in PRINS editing. HEK293T cells were transfected, using FUGENE® HD, with plasmids expressing Cas9 or PE0. After 24 hours, the cells were further transfected, using LIPOFECTAMINE™ RNAiMAX, with 2 pmol of one of the following springRNA:

springRNA—all RNA nucleotides; the sequence contains the guide RNA sequence; tracrRNA scaffold for binding Cas9; and 6-nucleotide insert sequence (“AATATG”) and primer binding site (PBS) at the 3′ of the springRNA;

springRNA with abasic site—same sequence as above for springRNA, all RNA nucleotides except that the third nucleotide in the insert sequence is replaced by a dSpacer nucleotide 1′2′-dideoxyribose (abasic site);

springRNA with TEG linker—same sequence as above for springRNA, all RNA nucleotides except that the third nucleotide in the insert sequence is covalently attached to a triethylene glycol (TEG).

The cells were harvested 48 hours post-transfection. Genomic DNA was extracted, and the AAVS1 locus was amplified by PCR and sequenced using the Illumina sequencing platform.

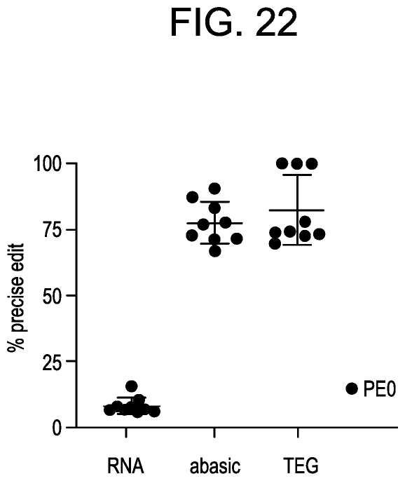

Results in FIG. 22 show that the chemically modified springRNAs were capable of preventing overextension of the insert and increase the precision of mutagenesis.

### Example 13. PRINS Editing with Cas9-DNA Ligase Fusion

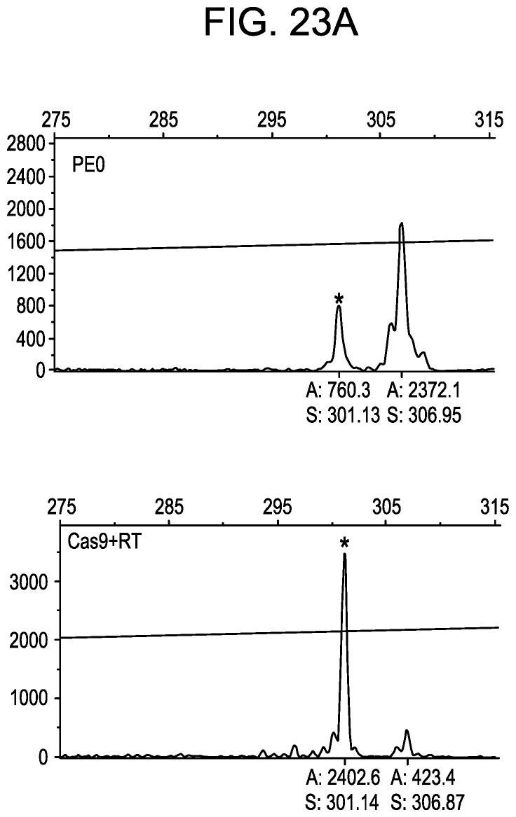

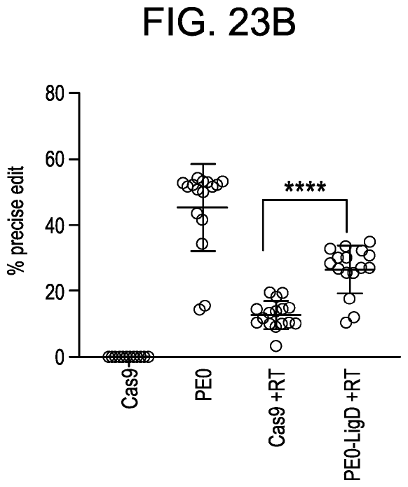

Cells were transfected with Cas9 and RT on separate expression plasmids and a plasmid containing springRNA and evaluated for PRINS editing. As shown in FIG. 23A, PRINS editing still occurred with co-expression of Cas9 and RT proteins (asterisk denotes wild-type sequence).

Cas9 fused to a DNA ligase was then evaluated for PRINS editing. Cas9 was fused to Mycobacterium tuberculosis LigD, which is a DNA ligase involved in non-homologous end joining of DNA breaks (“Cas9-LigD”). A plasmid expressing the Cas9-LigD fusion protein was co-transfected with plasmids expressing RT and a springRNA plasmid and evaluated for PRINS editing.

Results in FIG. 23B shows that co-transfection of the Cas9-LigD fusion protein and RT had improved insertion of the desired sequence as compared to co-expression of Cas9 and RT.

### Example 14. Mismatches of Insert and PBS in springRNA

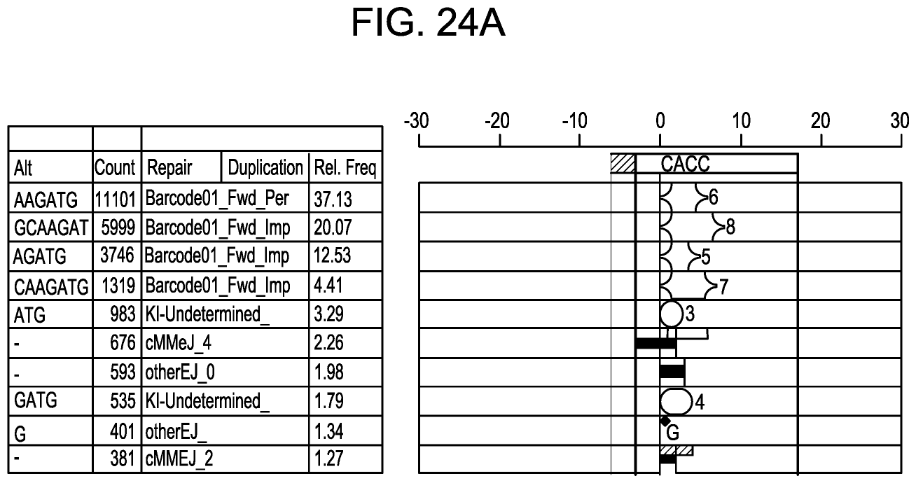

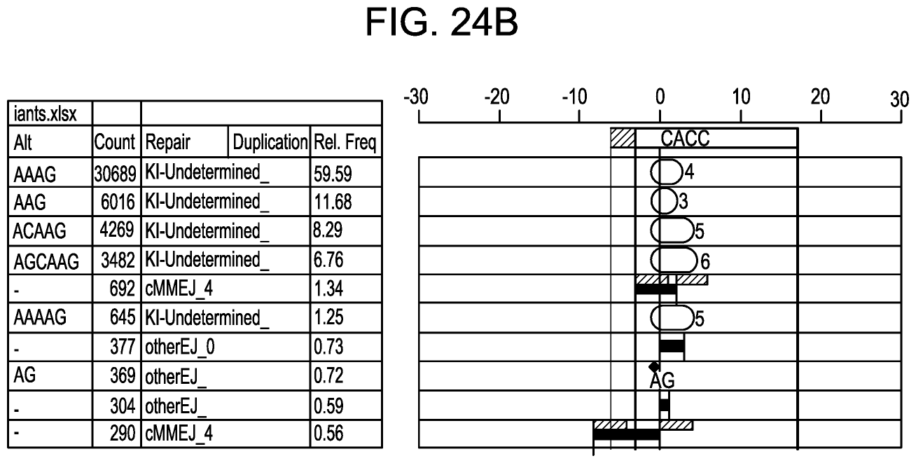

Mismatches were introduced in the primer binding site (PBS) of the springRNA in order to reduce homology between the 5′ and 3′ of the springRNA, which resulted in two mismatches between the 3′ end of the target DNA strand annealed to the PBS. Typically, DNA is primed less efficiency when a 3′ mismatch with a template is present. Surprisingly, as shown in FIGS. 24A-24B, insertion of the 4 bp insert sequence (originally 6 bp sequence minus the 2 bp mismatch) was more efficient than insertion of the fully complementary 6 bp insert. The 4 bp insertion with 2 bp mismatch had a relative insertion efficiency of 59.59% (FIG. 24B), while the 6 bp insertion with no mismatch had a relative insertion efficiency of 37.13% (FIG. 24A).

### Example 15. Effect of DNA Repair Pathway on PRINS and Prime Editing

The PRINS editing efficiency of PE0 with springRNA and the prime editing efficiency of PE0 with pegRNA were evaluated in cell lines partially deficient in the following DNA repair genes: PRKDC (also known as DNAPK), LIG4, TP53BP1, PARP1, POLQ, LIG3, and ATM. The cells were also cultured in the presence of absence of a DNAPK inhibitor.

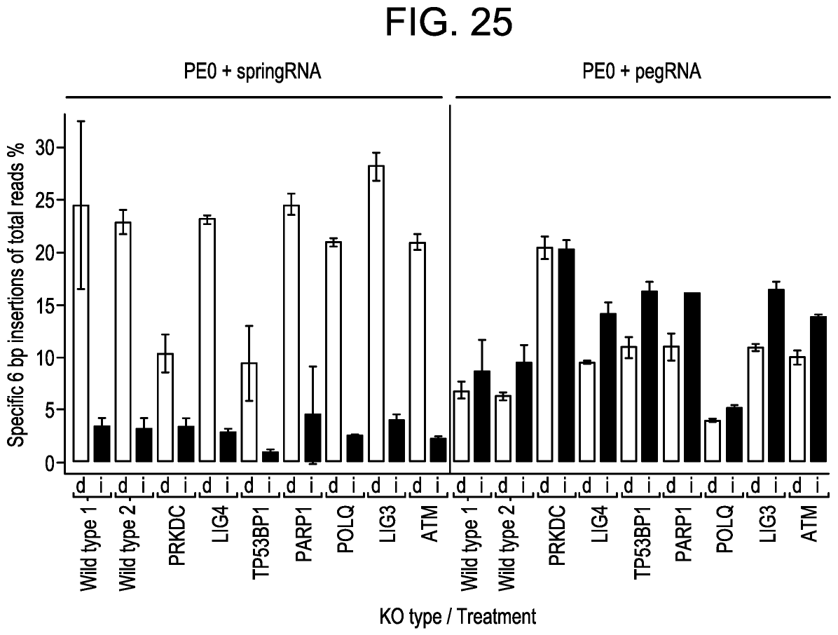

Results are shown in FIG. 25 and indicate that PRINS editing is dependent on NHEJ pathway enzymes such as PRKDC and TP53BP1, as deletion of these genes or inhibition of the PRKDC protein resulted in lower PRINS efficiency. FIG. 25 also shows that prime editing with PE0 and pegRNA had an inverse correlation with NHEJ enzymes, as inhibition or deletion of PRKDC, LIG4, or TP53BP1 resulted in a higher insertion efficiency.

### Example 16. Evaluation of Type II-B Cas9 Fusions for PRINS Editing

A fusion protein comprising a type II-B Cas9 protein, the Cas9 from the sequenced gut metagenome MH0245_GL0161830.1 (MHCas9) that generates cohesive ends (“overhangs”), and MMLV reverse transcriptase. SpringRNA was designed for binding to the MHCas9 and containing a six-nucleotide insert sequence targeting the AAVS1 locus as described for Example 10. HEK293T cells were transfected, and the genomic DNA was extracted, and Amplicon-Seq was used to detect the targeted insertion.

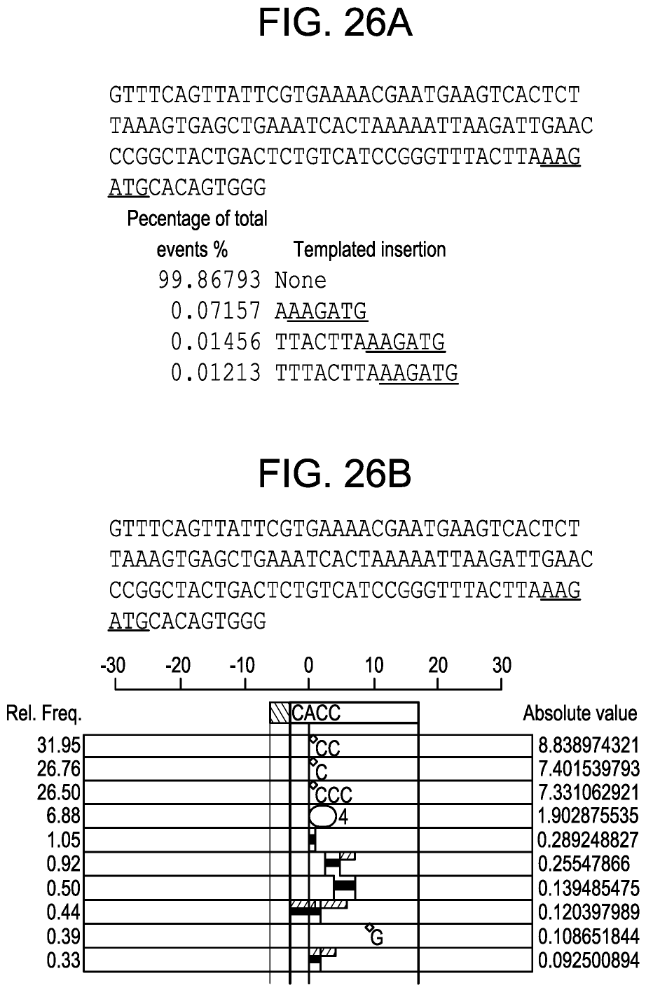

Results in FIG. 26A show that the MHCas9-RT fusion protein successfully performed PRINS-mediated insertion at the target locus. The most efficient insert had an insertion frequency of 0.072%. FIG. 26B shows the ten most frequent editing events by MHCas9-RT. The RT not only mediated insertion of the insert sequence but also extended the overhang sequences (CCC) generated by the MHCas9, as indicated by the three most frequent editing events.

### Example 17. Targeted Insertions and Deletions with MHCas9-RT Fusion

The Cas9-RT fusion protein (“PE0”) as described in the previous Examples was evaluated for the ability to perform targeted insertions and deletions using pegRNA. In contrast with prime editing, which utilizes a Cas9 nickase-RT fusion and pegRNA, PE0 with pegRNA introduces a double-stranded DNA break and is therefore repaired by double-stranded DNA break repair pathways that are not involved in prime editing. PegRNA and prime editing are described in Example 2 and Anzalone et al., Nature 576: 149-157 (2019).

HEK293T cells were transfected with plasmids expressing MHCas9-RT and pegRNA targeting the AAVS1 site, as described in the previous Examples. Two different pegRNA constructs were tested: 1) a construct to provide a 1 nucleotide deletion; and 2) a construct to produce an A to G substitution at the PAM-3 site. After transfection, genomic DNA was extracted and processed by NGS as described in the previous Examples.

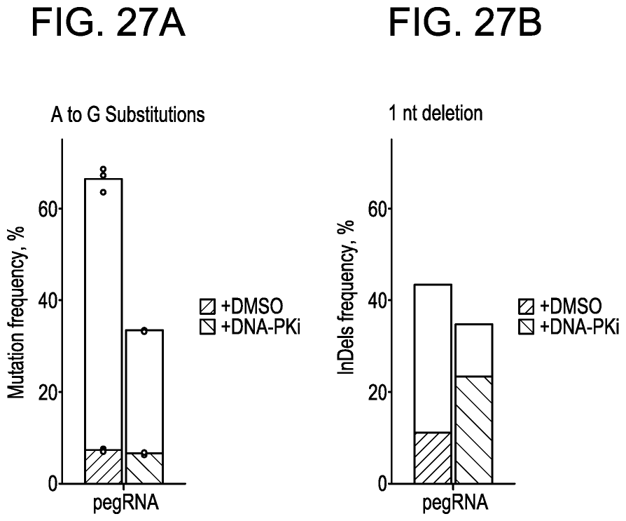

Results in FIGS. 27A (A to G substitution) and 27B (1 nucleotide deletion) demonstrate that PE0 with pegRNA is capable of inducing substitution/insertions and deletions. The dark grey portions in the bar graphs of FIGS. 27A and 27B represent the desired mutation, and the light grey portions represent undesired mutations. The experiment was also performed in the presence of a DNAPK inhibitor (DNAPKi) increased the percentage of the desired mutation relative to undesired mutations.

### SEQUENCES

Sequences of various polynucleotides and polypeptides are provided herein.

Amino acid sequence of a Cas9 nuclease
(SEQ ID NO: 1)
DKKYSIGLDIGTNSVGWAVITDEYKVPSKKFKVLGNTDRHSIKKNLIGALLFDSGETAEATRLK
RTARRRYTRRKNRICYLQEIFSNEMAKVDDSFFHRLEESFLVEEDKKHERHPIFGNIVDEVAYH
EKYPTIYHLRKKLVDSTDKADLRLIYLALAHMIKFRGHFLIEGDLNPDNSDVDKLNIQLVQTYN
QLFEENPINASGVDAKAILSARLSKSRRLENLIAQLPGEKKNGLFGNLIALSLGLTPNFKSNFD
LAEDAKLQLSKDTYDDDLDNLLAQIGDQYADLFLAAKNLSDAILLSDILRVNTEITKAPLSASM
IKRYDEHHQDLTLLKALVROQLPEKYKEIFFDQSKNGYAGYIDGGASQEEFYKFIKPILEKMDG
TEELLVKLNREDLLRKQRTFDNGSIPHQIHLGELHAILRRQEDFYPFLKDNREKIEKILTFRIP
YYVGPLARGNSRFAWMTRKSEETITPWNFEEVVDKGASAQSFIERMTNFDKNLPNEKVLPKHSL
LYEYFTVYNELTKVKYVTEGMRKPAFLSGEQKKAIVDLLFKTNRKVTVKQLKEDYFKKIECFDS
VEISGVEDRFNASLGTYHDLLKIIKDKDFLDNEENEDILEDIVLTLTLFEDREMIEERLKTYAH
LFDDKVMKQLKRRRYTGWGRLSRKLINGIRDKQSGKTILDFLKSDGFANRNFMQLIHDDSLTFK
EDIQKAQVSGQGDSLHEHIANLAGSPAIKKGILQTVKVVDELVKVMGRHKPENIVIEMARENQT
TQKGQKNSRERMKRIEEGIKELGSQILKEHPVENTQLQNEKLYLYYLQNGRDMYVDQELDINRL
SDYDVDHIVPQSFLKDDSIDNKVLTRSDKNRGKSDNVPSEEVVKKMKNYWRQLLNAKLITQRKF
DNLTKAERGGLSELDKAGFIKRQLVETRQITKHVAQILDSRMNTKYDENDKLIREVKVITLKSK
LVSDFRKDFQFYKVREINNYHHAHDAYLNAVVGTALIKKYPKLESEFVYGDYKVYDVRKMIAKS
EQEIGKATAKYFFYSNIMNFFKTEITLANGEIRKRPLIETNGETGEIVWDKGRDFATVRKVLSM
PQVNIVKKTEVQTGGFSKESILPKRNSDKLIARKKDWDPKKYGGFDSPTVAYSVLVVAKVEKGK
SKKLKSVKELLGITIMERSSFEKNPIDFLEAKGYKEVKKDLIIKLPKYSLFELENGRKRMLASA
GELQKGNELALPSKYVNFLYLASHYEKLKGSPEDNEQKQLFVEQHKHYLDEITEQISEFSKRVI
LADANLDKVLSAYNKHRDKPIREQAENIIHLFTLTNLGAPAAFKYFDTTIDRKRYTSTKEVLDA
TLIHQSITGLYETRIDLSQLGGD
Amino acid sequence of a Cas12 nuclease (LbCas12a)
(SEQ ID NO: 29)
MSKLEKFTNCYSLSKTLRFKAIPVGKTQENIDNKRLLVEDEKRAEDYKGVKKLLDRYYLSFIND
VLHSIKLKNLNNYISLFRKKTRTEKENKELENLEINLRKEIAKAFKGNEGYKSLFKKDIIETIL
PEFLDDKDEIALVNSFNGFTTAFTGFFDNRENMFSEEAKSTSIAFRCINENLTRYISNMDIFEK
VDAIFDKHEVQEIKEKILNSDYDVEDFFEGEFFNFVLTQEGIDVYNAIIGGFVTESGEKIKGLN
EYINLYNQKTKQKLPKFKPLYKQVLSDRESLSFYGEGYTSDEEVLEVFRNTLNKNSEIFSSIKK
RRKSFKKIGSFSLEQLQEYADADLSVVEKLKEIIIQKVDEIYKVYGSSEKLFDADFVLEKSLKK
NDAVVAIMKDLLDSVKSFENYIKAFFGEGKETNRDESFYGDFVLAYDILLKVDHIYDAIRNYVT
QKPYSKDKFKLYFQNPQFMGGWDKDKETDYRATILRYGSKYYLAIMDKKYAKCLQKIDKDDVNG
NYEKINYKLLPGPNKMLPKVFFSKKWMAYYNPSEDIQKIYKNGTFKKGDMFNLNDCHKLIDFFK
DSISRYPKWSNAYDFNFSETEKYKDIAGFYREVEEQGYKVSFESASKKEVDKLVEEGKLYMFQI
YNKDFSDKSHGTPNLHTMYFKLLFDENNHGQIRLSGGAELFMRRASLKKEELVVHPANSPIANK
NPDNPKKTTTLSYDVYKDKRFSEDQYELHIPIAINKCPKNIFKINTEVRVLLKHDDNPYVIGID
RGERNLLYIVVVDGKGNIVEQYSLNEIINNFNGIRIKTDYHSLLDKKEKERFEARQNWTSIENI
KELKAGYISQVVHKICELVEKYDAVIALEDLNSGFKNSRVKVEKQVYQKFEKMLIDKLNYMVDK
KSNPCATGGALKGYQITNKFESFKSMSTQNGFIFYIPAWLTSKIDPSTGFVNLLKTKYTSIADS
KKFISSFDRIMYVPEEDLFEFALDYKNFSRTDADYIKKWKLYSYGNRIRIFRNPKKNNVFDWEE
VCLTSAYKELFNKYGINYQQGDIRALLCEQSDKAFYSSFMALMSLMLQMRNSITGRTDVDFLIS
PVKNSDGIFYDSRNYEAQENAILPKNADANGAYNIARKVLWAIGQFKKAEDEKLDKVKIAISNK
EWLEYAQTSVKH
Amino Acid Sequence of a Cas14 nuclease (Cas14a1)
(SEQ ID NO: 30)
MEVQKTVMKTLSLRILRPLYSQEIEKEIKEEKERRKQAGGTGELDGGFYKKLEKKHSEMFSFDR
LNLLLNQLQREIAKVYNHAISELYIATIAQGNKSNKHYISSIVYNRAYGYFYNAYIALGICSKV
EANFRSNELLTQQSALPTAKSDNFPIVLHKQKGAEGEDGGFRISTEGSDLIFEIPIPFYEYNGE
NRKEPYKWVKKGGQKPVLKLILSTFRRQRNKGWAKDEGTDAEIRKVTEGKYQVSQIEINRGKKL
GEHQKWFANFSIEQPIYERKPNRSIVGGLDVGIRSPLVCAINNSFSRYSVDSNDVFKFSKQVFA
FRRRLLSKNSLKRKGHGAAHKLEPITEMTEKNDKFRKKIIERWAKEVTNFFVKNQVGIVQIEDL
STMKDREDHFFNQYLRGFWPYYQMQTLIENKLKEYGIEVKRVQAKYTSQLCSNPNCRYWNNYFN
FEYRKVNKFPKFKCEKCNLEISADYNAARNLSTPDIEKFVAKATKGINLPEK
Amino acid sequence of MMLV reverse transcriptase
(SEQ ID NO: 2)
TLNIEDEYRLHETSKEPDVSLGSTWLSDFPQAWAETGGMGLAVRQAPLIIPLKATSTPVSIKQY
PMSQEARLGIKPHIQRLLDQGILVPCQSPWNTPLLPVKKPGTNDYRPVQDLREVNKRVEDIHPT
VPNPYNLLSGLPPSHQWYTVLDLKDAFFCLRLHPTSQPLFAFEWRDPEMGISGQLTWTRLPQGF
KNSPTLFNEALHRDLADFRIQHPDLILLQYVDDLLLAATSELDCQQGTRALLQTLGNLGYRASA
KKAQICQKQVKYLGYLLKEGQRWLTEARKETVMGQPTPKTPRQLREFLGKAGFCRLFIPGFAEM
AAPLYPLTKPGTLFNWGPDQQKAYQEIKQALLTAPALGLPDLTKPFELFVDEKQGYAKGVLTQK
LGPWRRPVAYLSKKLDPVAAGWPPCLRMVAAIAVLTKDAGKLTMGQPLVILAPHAVEALVKQPP
DRWLSNARMTHYQALLLDTDRVQFGPVVALNPATLLPLPEEGLQHNCLDILAEAHGTRPDLTDQ
PLPDADHTWYTDGSSLLQEGORKAGAAVTTETEVIWAKALPAGTSAQRAELIALTQALKMAEGK
KLNVYTDSRYAFATAHIHGEIYRRRGWLTSEGKEIKNKDEILALLKALFLPKRLSIIHCPGHQK
GHSAEARGNRMADQAARKAAITETPDTSTLLIENSSPSGGSKRTADGSEFE
Amino acid sequence of R2 reverse transcriptase
(SEQ ID NO: 3)
GTDTVYVGQDYPSGLSKRVPARLVAGPMLRERSCHAHVFRAGHMWNWRTSLPSGRWDQPALEKS
RVLTRSVATATDPEITSYPGKSVSTSTQVQEEDWCSRESGWISPGLAPEEPSVVSEITASMVAT
MRVATEEVVLEPQPEQVVTILPEHGRNVPPGLAEQDTASPIEVSVLLPDLAENCPLCGVPSGGL
RLLGKHFAVRHAGVPVTYECRKCAWRSPNSHSISCHVPKCRGRARMPSGDPGIACDLCEARFAT
EVGVAQHKRHVHPVEWNKVRLERRGARGGGIKATKLWSVAEVETLIRLIREHGDSGATYQLIAD
ELGRGKTAEQVRSKKRLLRIDTASNSPDDAEVEEERLESLAVRSSSRSPPSLVATRVREAVARG
ESEGGEEIRAIAALIRDVDQNPCLIETSASDIISKLGRRVDGPKRPRPVVREQTQEKGWVRRLA
RRKREYREAQYLYSRDOARLAAQILDGAASQECALPVDQVYGAFREKWETVGQFHGLGEFRTGA
RADNWEFYSPILAAEVKENLMRMANGTAPGPDRISKKALLDWDPRGEQLARLYTTWLIGGVIPR
VFKECRTKLLPKSSDPVELQDIGGWRPVTIGSMVTRLFSRILTMRLTRACPINPRQRGFLASSS
GCAENLLIFDEIVRRSRRDGGPLAVVFVDFARAFDSISHEHILCVLEEGGLDRHVIGLIRNSYV
DCVTRVGCVEGMTPPIQMKVGVKQGDPMSPLLFNLAMDPLIHKLETAGTGLKWGDLSIATLAFA
DDLVLVSDSEEGMGRSLGILEKFCQLTGLRVQPRKCHGFFMDKGVVNGCGTWEICGSPIHMIPP
GESVRYLGVQVGPGRGVMEPDLIPTVHTWIERISEAPLKPSQRMRVLNSFALPRIIYQADLGKV
TVTKLAQIDGIVRKAVKKWLHLSPSTCNGLLYSRNRDGGLGLLKLERLIPSVRTKRIYRMSRSP
DIWTRRMTSHSVSKSDWEMLWVQAGGERGSAPVMGAVEAAPTDVERSPDYPDWRREENLAWSAL
RVQGVGADQFRGDRTSSSWIAEPASVGFAQRHWLAALALRAGVYPTREFLARGKEKSGAACRRC
PARLESCSHILGQCPFVQANRIARHNKVCVLLATEAERFGWTVIREFRLEDAAGGLKIPDLVCK
KADTVLIVDVTVRYEMDGETLKRAASEKVKHYLPVGQQITDKVGGRCFKVMGFPVGARGKWPAS
NNTVLAELGVPAGRMRTFARLVSRRTLLYSLDILRDFMREPAGRGTRVALIPAATGAAN
Amino acid sequence of Phi29 DNA polymerase
(SEQ ID NO: 4)
PRKMYSCDFETTTKVEDCRVWAYGYMNIEDHSEYKIGNSLDEFMAWVLKVQADLYFHNLKFDGA
FIINWLERNGFKWSADGLPNTYNTIISRMGQWYMIDICLGYKGKRKIHTVIYDSLKKLPFPVKK
IAKDFKLTVLKGDIDYHKERPVGYKITPEEYAYIKNDIQIIAEALLIQFKQGLDRMTAGSDSLK
GFKDIITTKKFKKVFPTLSLGLDKEVRYAYRGGFTWLNDRFKEKEIGEGMVFDVNSLYPAQMYS
RLLPYGEPIVFEGKYVWDEDYPLHIQHIRCEFELKEGYIPTIQIKRSRFYKGNEYLKSSGGEIA
DLWLSNVDLELMKEHYDLYNVEYISGLKFKATTGLFKDFIDKWTYIKTTSEGAIKQLAKLMLNS
LYGKFASNPDVTGKVPYLKENGALGFRLGEEETKDPVYTPMGVFITAWARYTTITAAQACYDRI
IYCDTDSIHLTGTEIPDVIKDIVDPKKLGYWAHESTFKRAKYLROKTYIQDIYMKEVDGKLVEG
SPDDYTDIKFSVKCAGMTDKIKKEVTFENFKVGFSRKMKPKPVQVPGGVVLVDDTFTIK
Amino acid sequence of DNA polymerase delta
(SEQ ID NO: 5)
DGKRRPGPGPGVPPKRARGGLWDDDDAPRPSQFEEDLALMEEMEAEHRLQEQEEEELQSVLEGV
ADGQVPPSAIDPRWLRPTPPALDPQTEPLIFQQLEIDHYVGPAQPVPGGPPPSHGSVPVLRAFG
VTDEGFSVCCHIHGFAPYFYTPAPPGFGPEHMGDLQRELNLAINRDSRGGRELTGPAVLAVELC
SRESMFGYHGHGPSPFLRITVALPRLVAPARRLLEQGIRVAGLGTPSFAPYEANVDFEIRFMVD
TDIVGCNWLELPAGKYALRLKEKATQCQLEADVLWSDVVSHPPEGPWQRIAPLRVLSFDIECAG
RKGIFPEPERDPVIQICSLGLRWGEPEPFLRLALTLRPCAPILGAKVQSYEKEEDLLQAWSTFI
RIMDPDVITGYNIQNFDLPYLISRAQTLKVQTFPFLGRVAGLCSNIRDSSFQSKQTGRRDTKVV
KDAYLPLRLLERLMVLVNAVEMARVTGVPLSYLLSRGQQVKVVSQLLRQAMHEGLLMPVVKSEG
GEDYTGATVIEPLKGYYDVPIATLDFSSLYPSIMMAHNLCYTTLLRPGTAQKLGLTEDQFIRTP
TGDEFVKTSVRKGLLPQILENLLSARKRAKAELAKETDPLRRQVLDGRQLALKVSANSVYGFTG
AQVGKLPCLEISQSVTGFGRQMIEKTKQLVESKYTVENGYSTSAKVVYGDTDSVMCRFGVSSVA
EAMALGGEAADWVSGHFPSPIRLEFEKVYFPYLLISKKRYAGLLFSSRPDAHDRMDCKGLEAVR
RDNCPLVANLVTASLRRLLIDRDPEGAVAHAQDVISDLLCNRIDISQLVITKELTRAASDYAGK
QAHVELAERMRKRDPGSAPSLGDRVPYVIISAAKGVAAYMKSEDPLFVLEHSLPIDTQYYLEQQ
LAKPLLRIFEPILGEGRAEAVLLRGDHTRCKTVLTGKVGGLLAFAKRRNCCIGCRTVLSHQGAV
CEFCQPRESELYQKEVSHLNALEERFSRLWTQCQRCQGSLHEDVICTSRDCPIFYMRKKVRKDL
EDQEQLLRRFGPPGPEAW
Amino acid sequence of T4 DNA polymerase
(SEQ ID NO: 6)
PSMKDARDWMKRMEDIGLEALGMNDFKLAYISDTYGSEIVYDRKFVRVANCDIEVTGDKFPDPM
KAEYEIDAITHYDSIDDRFYVFDLLNSMYGSVSKWDAKLAAKLDCEGGDEVPQEILDRVIYMPF
DNERDMLMEYINLWEQKRPAIFTGWNIEGFDVPYIMNRVKMILGERSMKRFSPIGRVKSKLIQN
MYGSKEIYSIDGVSILDYLDLYKKFAFTNLPSFSLESVAQHETKKGKLPYDGPINKLRETNHQR
YISYNIIDVESVQAIDKIRGFIDLVLSMSYYAKMPFSGVMSPIKTWDAIIFNSLKGEHKVIPQQ
GSHVKQSFPGAFVFEPKPIARRYIMSFDLTSLYPSIIRQVNISPETIRGQFKVHPTHEYIAGTA
PKPSDEYSCSPNGWMYDKHQEGIIPKEIAKVFFQRKDWKKKMFAEEMNAEAIKKIIMKGAGSCS
TKPEVERYVKFSDDFLNELSNYTESVLNSLIEECEKAATLANTNOLNRKILINSLYGALGNIHF
RYYDLRNATAITIFGQVGIQWIARKINEYLNKVCGTNDEDFIAAGDTDSVYVCVDKVIEKVGLD
RFKEQNDLVEFMNQFGKKKMEPMIDVAYRELCDYMNNREHLMHMDREAISCPPLGSKGVGGFWK
AKKRYALNVYDMEDKRFAEPHLKIMGMETQQSSTPKAVQEALEESIRRILQEGEESVQEYYKNF
EKEYROLDYKVIAEVKTANDIAKYDDKGWPGFKCPFHIRGVLTYRRAVSGLGVAPILDGNKVMV
LPLREGNPFGDKCIAWPSGTELPKEIRSDVLSWIDHSTLFQKSFVKPLAGMCESAGMDYEEKAS
LDFLFG
Amino acid sequence of T4 DNA ligase
(SEQ ID NO: 7)
ILKILNEIASIGSTKQKQAILEKNKDNELLKRVYRLTYSRGLQYYIKKWPKPGIATQSFGMLTL
TDMLDFIEFTLATRKLTGNAAIEELTGYITDGKKDDVEVLRRVMMRDLECGASVSIANKVWPGL
IPEQPQMLASSYDEKGINKNIKFPAFAQLKADGARCFAEVRGDELDDVRLLSRAGNEYLGLDLL
KEELIKMTAEARQIHPEGVLIDGELVYHEQVKKEPEGLDFLFDAYPENSKAKEFAEVAESRTAS
NGIANKSLKGTISEKEAQCMKFQVWDYVPLVEIYSLPAFRLKYDVRFSKLEQMTSGYDKVILIE
NQVVNNLDEAKVIYKKYIDQGLEGIILKNIDGLWENARSKNLYKFKEVIDVDLKIVGIYPHRKD
PTKAGGFILESECGKIKVNAGSGLKDKAGVKSHELDRTRIMENQNYYIGKILECECNGWLKSDG
RTDYVKLFLPIAIRLREDKTKANTFEDVFGDFHEVTGL
Amino acid sequence of MEPC2
(SEQ ID NO: 8)
ASNFTQFVLVDNGGTGDVTVAPSNFANGVAEWISSNSRSQAYKVTCSVRQSSAQKRKYTIKVEV
PKVATQTVGGVELPVAAWRSYLNMELTIPIFATNSDCELIVKAMQGLLKDGNPIPSAIAANSGI
Y
Amino acid sequence of Rep protein
(SEQ ID NO: 9)
PGFYEIVIKVPSDLDGHLPGISDSFVNWVAEKEWELPPDSDMDLNLIEQAPLTVAEKLQRDFLT
EWRRVSKAPEALFFVQFEKGESYFHMHVLVETTGVKSMVLGRFLSQIREKLIQRIYRGIEPTLP
NWFAVTKTRNGAGGGNKVVDECYIPNFLLPKTQPELQWAWTNMEQYLSACLNLTERKRLVAQHL
THVS
Amino acid sequence of T4 Gene 32 Protein
(SEQ ID NO: 10)
MFKRKSTAELAAQMAKLNGNKGFSSEDKGEWKLKLDNAGNGQAVIRFLPSKNDEQAPFAILVNH
GFKKNGKWYIETCSSTHGDYDSCPVCQYISKNDLYNTDNKEYSLVKRKTSYWANILVVKDPAAP
ENEGKVFKYRFGKKIWDKINAMIAVDVEMGETPVDVTCPWEGANFVLKVKQVSGFSNYDESKFL
NQSAIPNIDDESFQKELFEQMVDLSEMTSKDKFKSFEELNTKFGQVMGTAVMGGAAATAAKKAD
KVADDLDAFNVDDFNTKTEDDFMSSSSGSSSSADDTDLDDLLNDL
Amino acid sequence of FUBP
(SEQ ID NO: 11)
MADYSTVPPPSSGSAGGGGGGGGGGGVNDAFKDALQRARQIAAKIGGDAGTSLNSNDYGYGGQK
RPLEDGDQPDAKKVAPQNDSFGTQLPPMHQQQSRSVMTEEYKVPDGMVGFIIGRGGEQISRIQQ
ESGCKIQIAPDSGGLPERSCMLTGTPESVQSAKRLLDQIVEKGRPAPGFHHGDGPGNAVQEIMI
PASKAGLVIGKGGETIKQLQERAGVKMVMIQDGPQNTGADKPLRITGDPYKVQQAKEMVLELIR
DOGGFREVRNEYGSRIGGNEGIDVPIPRFAVGIVIGRNGEMIKKIQNDAGVRIQFKPDDGTTPE
RIAQITGPPDRCQHAAEIITDLLRSVQAGNPGGPGPGGRGRGRGQGNWNMGPPGGLQEFNFIVP
TGKTGLIIGKGGETIKSISQQSGARIELQRNPPPNADPNMKLFTIRGTPQQIDYARQLIEEKIG
GPVNPLGPPVPHGPHGVPGPHGPPGPPGPGTPMGPYNPAPYNPGPPGPAPHGPPAPYAPQGWGN
AYPHWQQQAPPDPAKAGTDPNSAAWAAYYAHYYQQQAQPPPAAPAGAPTTTQTNGQGDQQNPAP
AGQVDYTKAWEEYYKKMGQAVPAPTGAPPGGQPDYSAAWAEYYRQQAAYYAQTSPQGMPQHPPA
PQGQ
Nuclear localization sequences (SEQ ID NOS: 12-14)
(SEQ ID NO: 12)
MKRTADGSEFESPKKKRKV
(SEQ ID NO: 13)
SGGSKRTADGSEFEPKKKRKV
(SEQ ID NO: 14)
PKKKRKV
Linker sequences (SEQ ID NOS: 15-16)
(SEQ ID NO: 15)
SGGSSGGSSGSETPGTSESATPESSGGSSGGS
(SEQ ID NO: 16)
SGGSSGGSSGSETPGTSESATPESSG
Amino acid sequence of REP_Y156F(1-197)-Cas9 P2A EGFP
(SEQ ID NO: 17)
MKRTADGSEFESPKKKRKVPGFYEIVIKVPSDLDGHLPGISDSFVNWVAEKEWELPPDSDMDLN
LIEQAPLTVAEKLQRDFLTEWRRVSKAPEALFFVQFEKGESYFHMHVLVETTGVKSMVLGRFLS
QIREKLIQRIYRGIEPTLPNWFAVTKTRNGAGGGNKVVDECYIPNFLLPKTQPELQWAWTNMEQ
YLSACLNLTERKRLVAQHLTHVSSGGSSGGSSGSETPGTSESATPESSGGSSGGSDKKYSIGLD
IGTNSVGWAVITDEYKVPSKKFKVLGNTDRHSIKKNLIGALLFDSGETAEATRLKRTARRRYTR
RKNRICYLQEIFSNEMAKVDDSFFHRLEESFLVEEDKKHERHPIFGNIVDEVAYHEKYPTIYHL
RKKLVDSTDKADLRLIYLALAHMIKFRGHFLIEGDLNPDNSDVDKLNIQLVQTYNQLFEENPIN
ASGVDAKAILSARLSKSRRLENLIAQLPGEKKNGLFGNLIALSLGLTPNFKSNFDLAEDAKLQL
SKDTYDDDLDNLLAQIGDQYADLFLAAKNLSDAILLSDILRVNTEITKAPLSASMIKRYDEHHQ
DLTLLKALVRQQLPEKYKEIFFDQSKNGYAGYIDGGASQEEFYKFIKPILEKMDGTEELLVKLN
REDLLRKORTFDNGSIPHQIHLGELHAILRRQEDFYPFLKDNREKIEKILTFRIPYYVGPLARG
NSRFAWMTRKSEETITPWNFEEVVDKGASAQSFIERMTNFDKNLPNEKVLPKHSLLYEYFTVYN
ELTKVKYVTEGMRKPAFLSGEQKKAIVDLLFKTNRKVTVKQLKEDYFKKIECFDSVEISGVEDR
FNASLGTYHDLLKIIKDKDFLDNEENEDILEDIVLTLTLFEDREMIEERLKTYAHLFDDKVMKQ
LKRRRYTGWGRLSRKLINGIRDKQSGKTILDFLKSDGFANRNFMQLIHDDSLTFKEDIQKAQVS
GQGDSLHEHIANLAGSPAIKKGILQTVKVVDELVKVMGRHKPENIVIEMARENQTSDYDVDHIV
PQSFLKDDSIDNKVLTRSDKNRGKSDNVPSEEVVKKMKNYWRQLLNAKLITQRKFDNLTKAERG
GLSELDKAGFIKRQLVETRQITKHVAQILDSRMNTKYDENDKLIREVKVITLKSKLVSDFRKDF
QFYKVREINNYHHAHDAYLNAVVGTALIKKYPKLESEFVYGDYKVYDVRKMIAKSEQEIGKATA
KYFFYSNIMNFFKTEITLANGEIRKRPLIETNGETGEIVWDKGRDFATVRKVLSMPQVNIVKKT
EVQTGGFSKESILPKRNSDKLIARKKDWDPKKYGGFDSPTVAYSVLVVAKVEKGKSKKLKSVKE
LLGITIMERSSFEKNPIDFLEAKGYKEVKKDLIIKLPKYSLFELENGRKRMLASAGELQKGNEL
ALPSKYVNFLYLASHYEKLKGSPEDNEQKQLFVEQHKHYLDEIIEQISEFSKRVILADANLDKV
LSAYNKHRDKPIREQAENIIHLFTLTNLGAPAAFKYFDTTIDRKRYTSTKEVLDATLIHQSITG
LYETRIDLSQLGGDSGGSKRTADGSEFEPKKKRKVGSGATNFSLLKQAGDVEENPGPMVSKGEE
LFTGVVPILVELDGDVNGHKFSVSGEGEGDATYGKLTLKFICTTGKLPVPWPTLVTTLTYGVQC
FSRYPDHMKQHDFFKSAMPEGYVQERTIFFKDDGNYKTRAEVKFEGDTLVNRIELKGIDFKEDG
NILGHKLEYNYNSHNVYIMADKQKNGIKVNFKIRHNIEDGSVQLADHYQQNTPIGDGPVLLPDN
HYLSTQSALSKDPNEKRDHMVLLEFVTAAGITLGMDELYKSGGSPKKKRKV
Amino acid sequence of Cas9-MMLV RT
(SEQ ID NO: 18)
PKKKRKVDKKYSIGLDIGTNSVGWAVITDEYKVPSKKFKVLGNTDRHSIKKNLIGALLFDSGET
AEATRLKRTARRRYTRRKNRICYLQEIFSNEMAKVDDSFFHRLEESFLVEEDKKHERHPIFGNI
VDEVAYHEKYPTIYHLRKKLVDSTDKADLRLIYLALAHMIKFRGHFLIEGDLNPDNSDVDKLFI
QLVQTYNQLFEENPINASGVDAKAILSARLSKSRRLENLIAQLPGEKKNGLFGNLIALSLGLTP
NFKSNFDLAEDAKLQLSKDTYDDDLDNLLAQIGDQYADLFLAAKNLSDAILLSDILRVNTEITK
APLSASMIKRYDEHHQDLTLLKALVRQQLPEKYKEIFFDQSKNGYAGYIDGGASQEEFYKFIKP
ILEKMDGTEELLVKLNREDLLRKQRTFDNGSIPHQIHLGELHAILRRQEDFYPFLKDNREKIEK
ILTFRIPYYVGPLARGNSRFAWMTRKSEETITPWNFEEVVDKGASAQSFIERMTNFDKNLPNEK
VLPKHSLLYEYFTVYNELTKVKYVTEGMRKPAFLSGEQKKAIVDLLFKTNRKVTVKQLKEDYFK
KIECFDSVEISGVEDRFNASLGTYHDLLKIIKDKDFLDNEENEDILEDIVLTLTLFEDREMIEE
RLKTYAHLFDDKVMKQLKRRRYTGWGRLSRKLINGIRDKQSGKTILDFLKSDGFANRNFMQLIH
DDSLTFKEDIQKAQVSGQGDSLHEHIANLAGSPAIKKGILQTVKVVDELVKVMGRHKPENIVIE
MARENQTTQKGQKNSRERMKRIEEGIKELGSQILKEHPVENTQLQNEKLYLYYLQNGRDMYVDQ
ELDINRLSDYDVDHIVPQSFLKDDSIDNKVLTRSDKNRGKSDNVPSEEVVKKMKNYWRQLLNAK
LITQRKFDNLTKAERGGLSELDKAGFIKRQLVETRQITKHVAQILDSRMNTKYDENDKLIREVK
VITLKSKLVSDFRKDFQFYKVREINNYHHAHDAYLNAVVGTALIKKYPKLESEFVYGDYKVYDV
RKMIAKSEQEIGKATAKYFFYSNIMNFFKTEITLANGEIRKRPLIETNGETGEIVWDKGRDFAT
VRKVLSMPQVNIVKKTEVQTGGFSKESILPKRNSDKLIARKKDWDPKKYGGFDSPTVAYSVLVV
AKVEKGKSKKLKSVKELLGITIMERSSFEKNPIDFLEAKGYKEVKKDLIIKLPKYSLFELENGR
KRMLASAGELQKGNELALPSKYVNFLYLASHYEKLKGSPEDNEQKQLFVEQHKHYLDEIIEQIS
EFSKRVILADANLDKVLSAYNKHRDKPIREQAENIIHLFTLTNLGAPAAFKYFDTTIDRKRYTS
TKEVLDATLIHQSITGLYETRIDLSQLGGDSGGSSGGSSGSETPGTSESATPESSGGSSGGSST
LNIEDEYRLHETSKEPDVSLGSTWLSDFPQAWAETGGMGLAVRQAPLIIPLKATSTPVSIKQYV
PNPYNLLSGLPPSHQWYTVLDLKDAFFCLRLHPTSQPLFAFEWRDPEMGISGQLTWTRLPQGFK
NSPTLFNEALHRDLADFRIQHPDLILLQYVDDLLLAATSELDCQQGTRALLQTLGNLGYRASAK
KAQICQKQVKYLGYLLKEGQRWLTEARKETVMGQPTPKTPRQLREFLGKAGFCRLFIPGFAEMA
APLYPLTKPGTLFNWGPDQQKAYQEIKQALLTAPALGLPDLTKPFELFVDEKQGYAKGVLTQKL
GPWRRPVAYLSKKLDPVAAGWPPCLRMVAAIAVLTKDAGKLTMGQPLVILAPHAVEALVKQPPD
RWLSNARMTHYQALLLDTDRVQFGPVVALNPATLLPLPEEGLQHNCLDILAEAHGTRPDLTDQP
LPDADHTWYTDGSSLLQEGQRKAGAAVTTETEVIWAKALPAGTSAQRAELIALTQALKMAEGKK
LNVYTDSRYAFATAHIHGEIYRRRGWLTSEGKEIKNKDEILALLKALFLPKRLSIIHCPGHQKG
HSAEARGNRMADQAARKAAITETPDTSTLLIENSSPSGGSKRTADGSEFEPKKKRKV
Amino acid sequence of MCP2-RT
(SEQ ID NO: 19)
ASNFTQFVLVDNGGTGDVTVAPSNFANGVAEWISSNSRSQAYKVTCSVRQSSAQKRKYTIKVEV
PKVATQTVGGVELPVAAWRSYLNMELTIPIFATNSDCELIVKAMQGLLKDGNPIPSAIAANSGI
YPKKKRKVTLNIEDEYRLHETSKEPDVSLGSTWLSDFPQAWAETGGMGLAVRQAPLIIPLKATS
TPVSIKQYVPNPYNLLSGLPPSHQWYTVLDLKDAFFCLRLHPTSQPLFAFEWRDPEMGISGQLT
WTRLPQGFKNSPTLFNEALHRDLADFRIQHPDLILLQYVDDLLLAATSELDCQQGTRALLQTLG
NLGYRASAKKAQICQKQVKYLGYLLKEGQRWLTEARKETVMGQPTPKTPRQLREFLGKAGFCRL
FIPGFAEMAAPLYPLTKPGTLFNWGPDQQKAYQEIKQALLTAPALGLPDLTKPFELFVDEKQGY
AKGVLTQKLGPWRRPVAYLSKKLDPVAAGWPPCLRMVAAIAVLTKDAGKLTMGQPLVILAPHAV
EALVKQPPDRWLSNARMTHYQALLLDTDRVQFGPVVALNPATLLPLPEEGLQHNCLDILAEAHG
TRPDLTDQPLPDADHTWYTDGSSLLQEGQRKAGAAVTTETEVIWAKALPAGTSAQRAELIALTQ
ALKMAEGKKLNVYTDSRYAFATAHIHGEIYRRRGWLTSEGKEIKNKDEILALLKALFLPKRLSI
IHCPGHQKGHSAEARGNRMADQAARKAAITETPDTSTLLIENSSPSGGSKRTADGSEFEPKKKR
KV
Amino acid sequence of Cas9-Phi29
(SEQ ID NO: 20)
PKKKRKVDKKYSIGLDIGTNSVGWAVITDEYKVPSKKFKVLGNTDRHSIKKNLIGALLFDSGET
AEATRLKRTARRRYTRRKNRICYLQEIFSNEMAKVDDSFFHRLEESFLVEEDKKHERHPIFGNI
VDEVAYHEKYPTIYHLRKKLVDSTDKADLRLIYLALAHMIKFRGHFLIEGDLNPDNSDVDKLFI
QLVQTYNQLFEENPINASGVDAKAILSARLSKSRRLENLIAQLPGEKKNGLFGNLIALSLGLTP
NFKSNFDLAEDAKLQLSKDTYDDDLDNLLAQIGDQYADLFLAAKNLSDAILLSDILRVNTEITK
APLSASMIKRYDEHHQDLTLLKALVRQQLPEKYKEIFFDQSKNGYAGYIDGGASQEEFYKFIKP
ILEKMDGTEELLVKLNREDLLRKQRTFDNGSIPHQIHLGELHAILRRQEDFYPFLKDNREKIEK
ILTFRIPYYVGPLARGNSRFAWMTRKSEETITPWNFEEVVDKGASAQSFIERMTNFDKNLPNEK
VLPKHSLLYEYFTVYNELTKVKYVTEGMRKPAFLSGEQKKAIVDLLFKTNRKVTVKQLKEDYFK
KIECFDSVEISGVEDRFNASLGTYHDLLKIIKDKDFLDNEENEDILEDIVLTLTLFEDREMIEE
RLKTYAHLFDDKVMKQLKRRRYTGWGRLSRKLINGIRDKQSGKTILDFLKSDGFANRNFMQLIH
DDSLTFKEDIQKAQVSGQGDSLHEHIANLAGSPAIKKGILQTVKVVDELVKVMGRHKPENIVIE
MARENQTTQKGQKNSRERMKRIEEGIKELGSQILKEHPVENTQLQNEKLYLYYLQNGRDMYVDQ
ELDINRLSDYDVDHIVPQSFLKDDSIDNKVLTRSDKNRGKSDNVPSEEVVKKMKNYWRQLLNAK
LITORKFDNLTKAERGGLSELDKAGFIKRQLVETRQITKHVAQILDSRMNTKYDENDKLIREVK
VITLKSKLVSDFRKDFQFYKVREINNYHHAHDAYLNAVVGTALIKKYPKLESEFVYGDYKVYDV
RKMIAKSEQEIGKATAKYFFYSNIMNFFKTEITLANGEIRKRPLIETNGETGEIVWDKGRDFAT
VRKVLSMPQVNIVKKTEVQTGGFSKESILPKRNSDKLIARKKDWDPKKYGGFDSPTVAYSVLVV
AKVEKGKSKKLKSVKELLGITIMERSSFEKNPIDFLEAKGYKEVKKDLIIKLPKYSLFELENGR
KRMLASAGELQKGNELALPSKYVNFLYLASHYEKLKGSPEDNEQKQLFVEQHKHYLDEIIEQIS
EFSKRVILADANLDKVLSAYNKHRDKPIREQAENIIHLFTLTNLGAPAAFKYFDTTIDRKRYTS
TKEVLDATLIHQSITGLYETRIDLSQLGGDSGGSSGGSSGSETPGTSESATPESSGGSSGGSSP
RKMYSCDFETTTKVEDCRVWAYGYMNIEDHSEYKIGNSLDEFMAWVLKVQADLYFHNLKFDGAF
IINWLERNGFKWSADGLPNTYNTIISRMGQWYMIDICLGYKGKRKIHTVIYDSLKKLPFPVKKI
AKDFKLTVLKGDIDYHKERPVGYKITPEEYAYIKNDIQIIAEALLIQFKOGLDRMTAGSDSLKG
FKDIITTKKFKKVFPTLSLGLDKEVRYAYRGGFTWLNDRFKEKEIGEGMVFDVNSLYPAQMYSR
LLPYGEPIVFEGKYVWDEDYPLHIQHIRCEFELKEGYIPTIQIKRSRFYKGNEYLKSSGGEIAD
LWLSNVDLELMKEHYDLYNVEYISGLKFKATTGLFKDFIDKWTYIKTTSEGAIKQLAKLMLNSL
YGKFASNPDVTGKVPYLKENGALGFRLGEEETKDPVYTPMGVFITAWARYTTITAAQACYDRII
YCDTDSIHLTGTEIPDVIKDIVDPKKLGYWAHESTFKRAKYLROKTYIQDIYMKEVDGKLVEGS
PDDYTDIKFSVKCAGMTDKIKKEVTFENFKVGFSRKMKPKPVQVPGGVVLVDDTFTIKPKKKRK
V
Amino acid sequence of Cas9-PolD
(SEQ ID NO: 21)
PKKKRKVDKKYSIGLDIGTNSVGWAVITDEYKVPSKKFKVLGNTDRHSIKKNLIGALLFDSGET
AEATRLKRTARRRYTRRKNRICYLQEIFSNEMAKVDDSFFHRLEESFLVEEDKKHERHPIFGNI
VDEVAYHEKYPTIYHLRKKLVDSTDKADLRLIYLALAHMIKFRGHFLIEGDLNPDNSDVDKLFI
QLVQTYNQLFEENPINASGVDAKAILSARLSKSRRLENLIAQLPGEKKNGLFGNLIALSLGLTP
NFKSNFDLAEDAKLQLSKDTYDDDLDNLLAQIGDQYADLFLAAKNLSDAILLSDILRVNTEITK
APLSASMIKRYDEHHQDLTLLKALVRQQLPEKYKEIFFDQSKNGYAGYIDGGASQEEFYKFIKP
ILEKMDGTEELLVKLNREDLLRKORTFDNGSIPHQIHLGELHAILRRQEDFYPFLKDNREKIEK
ILTFRIPYYVGPLARGNSRFAWMTRKSEETITPWNFEEVVDKGASAQSFIERMTNFDKNLPNEK
VLPKHSLLYEYFTVYNELTKVKYVTEGMRKPAFLSGEQKKAIVDLLFKTNRKVTVKQLKEDYFK
KIECFDSVEISGVEDRFNASLGTYHDLLKIIKDKDFLDNEENEDILEDIVLTLTLFEDREMIEE
RLKTYAHLFDDKVMKQLKRRRYTGWGRLSRKLINGIRDKQSGKTILDFLKSDGFANRNFMQLIH
DDSLTFKEDIQKAQVSGQGDSLHEHIANLAGSPAIKKGILQTVKVVDELVKVMGRHKPENIVIE
MARENQTTQKGQKNSRERMKRIEEGIKELGSQILKEHPVENTQLQNEKLYLYYLQNGRDMYVDQ
ELDINRLSDYDVDHIVPQSFLKDDSIDNKVLTRSDKNRGKSDNVPSEEVVKKMKNYWRQLLNAK
LITQRKFDNLTKAERGGLSELDKAGFIKRQLVETRQITKHVAQILDSRMNTKYDENDKLIREVK
VITLKSKLVSDFRKDFQFYKVREINNYHHAHDAYLNAVVGTALIKKYPKLESEFVYGDYKVYDV
RKMIAKSEQEIGKATAKYFFYSNIMNFFKTEITLANGEIRKRPLIETNGETGEIVWDKGRDFAT
VRKVLSMPQVNIVKKTEVQTGGFSKESILPKRNSDKLIARKKDWDPKKYGGFDSPTVAYSVLVV
AKVEKGKSKKLKSVKELLGITIMERSSFEKNPIDFLEAKGYKEVKKDLIIKLPKYSLFELENGR
KRMLASAGELQKGNELALPSKYVNFLYLASHYEKLKGSPEDNEQKOLFVEQHKHYLDEIIEQIS
EFSKRVILADANLDKVLSAYNKHRDKPIREQAENIIHLFTLTNLGAPAAFKYFDTTIDRKRYTS
TKEVLDATLIHQSITGLYETRIDLSQLGGDSGGSSGGSSGSETPGTSESATPESSGDGKRRPGP
GPGVPPKRARGGLWDDDDAPRPSQFEEDLALMEEMEAEHRLQEQEEEELQSVLEGVADGQVPPS
AIDPRWLRPTPPALDPQTEPLIFQQLEIDHYVGPAQPVPGGPPPSHGSVPVLRAFGVTDEGFSV
CCHIHGFAPYFYTPAPPGFGPEHMGDLQRELNLAINRDSRGGRELTGPAVLAVELCSRESMFGY
HGHGPSPFLRITVALPRLVAPARRLLEQGIRVAGLGTPSFAPYEANVDFEIRFMVDTDIVGCNW
LELPAGKYALRLKEKATQCQLEADVLWSDVVSHPPEGPWQRIAPLRVLSFDIECAGRKGIFPEP
ERDPVIQICSLGLRWGEPEPFLRLALTLRPCAPILGAKVQSYEKEEDLLQAWSTFIRIMDPDVI
TGYNIQNFDLPYLISRAQTLKVQTFPFLGRVAGLCSNIRDSSFQSKQTGRRDTKVVSMVGRVQM
DMLQVLLREYKLRSYTLNAVSFHFLGEQKEDVQHSIITDLQNGNDQTRRRLAVYCLKDAYLPLR
LLERLMVLVNAVEMARVTGVPLSYLLSRGQQVKVVSQLLRQAMHEGLLMPVVKSEGGEDYTGAT
VIEPLKGYYDVPIATLDFSSLYPSIMMAHNLCYTTLLRPGTAQKLGLTEDQFIRTPTGDEFVKT
SVRKGLLPQILENLLSARKRAKAELAKETDPLRRQVLDGRQLALKVSANSVYGFTGAQVGKLPC
LEISQSVTGFGRQMIEKTKQLVESKYTVENGYSTSAKVVYGDTDSVMCRFGVSSVAEAMALGGE
AADWVSGHFPSPIRLEFEKVYFPYLLISKKRYAGLLFSSRPDAHDRMDCKGLEAVRRDNCPLVA
NLVTASLRRLLIDRDPEGAVAHAQDVISDLLCNRIDISQLVITKELTRAASDYAGKQAHVELAE
RMRKRDPGSAPSLGDRVPYVIISAAKGVAAYMKSEDPLFVLEHSLPIDTQYYLEQQLAKPLLRI
FEPILGEGRAEAVLLRGDHTRCKTVLTGKVGGLLAFAKRRNCCIGCRTVLSHQGAVCEFCQPRE
SELYQKEVSHLNALEERFSRLWTQCQRCQGSLHEDVICTSRDCPIFYMRKKVRKDLEDQEQLLR
RFGPPGPEAWSGGSSGGSSGSETPGTSESATPESSGGSSGGSSPKKKRKV
Amino acid sequence of Cas9-R2 RT
(SEQ ID NO: 22)
PKKKRKVDKKYSIGLDIGTNSVGWAVITDEYKVPSKKFKVLGNTDRHSIKKNLIGALLFDSGET
AEATRLKRTARRRYTRRKNRICYLQEIFSNEMAKVDDSFFHRLEESFLVEEDKKHERHPIFGNI
VDEVAYHEKYPTIYHLRKKLVDSTDKADLRLIYLALAHMIKFRGHFLIEGDLNPDNSDVDKLFI
QLVQTYNQLFEENPINASGVDAKAILSARLSKSRRLENLIAQLPGEKKNGLFGNLIALSLGLTP
NFKSNFDLAEDAKLQLSKDTYDDDLDNLLAQIGDQYADLFLAAKNLSDAILLSDILRVNTEITK
APLSASMIKRYDEHHQDLTLLKALVROQLPEKYKEIFFDQSKNGYAGYIDGGASQEEFYKFIKP
ILEKMDGTEELLVKLNREDLLRKORTFDNGSIPHQIHLGELHAILRRQEDFYPFLKDNREKIEK
ILTFRIPYYVGPLARGNSRFAWMTRKSEETITPWNFEEVVDKGASAQSFIERMTNFDKNLPNEK
VLPKHSLLYEYFTVYNELTKVKYVTEGMRKPAFLSGEQKKAIVDLLFKTNRKVTVKQLKEDYFK
KIECFDSVEISGVEDRFNASLGTYHDLLKIIKDKDFLDNEENEDILEDIVLTLTLFEDREMIEE
RLKTYAHLFDDKVMKQLKRRRYTGWGRLSRKLINGIRDKQSGKTILDFLKSDGFANRNFMQLIH
DDSLTFKEDIQKAQVSGQGDSLHEHIANLAGSPAIKKGILQTVKVVDELVKVMGRHKPENIVIE
MARENQTTQKGQKNSRERMKRIEEGIKELGSQILKEHPVENTQLQNEKLYLYYLQNGRDMYVDQ
ELDINRLSDYDVDHIVPQSFLKDDSIDNKVLTRSDKNRGKSDNVPSEEVVKKMKNYWRQLLNAK
LITQRKFDNLTKAERGGLSELDKAGFIKRQLVETRQITKHVAQILDSRMNTKYDENDKLIREVK
VITLKSKLVSDFRKDFQFYKVREINNYHHAHDAYLNAVVGTALIKKYPKLESEFVYGDYKVYDV
RKMIAKSEQEIGKATAKYFFYSNIMNFFKTEITLANGEIRKRPLIETNGETGEIVWDKGRDFAT
VRKVLSMPQVNIVKKTEVQTGGFSKESILPKRNSDKLIARKKDWDPKKYGGFDSPTVAYSVLVV
AKVEKGKSKKLKSVKELLGITIMERSSFEKNPIDFLEAKGYKEVKKDLIIKLPKYSLFELENGR
KRMLASALADANLDKVLSAYNKHRDKPIREQAENIIHLFTLTNLGAPAAFKYFDTTIDRKRYTS
TKEVLDATLIHQSITGLYETRIDLSQLGGDSGGSSGGSSGSETPGTSESATPESSGGSSGGSSG
TDTVYVGQDYPSGLSKRVPARLVAGPMLRERSCHAHVFRAGHMWNWRTSLPSGRWDQPALEKSR
VLTRSVATATDPEITSYPGKSVSTSTQVQEEDWCSRESGWISPGLAPEEPSVVSEITASMVATM
RVATEEVVLEPQPEQVVTILPEHGRNVPPGLAEQDTASPIEVSVLLPDLAENCPLCGVPSGGLR
LLGKHFAVRHAGVPVTYECRKCAWRSPNSHSISCHVPKCRGRARMPSGDPGIACDLCEARFATE
VGVAQHKRHVHPVEWNKVRLERRGARGGGIKATKLWSVAEVETLIRLIREHGDSGATYQLIADE
LGRGKTAEQVRSKKRLLRIDTASNSPDDAEVEEERLESLAVRSSSRSPPSLVATRVREAVARGE
SEGGEEIRAIAALIRDVDONPCLIETSASDIISKLGRRVDGPKRPRPVVREQTQEKGWVRRLAR
RKREYREAQYLYSRDOARLAAQILDGAASQECALPVDQVYGAFREKWETVGQFHGLGEFRTGAR
ADNWEFYSPILAAEVKENLMRMANGTAPGPDRISKKALLDWDPRGEQLARLYTTWLIGGVIPRV
FKECRTKLLPKSSDPVELQDIGGWRPVTIGSMVTRLFSRILTMRLTRACPINPRQRGFLASSSG
CAENLLIFDEIVRRSRRDGGPLAVVFVDFARAFDSISHEHILCVLEEGGLDRHVIGLIRNSYVD
CVTRVGCVEGMTPPIQMKVGVKQGDPMSPLLFNLAMDPLIHKLETAGTGLKWGDLSIATLAFAD
DLVLVSDSEEGMGRSLGILEKFCQLTGLRVQPRKCHGFFMDKGVVNGCGTWEICGSPIHMIPPG
ESVRYLGVQVGPGRGVMEPDLIPTVHTWIERISEAPLKPSQRMRVLNSFALPRIIYQADLGKVT
VTKLAQIDGIVRKAVKKWLHLSPSTCNGLLYSRNRDGGLGLLKLERLIPSVRTKRIYRMSRSPD
IWTRRMTSHSVSKSDWEMLWVQAGGERGSAPVMGAVEAAPTDVERSPDYPDWRREENLAWSALR
VQGVGADQFRGDRTSSSWIAEPASVGFAQRHWLAALALRAGVYPTREFLARGKEKSGAACRRCP
ARLESCSHILGQCPFVQANRIARHNKVCVLLATEAERFGWTVIREFRLEDAAGGLKIPDLVCKK
ADTVLIVDVTVRYEMDGETLKRAASEKVKHYLPVGQQITDKVGGRCFKVMGFPVGARGKWPASN
NTVLAELGVPAGRMRTFARLVSRRTLLYSLDILRDFMREPAGRGTRVALIPAATGAANPKKKRK
V
Amino acid sequence of Cas9-T4 DNA ligase
(SEQ ID NO: 23)
DKKYSIGLDIGTNSVGWAVITDEYKVPSKKFKVLGNTDRHSIKKNLIGALLFDSGETAEATRLK
RTARRRYTRRKNRICYLQEIFSNEMAKVDDSFFHRLEESFLVEEDKKHERHPIFGNIVDEVAYH
EKYPTIYHLRKKLVDSTDKADLRLIYLALAHMIKFRGHFLIEGDLNPDNSDVDKLFIQLVQTYN
QLFEENPINASGVDAKAILSARLSKSRRLENLIAQLPGEKKNGLFGNLIALSLGLTPNFKSNFD
LAEDAKLQLSKDTYDDDLDNLLAQIGDQYADLFLAAKNLSDAILLSDILRVNTEITKAPLSASM
IKRYDEHHQDLTLLKALVRQQLPEKYKEIFFDQSKNGYAGYIDGGASQEEFYKFIKPILEKMDG
TEELLVKLNREDLLRKORTFDNGSIPHQIHLGELHAILRRQEDFYPFLKDNREKIEKILTFRIP
YYVGPLARGNSRFAWMTRKSEETITPWNFEEVVDKGASAQSFIERMTNFDKNLPNEKVLPKHSL
LYEYFTVYNELTKVKYVTEGMRKPAFLSGEQKKAIVDLLFKTNRKVTVKQLKEDYFKKIECFDS
VEISGVEDRFNASLGTYHDLLKIIKDKDFLDNEENEDILEDIVLTLTLFEDREMIEERLKTYAH
LFDDKVMKQLKRRRYTGWGRLSRKLINGIRDKQSGKTILDFLKSDGFANRNFMQLIHDDSLTFK
EDIQKAQVSGQGDSLHEHIANLAGSPAIKKGILQTVKVVDELVKVMGRHKPENIVIEMARENQT
TQKGQKNSRERMKRIEEGIKELGSQILKEHPVENTQLQNEKLYLYYLQNGRDMYVDQELDINRL
SDYDVDHIVPQSFLKDDSIDNKVLTRSDKNRGKSDNVPSEEVVKKMKNYWRQLLNAKLITQRKF
DNLTKAERGGLSELDKAGFIKRQLVETRQITKHVAQILDSRMNTKYDENDKLIREVKVITLKSK
LVSDFRKDFQFYKVREINNYHHAHDAYLNAVVGTALIKKYPKLESEFVYGDYKVYDVRKMIAKS
EQEIGKATAKYFFYSNIMNFFKTEITLANGEIRKRPLIETNGETGEIVWDKGRDFATVRKVLSM
PQVNIVKKTEVQTGGFSKESILPKRNSDKLIARKKDWDPKKYGGFDSPTVAYSVLVVAKVEKGK
SKKLKSVKELLGITIMERSSFEKNPIDFLEAKGYKEVKKDLIIKLPKYSLFELENGRKRMLASA
GELQKGNELALPSKYVNFLYLASHYEKLKGSPEDNEQKQLFVEQHKHYLDEIIEQISEFSKRVI
LADANLDKVLSAYNKHRDKPIREQAENIIHLFTLTNLGAPAAFKYFDTTIDRKRYTSTKEVLDA
TLIHQSITGLYETRIDLSQLGGDSGGSSGGSSGSETPGTSESATPESSGSGGSSGGSSGSETPG
TSESATPESSGGSSGGSSILKILNEIASIGSTKQKQAILEKNKDNELLKRVYRLTYSRGLQYYI
KKWPKPGIATQSFGMLTLTDMLDFIEFTLATRKLTGNAAIEELTGYITDGKKDDVEVLRRVMMR
DLECGASVSIANKVWPGLIPEQPQMLASSYDEKGINKNIKFPAFAQLKADGARCFAEVRGDELD
DVRLLSRAGNEYLGLDLLKEELIKMTAEARQIHPEGVLIDGELVYHEQVKKEPEGLDFLFDAYP
ENSKAKEFAEVAESRTASNGIANKSLKGTISEKEAQCMKFQVWDYVPLVEIYSLPAFRLKYDVR
FSKLEQMTSGYDKVILIENQVVNNLDEAKVIYKKYIDQGLEGIILKNIDGLWENARSKNLYKFK
EVIDVDLKIVGIYPHRKDRTDYVKLFLPIAIRLREDKTKANTFEDVFGDFHEVTGLPKKKRKV
Amino acid sequence of Cas9-MCP2 MMLV RT
(SEQ ID NO: 24)
PKKKRKVDKKYSIGLDIGTNSVGWAVITDEYKVPSKKFKVLGNTDRHSIKKNLIGALLFDSGET
AEATRLKRTARRRYTRRKNRICYLQEIFSNEMAKVDDSFFHRLEESFLVEEDKKHERHPIFGNI
VDEVAYHEKYPTIYHLRKKLVDSTDKADLRLIYLALAHMIKFRGHFLIEGDLNPDNSDVDKLFI
QLVQTYNQLFEENPINASGVDAKAILSARLSKSRRLENLIAQLPGEKKNGLFGNLIALSLGLTP
NFKSNFDLAEDAKLQLSKDTYDDDLDNLLAQIGDQYADLFLAAKNLSDAILLSDILRVNTEITK
APLSASMTEELLVKLNREDLLRKQRTFDNGSIPHQIHLGELHAILRRQEDFYPFLKDNREKIEK
ILTFRIPYYVGPLARGNSRFAWMTRKSEETITPWNFEEVVDKGASAQSFIERMTNFDKNLPNEK
VLPKHSLLYEYFTVYNELTKVKYVTEGMRKPAFLSGEQKKAIVDLLFKTNRKVTVKQLKEDYFK
KIECFDSVEISGVEDRFNASLGTYHDLLKIIKDKDFLDNEENEDILEDIVLTLTLFEDREMIEE
RLKTYAHLFDDKVMKOLKRRRYTGWGRLSRKLINGIRDKQSGKTILDFLKSDGFANRNFMQLIH
DDSLTFKEDIQKAQVSGQGDSLHEHIANLAGSPAIKKGILQTVKVVDELVKVMGRHKPENIVIE
MARENQTSDYDVDHIVPQSFLKDDSIDNKVLTRSDKNRGKSDNVPSEEVVKKMKNYWRQLLNAK
LITQRKFDNLTKAERGGLSELDKAGFIKRQLVETRQITKHVAQILDSRMNTKYDENDKLIREVK
VITLKSKLVSDFRKDFQFYKVREINNYHHAHDAYLNAVVGTALIKKYPKLESEFVYGDYKVYDV
RKMIAKSEQEIGKATAKYFFYSNIMNFFKTEITLANGEIRKRPLIETNGETGEIVWDKGRDFAT
VRKVLSMPQVNIVKKTEVQTGGFSKESILPKRNSDKLIARKKDWDPKKYGGFDSPTVAYSVLVV
AKVEKGKSKKLKSVKELLGITIMERSSFEKNPIDFLEAKGYKEVKKDLIIKLPKYSLFELENGR
KRMLASAGELQKGNELALPSKYVNFLYLASHYEKLKGSPEDNEQKQLFVEQHKHYLDEIIEQIS
EFSKRVILADANLDKVLSAYNKHRDKPIREQAENIIHLFTLTNLGAPAAFKYFDTTIDRKRYTS
TKEVLDATLIHQSITGLYETRIDLSQLGGDSGGSSGGSSGSETPGTSESATPESSGGSSGGSSA
SNFTQFVLVDNGGTGDVTVAPSNFANGVAEWISSNSRSQAYKVTCSVRQSSAQKRKYTIKVEVP
KVATQTVGGVELPVAAWRSYLNMELTIPIFATNSDCELIVKAMQGLLKDGNPIPSAIAANSGIY
SGGSSGGSSGSETPGTSESATPESSGGSSGGSSTLNIEDEYRLHETSKEPDVSLGSTWLSDFPQ
AWAETGGMGLAVRQAPLIIPLKATSTPVSIKQYPMSQEARLGIKPHIQRLLDQGILVPCQSPWN
TPLLPVKKPGTNDYRPVQDLREVNKRVEDIHPTVPNPYNLLSGLPPSHQWYTVLDLKDAFFCLR
LHPTSQPLFAFEWRDPEMGISGQLTWTRLPQGFKNSPTLFNEALHRDLADFRIQHPDLILLQYV
DDLLLAATSELDCQQGTRALLQTLGNLGYRASAKKAQICQKQVKYLGYLLKEGQRWLTEARKET
VMGQPTPKTPRQLREFLGKAGFCRLFIPGFAEMAAPLYPLTKPGTLFNWGPDQQKAYQEIKQAL
LTAPALGLPDLTKPFELFVDEKQGYAKGVLTQKLGPWRRPVAYLSKKLDPVAAGWPPCLRMVAA
IAVLTKDAGKLTMGQPLVILAPHAVEALVKQPPDRWLSNARMTHYQALLLDTDRVQFGPVVALN
PATLLPLPEEGLQHNCLDILAEAHGTRPDLTDQPLPDADHTWYTDGSSLLQEGORKAGAAVTTE
TEVIWAKALPAGTSAQRAELIALTQALKMAEGKKLNVYTDSRYAFATAHIHGEIYRRRGWLTSE
GKEIKNKDEILALLKALFLPKRLSIIHCPGHQKGHSAEARGNRMADQAARKAAITETPDTSTLL
IENSSPSGGSKRTADGSEFEPKKKRKV
Amino acid sequence of Cas9-T4 DNA Pol
(SEQ ID NO: 25)
PKKKRKVDKKYSIGLDIGTNSVGWAVITDEYKVPSKKFKVLGNTDRHSIKKNLIGALLFDSGET
AEATRLKRTARRRYTRRKNRICYLQEIFSNEMAKVDDSFFHRLEESFLVEEDKKHERHPIFGNI
VDEVAYHEKYPTIYHLRKKLVDSTDKADLRLIYLALAHMIKFRGHFLIEGDLNPDNSDVDKLFI
QLVQTYNQLFEENPINASGVDAKAILSARLSKSRRLENLIAQLPGEKKNGLFGNLIALSLGLTP
NFKSNFDLAEDAKLQLSKDTYDDDLDNLLAQIGDQYADLFLAAKNLSDAILLSDILRVNTEITK
APLSASMTEELLVKLNREDLLRKQRTFDNGSIPHQIHLGELHAILRRQEDFYPFLKDNREKIEK
ILTFRIPYYVGPLARGNSRFAWMTRKSEETITPWNFEEVVDKGASAQSFIERMTNFDKNLPNEK
VLPKHSLLYEYFTVYNELTKVKYVTEGMRKPAFLSGEQKKAIVDLLFKTNRKVTVKQLKEDYFK
KIECFDSVEISGVEDRFNASLGTYHDLLKIIKDKDFLDNEENEDILEDIVLTLTLFEDREMIEE
RLKTYAHLFDDKVMKQLKRRRYTGWGRLSRKLINGIRDKQSGKTILDFLKSDGFANRNFMQLIH
DDSLTFKEDIQKAQVSGQGDSLHEHIANLAGSPAIKKGILQTVKVVDELVKVMGRHKPENIVIE
MARENQTSDYDVDHIVPQSFLKDDSIDNKVLTRSDKNRGKSDNVPSEEVVKKMKNYWRQLLNAK
LITQRKFDNLTKAERGGLSELDKAGFIKRQLVETRQITKHVAQILDSRMNTKYDENDKLIREVK
VITLKSKLVSDFRKDFQFYKVREINNYHHAHDAYLNAVVGTALIKKYPKLESEFVYGDYKVYDV
RKMIAKSEQEIGKATAKYFFYSNIMNFFKTEITLANGEIRKRPLIETNGETGEIVWDKGRDFAT
VRKVLSMPQVNIVKKTEVQTGGFSKESILPKRNSDKLIARKKDWDPKKYGGFDSPTVAYSVLVV
AKVEKGKSKKLKSVKELLGITIMERSSFEKNPIDFLEAKGYKEVKKDLIIKLPKYSLFELENGR
KRMLASAGELQKGNELALPSKYVNFLYLASHYEKLKGSPEDNEQKQLFVEQHKHYLDEIIEQIS
EFSKRVILADANLDKVLSAYNKHRDKPIREQAENIIHLFTLTNLGAPAAFKYFDTTIDRKRYTS
TKEVLDATLIHQSITGLYETRIDLSQLGGDSGGSSGGSSGSETPGTSESATPESSGGSSGGSSP
SMKDARDWMKRMEDIGLEALGMNDFKLAYISDTYGSEIVYDRKFVRVANCDIEVTGDKFPDPMK
AEYEIDAITHYDSIDDRFYVFDLLNSMYGSVSKWDAKLAAKLDCEGGDEVPQEILDRVIYMPFD
NERDMLMEYINLWEQKRPAIFTGWNIEGFDVPYIMNRVKMILGERSMKRFSPIGRVKSKLIQNM
YGSKEIYSIDGVSILDYLDLYKKFAFTNLPSFSLESVAQHETKKGKLPYDGPINKLRETNHQRY
ISYNIIDVESVQAIDKIRGFIDLVLSMSYYAKMPFSGVMSPIKTWDAIIFNSLKGEHKVIPQQG
SHVKQSFPGAFVFEPKPIARRYIMSFDLTSLYPSIIRQVNISPETIRGQFKVHPIHEYIAGTAP
KPSDEYSCSPNGWMYDKHQEGIIPKEIAKVFFQRKDWKKKMFAEEMNAEAIKKIIMKGAGSCST
KPEVERYVKFSDDFLNELSNYTESVLNSLIEECEKAATLANTNOLNRKILINSLYGALGNIHFR
YYDLRNATAITIFGQVGIQWIARKINEYLNKVCGTNDEDFIAAGDTDSVYVCVDKVIEKVGLDR
FKEQNDLVEFMNQFGKKKMEPMIDVAYRELCDYMNNREHLMHMDREAISCPPLGSKGVGGFWKA
KKRYALNVYDMEDKRFAEPHLKIMGMETQQSSTPKAVQEALEESIRRILQEGEESVQEYYKNFE
KEYROLDYKVIAEVKTANDIAKYDDKGWPGFKCPFHIRGVLTYRRAVSGLGVAPILDGNKVMVL
PLREGNPFGDKCIAWPSGTELPKEIRSDVLSWIDHSTLFQKSFVKPLAGMCESAGMDYEEKASL
DFLFGPKKKRKV
Amino acid sequence of T4gp32-FUBP
(SEQ ID NO: 26)
PKKKRKVMFKRKSTAELAAQMAKLNGNKGFSSEDKGEWKLKLDNAGNGQAVIRFLPSKNDEQAP
FAILVNHGFKKNGKWYIETCSSTHGDYDSCPVCQYISKNDLYNTDNKEYSLVKRKTSYWANILV
VKDPAAPENEGKVFKYRFGKKIWDKINAMIAVDVEMGETPVDVTCPWEGANFVLKVKQVSGFSN
YDESKFLNQSAIPNIDDESFQKELFEQMVDLSEMTSKDKFKSFEELNTKFGQVMGTAVMGGAAA
TAAKKADKVADDLDAFNVDDFNTKTEDDFMSSSSGSSSSADDTDLDDLLNDLMADYSTVPPPSS
GSAGGGGSFGTQLPPMHQQQSRSVMTEEYKVPDGMVGFIIGRGGEQISRIQQESGCKIQIAPDS
GGLPERSCMLTGTPESVQSAKRLLDQIVEKGRPAPGFHHGDGPGNAVQEIMIPASKAGLVIGKG
GETIKQLQERAGVKMVMIQDGPQNTGADKPLRITGDPYKVQQAKEMVLELIRDOGGFREVRNEY
GSRIGGNEGIDVPIPRFAVGIVIGRNGEMIKKIQNDAGVRIQFKPDDGTTPERIAQITGPPDRC
QHAAEIITDLLRSVQAGNPGGPGPGGRGRGRGQGNWNMGPPGGLQEFNFIVPTGKTGLIIGKGG
ETIKSISQQSGARIELQRNPPPNADPNMKLFTIRGTPQQIDYARQLIEEKIGGPVNPLGPPVPH
GPHGVPGPHGPPGPPGPGTPMGPYNPAPYNPGPPGPAPHGPPAPYAPQGWGNAYPHWQQQAPPD
PAKAGTDPNSAAWAAYYAHYYQQQAQPPPAAPAGAPTTTQTNGQGDQQNPAPAGQVDYTKAWEE
YYKKMGQAVPAPTGAPPGGQPDYSAAWAEYYRQQAAYYAQTSPQGMPQHPPAPQGQ
Polynucleotide sequence of AAVS 123 AAGATG gRNA
(SEQ ID NO: 27)
AGAGGGCCTATTTCCCATGATTCCTTCATATTTGCATATACGATACAAGGCTGTTAGAGAGATA
ATTAGAATTAATTTGACTGTAAACACAAAGATATTAGTACAAAATACGTGACGTAGAAAGTAAT
AATTTCTTGGGTAGTTTGCAGTTTTAAAATTATGTTTTAAAATGGACTATCATATGCTTACCGT
AACTTGAAAGTATTTCGATTTCTTGGCTTTATATATCTTGTGGAAAGGACGAAACACCGTGGCC
CCACTGTGGGGTGGGTTTTAGAGCTAGAAATAGCAAGTTAAAATAAGGCTAGTCCGTTATCAAC
TTGAAAAAGTGGGACCGAGTCGGTCCAAGATGCCCCACAGTTTTTTTT
Polynucleotide sequence of AAVS 123 AAGATG 20
extension gRNA
(SEQ ID NO: 28)
AGAGGGCCTATTTCCCATGATTCCTTCATATTTGCATATACGATACAAGGCTGTTAGAGAGATA
ATTAGAATTAATTTGACTGTAAACACAAAGATATTAGTACAAAATACGTGACGTAGAAAGTAAT
AATTTCTTGGGTAGTTTGCAGTTTTAAAATTATGTTTTAAAATGGACTATCATATGCTTACCGT
AACTTGAAAGTATTTCGATTTCTTGGCTTTATATATCTTGTGGAAAGGACGAAACACCGTGGCC
CCACTGTGGGGTGGGTTTTAGAGCTAGAAATAGCAAGTTAAAATAAGGCTAGTCCGTTATCAAC
TTGAAAAAGTGGGACCGAGTCGGTCCAAGATGCCCCACAGTGGGGCCACTAGTTTTTTT

What is claimed is:

1. A fusion protein comprising: (i) a Cas nuclease and (ii) a reverse transcriptase, a DNA polymerase, a DNA ligase, or a combination thereof, wherein the Cas nuclease is capable of generating a double-stranded polynucleotide cleavage.

2. The fusion protein of claim 1, wherein the Cas nuclease is Cas9, Cas12, or Cas14.

3. The fusion protein of claim 2, wherein the Cas nuclease comprises a polypeptide sequence having at least 90% identity to any one of SEQ ID NOS: 1, 29, or 30.

4. The fusion protein of claim 2, wherein the Cas9 is a Type IIB Cas9.

5. The fusion protein of claim 1, wherein the fusion protein comprises a Cas nuclease and a reverse transcriptase.

6. The fusion protein of claim 5, wherein the reverse transcriptase is MMLV reverse transcriptase or R2 reverse transcriptase.

7. The fusion protein of claim 5 or 6, wherein the reverse transcriptase comprises a polypeptide sequence having at least 90% identity to any one of SEQ ID NOS: 2-3.

8. The fusion protein of claim 1, wherein the fusion protein comprises a Cas nuclease and a DNA polymerase.

9. The fusion protein of claim 7, wherein the DNA polymerase is phi29 DNA polymerase, T4 DNA polymerase, DNA polymerase mu, DNA polymerase delta, or DNA polymerase epsilon.

10. The fusion protein of claim 7 or 8, wherein the DNA polymerase comprises a polypeptide sequence having at least 90% identity to any one of SEQ ID NOS: 4-6.

11. The fusion protein of claim 1, wherein the fusion protein comprises a Cas nuclease and a DNA ligase.

12. The fusion protein of claim 11, wherein the DNA ligase is T4 DNA ligase.

13. The fusion protein of claim 11 or 12, wherein the DNA ligase comprises a polypeptide sequence having at least 90% identity to SEQ ID NO: 7.

14. The fusion protein of any one of claims 1 to 13, further comprising a DNA-binding or an RNA-binding domain.

15. The fusion protein of claim 14, wherein the DNA-binding domain is a zinc finger DNA-binding domain, a transcription factor, or an adeno-associated virus Rep protein.

16. The fusion protein of claim 14, wherein the RNA-binding domain is MS2 coat protein (MCP2).

17. The fusion protein of claim 14, wherein the RNA-binding domain comprises a KH domain.

18. The fusion protein of claim 17, wherein the RNA-binding domain is heterogeneous nuclear ribonucleoprotein K (hnRNPK).

19. The fusion protein of claim 14, wherein the DNA-binding domain is capable of binding single-stranded DNA (ssDNA).

20. The fusion protein of claim 19, wherein DNA-binding domain is Far upstream element-binding protein (FUBP).

21. The fusion protein of any one of claims 14 to 20, wherein the DNA-binding or the RNA-binding domain comprises a polypeptide sequence having at least 90% identity to any one of SEQ ID NOS: 8-11.

22. The fusion protein of any one of claims 1 to 21, further comprising a polypeptide linker between (i) and (ii).

23. The fusion protein of claim 1, comprising a polypeptide sequence having at least 90% identity to any one of SEQ ID NOS: 18-26.

24. A composition comprising:
a) the fusion protein of any one of claims 1 to 23; and
b) a polynucleotide that forms a complex with the fusion protein and comprises (i) a guide sequence; and (ii) a template sequence for the reverse transcriptase, the DNA polymerase, or the DNA ligase.

25. The composition of claim 24, wherein the polynucleotide comprises RNA.

26. The composition of claim 24, wherein the guide sequence comprises RNA and the template sequence comprises DNA.

27. The composition of claim 24, wherein the template sequence comprises an abasic site, a triethylene glycol (TEG) linker, or both.

28. The composition of any one of claims 24 to 27, wherein the guide sequence is about 15 to about 20 nucleotides in length.

29. The composition of any one of claims 24 to 28, wherein the polynucleotide further comprises a tracrRNA.

30. The composition of any one of claims 24 to 28, wherein the composition comprises a second polynucleotide comprising a tracrRNA.

31. The composition of any one of claims 24 to 30, wherein the template sequence comprises a primer-binding sequence and a sequence of interest.

32. The composition of claim 31, wherein the primer-binding sequence and the sequence of interest comprise DNA.

33. The composition of claim 31, wherein the sequence of interest comprises DNA.

34. The composition of any one of claims 24 to 33, wherein the template sequence is about 25 to about 10000 nucleotides in length.

35. The composition of any one of claims 24 to 34, wherein the primer-binding sequence is about 4 to about 30 nucleotides in length.

36. The composition of any one of claims 24 to 35, wherein the sequence of interest is about 5 nucleotides to about 9000 nucleotides in length.

37. The composition of any one of claims 24 to 36, wherein the polynucleotide comprises a spacer between the guide sequence and the template sequence.

38. The composition of claim 37, wherein the spacer is about 10 to about 200 nucleotides in length.

39. The composition of claim 37 or 38, wherein the spacer comprises a stop sequence for the reverse transcriptase or DNA polymerase.

40. The composition of claim 39, wherein the spacer comprises more than one stop sequence.

41. The composition of claim 39 or 40, wherein the stop sequence comprises a secondary structure.

42. The composition of claim 41, wherein the secondary structure is a hairpin loop.

43. A composition comprising:
a) the fusion protein of any one of claims 1 to 23;
b) a guide polynucleotide that forms a complex with the fusion protein and comprises a guide sequence; and
c) a template polynucleotide comprising a template sequence for the reverse transcriptase, the DNA polymerase, or the DNA ligase.

44. The composition of claim 43, wherein the guide polynucleotide is RNA.

45. The composition of claim 43, wherein the template polynucleotide comprises RNA.

46. The composition of claim 43, wherein the template sequence comprises DNA.

47. The composition of claim 43, wherein the template sequence comprises an abasic site, a triethylene glycol (TEG) linker, or both.

48. The composition of any one of claims 43 to 47, wherein the guide sequence is about 15 to about 20 nucleotides in length.

49. The composition of any one of claims 43 to 48, wherein the guide polynucleotide further comprises a tracrRNA.

50. The composition of any one of claims 43 to 48, wherein the composition further comprises a third polynucleotide comprising a tracrRNA.

51. The composition of any one of claims 43 to 50, wherein the template sequence is about 25 to about 10000 nucleotides in length.

52. The composition of any one of claims 43 to 51, wherein the template sequence comprises a sequence of interest.

53. The composition of claim 52, wherein the sequence of interest is about 5 nucleotides to about 9800 nucleotides in length.

54. The composition of claim 52 or 53, wherein the sequence of interest comprises DNA.

55. The composition of any one of claims 43 to 54, wherein the template polynucleotide further comprises a primer-binding sequence.

56. The composition of claim 55, wherein the primer-binding sequence is about 4 to about 30 nucleotides in length.

57. The composition of claim 55 or 56, wherein the primer-binding sequence and the sequence of interest comprise DNA.

58. The composition of any one of claims 43 to 57, wherein the template polynucleotide further comprises a stop sequence for the reverse transcriptase or DNA polymerase.

59. The composition of claim 58, wherein the template polynucleotide comprises more than one stop sequence.

60. The composition of claim 58 or 59, wherein the stop sequence comprises a secondary structure.

61. The composition of claim 60, wherein the secondary structure is a hairpin loop.

62. The composition of any one of claims 43 to 61, where the template polynucleotide comprises an adeno-associated virus (AAV) vector comprising a sequence of interest.

63. A polynucleotide encoding the fusion protein of any one of claims 1 to 23.

64. A vector comprising the polynucleotide encoding the fusion protein of claims 1 to 23.

65. A cell comprising the fusion protein of any one of claims 1 to 23.

66. A cell comprising the polynucleotide encoding the fusion protein of claims 1 to 23, or the vector of claim 64.

67. A cell comprising the composition of any one of claims 24 to 62.

68. A method of providing a site-specific modification at a target sequence in a target polynucleotide, the method comprising contacting the target polynucleotide with the composition of any one of claims 24 to 62.

69. The method of claim 68, wherein the target polynucleotide is DNA.

70. The method of claim 68 or 69, wherein the guide sequence is capable of hybridizing to the target sequence.

71. The method of any one of claims 68 to 70, wherein the contacting is performed under conditions sufficient for the Cas nuclease to generate a double-stranded polynucleotide cleavage at the target sequence.

72. The method of any one of claims 68 to 71, wherein the template sequence comprises a sequence of interest.

73. The method of any one of claims 68 to 72, wherein the template sequence comprises a primer-binding sequence capable of hybridizing to the target sequence.

74. The method of any one of claims 68 to 73, wherein the contacting is performed under conditions sufficient for the reverse transcriptase to transcribe a complementary strand of the sequence of interest.

75. The method of claim 74, further comprising cleaving the template sequence to generate a double-stranded sequence comprising the sequence of interest.

76. The method of claim 75, wherein the cleaving is performed by RNase H.

77. The method of any one of claims 68 to 72, wherein the contacting is performed under conditions sufficient for the DNA polymerase to generate a double-stranded sequence comprising the sequence of interest.

78. The method of any one of claims 68 to 72, wherein the contacting is performed under conditions sufficient for the DNA ligase to ligate the sequence of interest to the cleaved target sequence.

79. The method of any one of claims 71 to 78, wherein the double-stranded sequence comprising the sequence of interest is inserted into the cleaved target sequence by non-homologous end joining (NHEJ).

80. The method of any one of claims 71 to 78, wherein the double-stranded sequence comprising the sequence of interest is inserted into the cleaved target sequence by a DNA ligase.

81. The method of any one of claims 68 to 77, further comprising generating a second double-stranded polynucleotide cleavage at a second target sequence in the target polynucleotide.

82. The method of claim 81, wherein the sequence of interest replaces a sequence of the target polynucleotide between the target sequence and the second target sequence.

83. A kit comprising the fusion protein of any one of claims 1 to 23.

84. The kit of claim 83, further comprising a polynucleotide that forms a complex with the fusion protein and/or a vector for expressing the polynucleotide.

85. The kit of claim 83, further comprising a template polynucleotide comprising a template sequence for the reverse transcriptase, the DNA polymerase, or the DNA ligase and/or a vector for expressing the template polynucleotide.

86. The kit of claim 83 or 84, further comprising a polynucleotide comprising a tracrRNA.

87. The kit of any one of claims 83 to 86, further comprising RNase H.


SEQUENCE LISTING (48 sequences)

SEQ ID NO: 1 - protein, 1367 aa, Artificial Sequence
  source: /note=“Description of Artificial Sequence Synthetic polypeptide”
DKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLNIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 2 - protein, 691 aa, Artificial Sequence
  source: /note=“Description of Artificial Sequence Synthetic polypeptide”
TLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSPSGGSKRTADGSEFE

SEQ ID NO: 3 - protein, 1275 aa, Artificial Sequence
  source: /note=“Description of Artificial Sequence Synthetic polypeptide”
GTDTVYVGQDYPSGLSKRVPARLVAGPMLRERSCHAHVFRAGHMWNWRTSLPSGRWDQPALEKSRVLTRSVATATDPEITSYPGKSVSTSTQVQEEDWCSRESGWISPGLAPEEPSVVSEITASMVATMRVATEEVVLEPQPEQVVTILPEHGRNVPPGLAEQDTASPIEVSVLLPDLAENCPLCGVPSGGLRLLGKHFAVRHAGVPVTYECRKCAWRSPNSHSISCHVPKCRGRARMPSGDPGIACDLCEARFATEVGVAQHKRHVHPVEWNKVRLERRGARGGGIKATKLWSVAEVETLIRLIREHGDSGATYQLIADELGRGKTAEQVRSKKRLLRIDTASNSPDDAEVEEERLESLAVRSSSRSPPSLVATRVREAVARGESEGGEEIRAIAALIRDVDQNPCLIETSASDIISKLGRRVDGPKRPRPVVREQTQEKGWVRRLARRKREYREAQYLYSRDQARLAAQILDGAASQECALPVDQVYGAFREKWETVGQFHGLGEFRTGARADNWEFYSPILAAEVKENLMRMANGTAPGPDRISKKALLDWDPRGEQLARLYTTWLIGGVIPRVFKECRTKLLPKSSDPVELQDIGGWRPVTIGSMVTRLFSRILTMRLTRACPINPRQRGFLASSSGCAENLLIFDEIVRRSRRDGGPLAVVFVDFARAFDSISHEHILCVLEEGGLDRHVIGLIRNSYVDCVTRVGCVEGMTPPIQMKVGVKQGDPMSPLLFNLAMDPLIHKLETAGTGLKWGDLSIATLAFADDLVLVSDSEEGMGRSLGILEKFCQLTGLRVQPRKCHGFFMDKGVVNGCGTWEICGSPIHMIPPGESVRYLGVQVGPGRGVMEPDLIPTVHTWIERISEAPLKPSQRMRVLNSFALPRIIYQADLGKVTVTKLAQIDGIVRKAVKKWLHLSPSTCNGLLYSRNRDGGLGLLKLERLIPSVRTKRIYRMSRSPDIWTRRMTSHSVSKSDWEMLWVQAGGERGSAPVMGAVEAAPTDVERSPDYPDWRREENLAWSALRVQGVGADQFRGDRTSSSWIAEPASVGFAQRHWLAALALRAGVYPTREFLARGKEKSGAACRRCPARLESCSHILGQCPFVQANRIARHNKVCVLLATEAERFGWTVIREFRLEDAAGGLKIPDLVCKKADTVLIVDVTVRYEMDGETLKRAASEKVKHYLPVGQQITDKVGGRCFKVMGFPVGARGKWPASNNTVLAELGVPAGRMRTFARLVSRRTLLYSLDILRDFMREPAGRGTRVALIPAATGAAN

SEQ ID NO: 4 - protein, 571 aa, Artificial Sequence
  source: /note=“Description of Artificial Sequence Synthetic polypeptide”
PRKMYSCDFETTTKVEDCRVWAYGYMNIEDHSEYKIGNSLDEFMAWVLKVQADLYFHNLKFDGAFIINWLERNGFKWSADGLPNTYNTIISRMGQWYMIDICLGYKGKRKIHTVIYDSLKKLPFPVKKIAKDFKLTVLKGDIDYHKERPVGYKITPEEYAYIKNDIQIIAEALLIQFKQGLDRMTAGSDSLKGFKDIITTKKFKKVFPTLSLGLDKEVRYAYRGGFTWLNDRFKEKEIGEGMVFDVNSLYPAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTGLFKDFIDKWTYIKTTSEGAIKQLAKLMLNSLYGKFASNPDVTGKVPYLKENGALGFRLGEEETKDPVYTPMGVFITAWARYTTITAAQACYDRIIYCDTDSIHLTGTEIPDVIKDIVDPKKLGYWAHESTFKRAKYLRQKTYIQDIYMKEVDGKLVEGSPDDYTDIKFSVKCAGMTDKIKKEVTFENFKVGFSRKMKPKPVQVPGGVVLVDDTFTIK

SEQ ID NO: 5 - protein, 1106 aa, Artificial Sequence
  source: /note=“Description of Artificial Sequence Synthetic polypeptide”
DGKRRPGPGPGVPPKRARGGLWDDDDAPRPSQFEEDLALMEEMEAEHRLQEQEEEELQSVLEGVADGQVPPSAIDPRWLRPTPPALDPQTEPLIFQQLEIDHYVGPAQPVPGGPPPSHGSVPVLRAFGVTDEGFSVCCHIHGFAPYFYTPAPPGFGPEHMGDLQRELNLAINRDSRGGRELTGPAVLAVELCSRESMFGYHGHGPSPFLRITVALPRLVAPARRLLEQGIRVAGLGTPSFAPYEANVDFEIRFMVDTDIVGCNWLELPAGKYALRLKEKATQCQLEADVLWSDVVSHPPEGPWQRIAPLRVLSFDIECAGRKGIFPEPERDPVIQICSLGLRWGEPEPFLRLALTLRPCAPILGAKVQSYEKEEDLLQAWSTFIRIMDPDVITGYNIQNFDLPYLISRAQTLKVQTFPFLGRVAGLCSNIRDSSFQSKQTGRRDTKVVSMVGRVQMDMLQVLLREYKLRSYTLNAVSFHFLGEQKEDVQHSIITDLQNGNDQTRRRLAVYCLKDAYLPLRLLERLMVLVNAVEMARVTGVPLSYLLSRGQQVKVVSQLLRQAMHEGLLMPVVKSEGGEDYTGATVIEPLKGYYDVPIATLDFSSLYPSIMMAHNLCYTTLLRPGTAQKLGLTEDQFIRTPTGDEFVKTSVRKGLLPQILENLLSARKRAKAELAKETDPLRRQVLDGRQLALKVSANSVYGFTGAQVGKLPCLEISQSVTGFGRQMIEKTKQLVESKYTVENGYSTSAKVVYGDTDSVMCRFGVSSVAEAMALGGEAADWVSGHFPSPIRLEFEKVYFPYLLISKKRYAGLLFSSRPDAHDRMDCKGLEAVRRDNCPLVANLVTASLRRLLIDRDPEGAVAHAQDVISDLLCNRIDISQLVITKELTRAASDYAGKQAHVELAERMRKRDPGSAPSLGDRVPYVIISAAKGVAAYMKSEDPLFVLEHSLPIDTQYYLEQQLAKPLLRIFEPILGEGRAEAVLLRGDHTRCKTVLTGKVGGLLAFAKRRNCCIGCRTVLSHQGAVCEFCQPRESELYQKEVSHLNALEERFSRLWTQCQRCQGSLHEDVICTSRDCPIFYMRKKVRKDLEDQEQLLRRFGPPGPEAW

SEQ ID NO: 6 - protein, 838 aa, Artificial Sequence
  source: /note=“Description of Artificial Sequence Synthetic polypeptide”
PSMKDARDWMKRMEDIGLEALGMNDFKLAYISDTYGSEIVYDRKFVRVANCDIEVTGDKFPDPMKAEYEIDAITHYDSIDDRFYVFDLLNSMYGSVSKWDAKLAAKLDCEGGDEVPQEILDRVIYMPFDNERDMLMEYINLWEQKRPAIFTGWNIEGFDVPYIMNRVKMILGERSMKRFSPIGRVKSKLIQNMYGSKEIYSIDGVSILDYLDLYKKFAFTNLPSFSLESVAQHETKKGKLPYDGPINKLRETNHQRYISYNIIDVESVQAIDKIRGFIDLVLSMSYYAKMPFSGVMSPIKTWDAIIFNSLKGEHKVIPQQGSHVKQSFPGAFVFEPKPIARRYIMSFDLTSLYPSIIRQVNISPETIRGQFKVHPIHEYIAGTAPKPSDEYSCSPNGWMYDKHQEGIIPKEIAKVFFQRKDWKKKMFAEEMNAEAIKKIIMKGAGSCSTKPEVERYVKFSDDFLNELSNYTESVLNSLIEECEKAATLANTNQLNRKILINSLYGALGNIHFRYYDLRNATAITIFGQVGIQWIARKINEYLNKVCGTNDEDFIAAGDTDSVYVCVDKVIEKVGLDRFKEQNDLVEFMNQFGKKKMEPMIDVAYRELCDYMNNREHLMHMDREAISCPPLGSKGVGGFWKAKKRYALNVYDMEDKRFAEPHLKIMGMETQQSSTPKAVQEALEESIRRILQEGEESVQEYYKNFEKEYRQLDYKVIAEVKTANDIAKYDDKGWPGFKCPFHIRGVLTYRRAVSGLGVAPILDGNKVMVLPLREGNPFGDKCIAWPSGTELPKEIRSDVLSWIDHSTLFQKSFVKPLAGMCESAGMDYEEKASLDFLFG

SEQ ID NO: 7 - protein, 486 aa, Artificial Sequence
  source: /note=“Description of Artificial Sequence Synthetic polypeptide”
ILKILNEIASIGSTKQKQAILEKNKDNELLKRVYRLTYSRGLQYYIKKWPKPGIATQSFGMLTLTDMLDFIEFTLATRKLTGNAAIEELTGYITDGKKDDVEVLRRVMMRDLECGASVSIANKVWPGLIPEQPQMLASSYDEKGINKNIKFPAFAQLKADGARCFAEVRGDELDDVRLLSRAGNEYLGLDLLKEELIKMTAEARQIHPEGVLIDGELVYHEQVKKEPEGLDFLFDAYPENSKAKEFAEVAESRTASNGIANKSLKGTISEKEAQCMKFQVWDYVPLVEIYSLPAFRLKYDVRFSKLEQMTSGYDKVILIENQVVNNLDEAKVIYKKYIDQGLEGIILKNIDGLWENARSKNLYKFKEVIDVDLKIVGIYPHRKDPTKAGGFILESECGKIKVNAGSGLKDKAGVKSHELDRTRIMENQNYYIGKILECECNGWLKSDGRTDYVKLFLPIAIRLREDKTKANTFEDVFGDFHEVTGL

SEQ ID NO: 8 - protein, 129 aa, Artificial Sequence
  source: /note=“Description of Artificial Sequence Synthetic polypeptide”
ASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIY

SEQ ID NO: 9 - protein, 196 aa, Artificial Sequence
  source: /note=“Description of Artificial Sequence Synthetic polypeptide”
PGFYEIVIKVPSDLDGHLPGISDSFVNWVAEKEWELPPDSDMDLNLIEQAPLTVAEKLQRDFLTEWRRVSKAPEALFFVQFEKGESYFHMHVLVETTGVKSMVLGRFLSQIREKLIQRIYRGIEPTLPNWFAVTKTRNGAGGGNKVVDECYIPNFLLPKTQPELQWAWTNMEQYLSACLNLTERKRLVAQHLTHVS

SEQ ID NO: 10 - protein, 301 aa, Artificial Sequence
  source: /note=“Description of Artificial Sequence Synthetic polypeptide”
MFKRKSTAELAAQMAKLNGNKGFSSEDKGEWKLKLDNAGNGQAVIRFLPSKNDEQAPFAILVNHGFKKNGKWYIETCSSTHGDYDSCPVCQYISKNDLYNTDNKEYSLVKRKTSYWANILVVKDPAAPENEGKVFKYRFGKKIWDKINAMIAVDVEMGETPVDVTCPWEGANFVLKVKQVSGFSNYDESKFLNQSAIPNIDDESFQKELFEQMVDLSEMTSKDKFKSFEELNTKFGQVMGTAVMGGAAATAAKKADKVADDLDAFNVDDFNTKTEDDFMSSSSGSSSSADDTDLDDLLNDL

SEQ ID NO: 11 - protein, 644 aa, Artificial Sequence
  source: /note=“Description of Artificial Sequence Synthetic polypeptide”
MADYSTVPPPSSGSAGGGGGGGGGGGVNDAFKDALQRARQIAAKIGGDAGTSLNSNDYGYGGQKRPLEDGDQPDAKKVAPQNDSFGTQLPPMHQQQSRSVMTEEYKVPDGMVGFIIGRGGEQISRIQQESGCKIQIAPDSGGLPERSCMLTGTPESVQSAKRLLDQIVEKGRPAPGFHHGDGPGNAVQEIMIPASKAGLVIGKGGETIKQLQERAGVKMVMIQDGPQNTGADKPLRITGDPYKVQQAKEMVLELIRDQGGFREVRNEYGSRIGGNEGIDVPIPRFAVGIVIGRNGEMIKKIQNDAGVRIQFKPDDGTTPERIAQITGPPDRCQHAAEIITDLLRSVQAGNPGGPGPGGRGRGRGQGNWNMGPPGGLQEFNFIVPTGKTGLIIGKGGETIKSISQQSGARIELQRNPPPNADPNMKLFTIRGTPQQIDYARQLIEEKIGGPVNPLGPPVPHGPHGVPGPHGPPGPPGPGTPMGPYNPAPYNPGPPGPAPHGPPAPYAPQGWGNAYPHWQQQAPPDPAKAGTDPNSAAWAAYYAHYYQQQAQPPPAAPAGAPTTTQTNGQGDQQNPAPAGQVDYTKAWEEYYKKMGQAVPAPTGAPPGGQPDYSAAWAEYYRQQAAYYAQTSPQGMPQHPPAPQGQ

SEQ ID NO: 12 - protein, 19 aa, Artificial Sequence
  source: /note=“Description of Artificial Sequence Synthetic peptide”
MKRTADGSEFESPKKKRKV

SEQ ID NO: 13 - protein, 21 aa, Artificial Sequence
  source: /note=“Description of Artificial Sequence Synthetic peptide”
SGGSKRTADGSEFEPKKKRKV

SEQ ID NO: 14 - protein, 7 aa, Simian virus 40
PKKKRKV

SEQ ID NO: 15 - protein, 32 aa, Artificial Sequence
  source: /note=“Description of Artificial Sequence Synthetic polypeptide”
SGGSSGGSSGSETPGTSESATPESSGGSSGGS

SEQ ID NO: 16 - protein, 26 aa, Artificial Sequence
  source: /note=“Description of Artificial Sequence Synthetic peptide”
SGGSSGGSSGSETPGTSESATPESSG

SEQ ID NO: 17 - protein, 1907 aa, Artificial Sequence
  source: /note=“Description of Artificial Sequence Synthetic polypeptide”
MKRTADGSEFESPKKKRKVPGFYEIVIKVPSDLDGHLPGISDSFVNWVAEKEWELPPDSDMDLNLIEQAPLTVAEKLQRDFLTEWRRVSKAPEALFFVQFEKGESYFHMHVLVETTGVKSMVLGRFLSQIREKLIQRIYRGIEPTLPNWFAVTKTRNGAGGGNKVVDECYIPNFLLPKTQPELQWAWTNMEQYLSACLNLTERKRLVAQHLTHVSSGGSSGGSSGSETPGTSESATPESSGGSSGGSDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLNIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSKRTADGSEFEPKKKRKVGSGATNFSLLKQAGDVEENPGPMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKSGGSPKKKRKV

SEQ ID NO: 18 - protein, 2105 aa, Artificial Sequence
  source: /note=“Description of Artificial Sequence Synthetic polypeptide”
PKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSSGSETPGTSESATPESSGGSSGGSSTLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSPSGGSKRTADGSEFEPKKKRKV

SEQ ID NO: 19 - protein, 834 aa, Artificial Sequence
  source: /note=“Description of Artificial Sequence Synthetic polypeptide”
ASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIYPKKKRKVTLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSPSGGSKRTADGSEFEPKKKRKV

SEQ ID NO: 20 - protein, 1985 aa, Artificial Sequence
  source: /note=“Description of Artificial Sequence Synthetic polypeptide”
PKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSSGSETPGTSESATPESSGGSSGGSSPRKMYSCDFETTTKVEDCRVWAYGYMNIEDHSEYKIGNSLDEFMAWVLKVQADLYFHNLKFDGAFIINWLERNGFKWSADGLPNTYNTIISRMGQWYMIDICLGYKGKRKIHTVIYDSLKKLPFPVKKIAKDFKLTVLKGDIDYHKERPVGYKITPEEYAYIKNDIQIIAEALLIQFKQGLDRMTAGSDSLKGFKDIITTKKFKKVFPTLSLGLDKEVRYAYRGGFTWLNDRFKEKEIGEGMVFDVNSLYPAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTGLFKDFIDKWTYIKTTSEGAIKQLAKLMLNSLYGKFASNPDVTGKVPYLKENGALGFRLGEEETKDPVYTPMGVFITAWARYTTITAAQACYDRIIYCDTDSIHLTGTEIPDVIKDIVDPKKLGYWAHESTFKRAKYLRQKTYIQDIYMKEVDGKLVEGSPDDYTDIKFSVKCAGMTDKIKKEVTFENFKVGFSRKMKPKPVQVPGGVVLVDDTFTIKPKKKRKV

SEQ ID NO: 21 - protein, 2546 aa, Artificial Sequence
  source: /note=“Description of Artificial Sequence Synthetic polypeptide”
PKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSSGSETPGTSESATPESSGDGKRRPGPGPGVPPKRARGGLWDDDDAPRPSQFEEDLALMEEMEAEHRLQEQEEEELQSVLEGVADGQVPPSAIDPRWLRPTPPALDPQTEPLIFQQLEIDHYVGPAQPVPGGPPPSHGSVPVLRAFGVTDEGFSVCCHIHGFAPYFYTPAPPGFGPEHMGDLQRELNLAINRDSRGGRELTGPAVLAVELCSRESMFGYHGHGPSPFLRITVALPRLVAPARRLLEQGIRVAGLGTPSFAPYEANVDFEIRFMVDTDIVGCNWLELPAGKYALRLKEKATQCQLEADVLWSDVVSHPPEGPWQRIAPLRVLSFDIECAGRKGIFPEPERDPVIQICSLGLRWGEPEPFLRLALTLRPCAPILGAKVQSYEKEEDLLQAWSTFIRIMDPDVITGYNIQNFDLPYLISRAQTLKVQTFPFLGRVAGLCSNIRDSSFQSKQTGRRDTKVVSMVGRVQMDMLQVLLREYKLRSYTLNAVSFHFLGEQKEDVQHSIITDLQNGNDQTRRRLAVYCLKDAYLPLRLLERLMVLVNAVEMARVTGVPLSYLLSRGQQVKVVSQLLRQAMHEGLLMPVVKSEGGEDYTGATVIEPLKGYYDVPIATLDFSSLYPSIMMAHNLCYTTLLRPGTAQKLGLTEDQFIRTPTGDEFVKTSVRKGLLPQILENLLSARKRAKAELAKETDPLRRQVLDGRQLALKVSANSVYGFTGAQVGKLPCLEISQSVTGFGRQMIEKTKQLVESKYTVENGYSTSAKVVYGDTDSVMCRFGVSSVAEAMALGGEAADWVSGHFPSPIRLEFEKVYFPYLLISKKRYAGLLFSSRPDAHDRMDCKGLEAVRRDNCPLVANLVTASLRRLLIDRDPEGAVAHAQDVISDLLCNRIDISQLVITKELTRAASDYAGKQAHVELAERMRKRDPGSAPSLGDRVPYVIISAAKGVAAYMKSEDPLFVLEHSLPIDTQYYLEQQLAKPLLRIFEPILGEGRAEAVLLRGDHTRCKTVLTGKVGGLLAFAKRRNCCIGCRTVLSHQGAVCEFCQPRESELYQKEVSHLNALEERFSRLWTQCQRCQGSLHEDVICTSRDCPIFYMRKKVRKDLEDQEQLLRRFGPPGPEAWSGGSSGGSSGSETPGTSESATPESSGGSSGGSSPKKKRKV

SEQ ID NO: 22 - protein, 2689 aa, Artificial Sequence
  source: /note=“Description of Artificial Sequence Synthetic polypeptide”
PKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSSGSETPGTSESATPESSGGSSGGSSGTDTVYVGQDYPSGLSKRVPARLVAGPMLRERSCHAHVFRAGHMWNWRTSLPSGRWDQPALEKSRVLTRSVATATDPEITSYPGKSVSTSTQVQEEDWCSRESGWISPGLAPEEPSVVSEITASMVATMRVATEEVVLEPQPEQVVTILPEHGRNVPPGLAEQDTASPIEVSVLLPDLAENCPLCGVPSGGLRLLGKHFAVRHAGVPVTYECRKCAWRSPNSHSISCHVPKCRGRARMPSGDPGIACDLCEARFATEVGVAQHKRHVHPVEWNKVRLERRGARGGGIKATKLWSVAEVETLIRLIREHGDSGATYQLIADELGRGKTAEQVRSKKRLLRIDTASNSPDDAEVEEERLESLAVRSSSRSPPSLVATRVREAVARGESEGGEEIRAIAALIRDVDQNPCLIETSASDIISKLGRRVDGPKRPRPVVREQTQEKGWVRRLARRKREYREAQYLYSRDQARLAAQILDGAASQECALPVDQVYGAFREKWETVGQFHGLGEFRTGARADNWEFYSPILAAEVKENLMRMANGTAPGPDRISKKALLDWDPRGEQLARLYTTWLIGGVIPRVFKECRTKLLPKSSDPVELQDIGGWRPVTIGSMVTRLFSRILTMRLTRACPINPRQRGFLASSSGCAENLLIFDEIVRRSRRDGGPLAVVFVDFARAFDSISHEHILCVLEEGGLDRHVIGLIRNSYVDCVTRVGCVEGMTPPIQMKVGVKQGDPMSPLLFNLAMDPLIHKLETAGTGLKWGDLSIATLAFADDLVLVSDSEEGMGRSLGILEKFCQLTGLRVQPRKCHGFFMDKGVVNGCGTWEICGSPIHMIPPGESVRYLGVQVGPGRGVMEPDLIPTVHTWIERISEAPLKPSQRMRVLNSFALPRIIYQADLGKVTVTKLAQIDGIVRKAVKKWLHLSPSTCNGLLYSRNRDGGLGLLKLERLIPSVRTKRIYRMSRSPDIWTRRMTSHSVSKSDWEMLWVQAGGERGSAPVMGAVEAAPTDVERSPDYPDWRREENLAWSALRVQGVGADQFRGDRTSSSWIAEPASVGFAQRHWLAALALRAGVYPTREFLARGKEKSGAACRRCPARLESCSHILGQCPFVQANRIARHNKVCVLLATEAERFGWTVIREFRLEDAAGGLKIPDLVCKKADTVLIVDVTVRYEMDGETLKRAASEKVKHYLPVGQQITDKVGGRCFKVMGFPVGARGKWPASNNTVLAELGVPAGRMRTFARLVSRRTLLYSLDILRDFMREPAGRGTRVALIPAATGAANPKKKRKV

SEQ ID NO: 23 - protein, 1919 aa, Artificial Sequence
  source: /note=“Description of Artificial Sequence Synthetic polypeptide”
DKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSSGSETPGTSESATPESSGSGGSSGGSSGSETPGTSESATPESSGGSSGGSSILKILNEIASIGSTKQKQAILEKNKDNELLKRVYRLTYSRGLQYYIKKWPKPGIATQSFGMLTLTDMLDFIEFTLATRKLTGNAAIEELTGYITDGKKDDVEVLRRVMMRDLECGASVSIANKVWPGLIPEQPQMLASSYDEKGINKNIKFPAFAQLKADGARCFAEVRGDELDDVRLLSRAGNEYLGLDLLKEELIKMTAEARQIHPEGVLIDGELVYHEQVKKEPEGLDFLFDAYPENSKAKEFAEVAESRTASNGIANKSLKGTISEKEAQCMKFQVWDYVPLVEIYSLPAFRLKYDVRFSKLEQMTSGYDKVILIENQVVNNLDEAKVIYKKYIDQGLEGIILKNIDGLWENARSKNLYKFKEVIDVDLKIVGIYPHRKDPTKAGGFILESECGKIKVNAGSGLKDKAGVKSHELDRTRIMENQNYYIGKILECECNGWLKSDGRTDYVKLFLPIAIRLREDKTKANTFEDVFGDFHEVTGLPKKKRKV

SEQ ID NO: 24 - protein, 2267 aa, Artificial Sequence
  source: /note=“Description of Artificial Sequence Synthetic polypeptide”
PKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSSGSETPGTSESATPESSGGSSGGSSASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIYSGGSSGGSSGSETPGTSESATPESSGGSSGGSSTLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSPSGGSKRTADGSEFEPKKKRKV

SEQ ID NO: 25 - protein, 2252 aa, Artificial Sequence
  source: /note=“Description of Artificial Sequence Synthetic polypeptide”
PKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSSGSETPGTSESATPESSGGSSGGSSPSMKDARDWMKRMEDIGLEALGMNDFKLAYISDTYGSEIVYDRKFVRVANCDIEVTGDKFPDPMKAEYEIDAITHYDSIDDRFYVFDLLNSMYGSVSKWDAKLAAKLDCEGGDEVPQEILDRVIYMPFDNERDMLMEYINLWEQKRPAIFTGWNIEGFDVPYIMNRVKMILGERSMKRFSPIGRVKSKLIQNMYGSKEIYSIDGVSILDYLDLYKKFAFTNLPSFSLESVAQHETKKGKLPYDGPINKLRETNHQRYISYNIIDVESVQAIDKIRGFIDLVLSMSYYAKMPFSGVMSPIKTWDAIIFNSLKGEHKVIPQQGSHVKQSFPGAFVFEPKPIARRYIMSFDLTSLYPSIIRQVNISPETIRGQFKVHPIHEYIAGTAPKPSDEYSCSPNGWMYDKHQEGIIPKEIAKVFFQRKDWKKKMFAEEMNAEAIKKIIMKGAGSCSTKPEVERYVKFSDDFLNELSNYTESVLNSLIEECEKAATLANTNQLNRKILINSLYGALGNIHFRYYDLRNATAITIFGQVGIQWIARKINEYLNKVCGTNDEDFIAAGDTDSVYVCVDKVIEKVGLDRFKEQNDLVEFMNQFGKKKMEPMIDVAYRELCDYMNNREHLMHMDREAISCPPLGSKGVGGFWKAKKRYALNVYDMEDKRFAEPHLKIMGMETQQSSTPKAVQEALEESIRRILQEGEESVQEYYKNFEKEYRQLDYKVIAEVKTANDIAKYDDKGWPGFKCPFHIRGVLTYRRAVSGLGVAPILDGNKVMVLPLREGNPFGDKCIAWPSGTELPKEIRSDVLSWIDHSTLFQKSFVKPLAGMCESAGMDYEEKASLDFLFGPKKKRKV

SEQ ID NO: 26 - protein, 952 aa, Artificial Sequence
  source: /note=“Description of Artificial Sequence Synthetic polypeptide”
PKKKRKVMFKRKSTAELAAQMAKLNGNKGFSSEDKGEWKLKLDNAGNGQAVIRFLPSKNDEQAPFAILVNHGFKKNGKWYIETCSSTHGDYDSCPVCQYISKNDLYNTDNKEYSLVKRKTSYWANILVVKDPAAPENEGKVFKYRFGKKIWDKINAMIAVDVEMGETPVDVTCPWEGANFVLKVKQVSGFSNYDESKFLNQSAIPNIDDESFQKELFEQMVDLSEMTSKDKFKSFEELNTKFGQVMGTAVMGGAAATAAKKADKVADDLDAFNVDDFNTKTEDDFMSSSSGSSSSADDTDLDDLLNDLMADYSTVPPPSSGSAGGGGGGGGGGGVNDAFKDALQRARQIAAKIGGDAGTSLNSNDYGYGGQKRPLEDGDQPDAKKVAPQNDSFGTQLPPMHQQQSRSVMTEEYKVPDGMVGFIIGRGGEQISRIQQESGCKIQIAPDSGGLPERSCMLTGTPESVQSAKRLLDQIVEKGRPAPGFHHGDGPGNAVQEIMIPASKAGLVIGKGGETIKQLQERAGVKMVMIQDGPQNTGADKPLRITGDPYKVQQAKEMVLELIRDQGGFREVRNEYGSRIGGNEGIDVPIPRFAVGIVIGRNGEMIKKIQNDAGVRIQFKPDDGTTPERIAQITGPPDRCQHAAEIITDLLRSVQAGNPGGPGPGGRGRGRGQGNWNMGPPGGLQEFNFIVPTGKTGLIIGKGGETIKSISQQSGARIELQRNPPPNADPNMKLFTIRGTPQQIDYARQLIEEKIGGPVNPLGPPVPHGPHGVPGPHGPPGPPGPGTPMGPYNPAPYNPGPPGPAPHGPPAPYAPQGWGNAYPHWQQQAPPDPAKAGTDPNSAAWAAYYAHYYQQQAQPPPAAPAGAPTTTQTNGQGDQQNPAPAGQVDYTKAWEEYYKKMGQAVPAPTGAPPGGQPDYSAAWAEYYRQQAAYYAQTSPQGMPQHPPAPQGQ

SEQ ID NO: 27 - DNA, 368 bp, Artificial Sequence
  source: /note=“Description of Artificial Sequence Synthetic polynucleotide”
agagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgtggccccactgtggggtgggttttagagctagaaatagcaagttaaaataaggctagtccgttatcaacttgaaaaagtgggaccgagtcggtccaagatgccccacagtttttttt

SEQ ID NO: 28 - DNA, 379 bp, Artificial Sequence
  source: /note=“Description of Artificial Sequence Synthetic polynucleotide”
agagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgtggccccactgtggggtgggttttagagctagaaatagcaagttaaaataaggctagtccgttatcaacttgaaaaagtgggaccgagtcggtccaagatgccccacagtggggccactagttttttt

SEQ ID NO: 29 - protein, 20 aa, Artificial Sequence
  source: /note=“Description of Artificial Sequence Synthetic peptide”
AVKRPAATKKAGQAKKKKLD

SEQ ID NO: 30 - protein, 9 aa, Unknown
  source: /note=“Description of Unknown C-myc NLS sequence”
PAAKRVKLD

SEQ ID NO: 31 - protein, 25 aa, Artificial Sequence
  source: /note=“Description of Artificial Sequence Synthetic peptide”
MSRRRKANPTKLSENAKKLAKEVEN

SEQ ID NO: 32 - protein, 9 aa, Artificial Sequence
  source: /note=“Description of Artificial Sequence Synthetic peptide”
KLKIKRPVK

SEQ ID NO: 33 - protein, 5 aa, Artificial Sequence
  source: /note=“Description of Artificial Sequence Synthetic peptide”
KIPIK

SEQ ID NO: 34 - DNA, 23 bp, Artificial Sequence
  source: /note=“Description of Artificial Sequence Synthetic oligonucleotide”
gaaattaacaaactaatctgtgc

SEQ ID NO: 35 - RNA, 122 nt, Artificial Sequence
  source: /note=“Description of Artificial Sequence Synthetic polynucleotide”
guggccccacugugggguggguuuuagagcuagaaauagcaaguuaaaauaaggcuaguccguuaucaacuugaaaaagugggaccgagucgguccaagaugccccacaguggggccacuag

SEQ ID NO: 36 - DNA, 122 bp, Artificial Sequence
  source: /note=“Description of Artificial Sequence Synthetic oligonucleotide”
  source: /note=“Description of Combined DNA/RNA Molecule Synthetic oligonucleotide”
guggccccacugugggguggguuuuagagcuagaaauagcaaguuaaaauaaggcuaguccguuaucaacuugaaaaagugggaccgagucgguccaagatgccccacagtggggccactag

SEQ ID NO: 37 - DNA, 122 bp, Artificial Sequence; identical to SEQ ID NO: 36
  source: /note=“Description of Artificial Sequence Synthetic oligonucleotide”
  source: /note=“Description of Combined DNA/RNA Molecule Synthetic oligonucleotide”

SEQ ID NO: 38 - protein, 1228 aa, Artificial Sequence
  source: /note=“Description of Artificial Sequence Synthetic polypeptide”
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 39 - protein, 500 aa, Artificial Sequence
  source: /note=“Description of Artificial Sequence Synthetic polypeptide”
MEVQKTVMKTLSLRILRPLYSQEIEKEIKEEKERRKQAGGTGELDGGFYKKLEKKHSEMFSFDRLNLLLNQLQREIAKVYNHAISELYIATIAQGNKSNKHYISSIVYNRAYGYFYNAYIALGICSKVEANFRSNELLTQQSALPTAKSDNFPIVLHKQKGAEGEDGGFRISTEGSDLIFEIPIPFYEYNGENRKEPYKWVKKGGQKPVLKLILSTFRRQRNKGWAKDEGTDAEIRKVTEGKYQVSQIEINRGKKLGEHQKWFANFSIEQPIYERKPNRSIVGGLDVGIRSPLVCAINNSFSRYSVDSNDVFKFSKQVFAFRRRLLSKNSLKRKGHGAAHKLEPITEMTEKNDKFRKKIIERWAKEVTNFFVKNQVGIVQIEDLSTMKDREDHFFNQYLRGFWPYYQMQTLIENKLKEYGIEVKRVQAKYTSQLCSNPNCRYWNNYFNFEYRKVNKFPKFKCEKCNLEISADYNAARNLSTPDIEKFVAKATKGINLPEK

SEQ ID NO: 40 - DNA, 87 bp, Artificial Sequence
  source: /note=“Description of Artificial Sequence Synthetic oligonucleotide”
gaatttctactaagtgtagattcatctgtgcccctccctccctgaaattaacaaactaatctgtgcccctccaagcccaggtgaagg

SEQ ID NO: 41 - DNA, 87 bp, Artificial Sequence; identical to SEQ ID NO: 40
  source: /note=“Description of Artificial Sequence Synthetic oligonucleotide”

SEQ ID NO: 42 - DNA, 30 bp, Artificial Sequence
  source: /note=“Description of Artificial Sequence Synthetic oligonucleotide”
aaattaacaaactaatctgtgcccctccaa

SEQ ID NO: 43 - DNA, 30 bp, Artificial Sequence
  source: /note=“Description of Artificial Sequence Synthetic oligonucleotide”
gaaattaacaaactaatctgtgcccctcca

SEQ ID NO: 44 - DNA, 36 bp, Artificial Sequence
  source: /note=“Description of Artificial Sequence Synthetic oligonucleotide”
cctagtggccccactgtggggtggaggggacagata

SEQ ID NO: 45 - DNA, 36 bp, Artificial Sequence
  source: /note=“Description of Artificial Sequence Synthetic oligonucleotide”
tatctgtcccctccaccccacagtggggccactagg

SEQ ID NO: 46 - DNA, 121 bp, Artificial Sequence
  source: /note=“Description of Artificial Sequence Synthetic polynucleotide”
gtttcagttattcgtgaaaacgaatgaagtcactcttaaagtgagctgaaatcactaaaaattaagattgaacccggctactgactctgtcatccgggtttacttaaagatgcacagtggg

SEQ ID NO: 47 - DNA, 13 bp, Artificial Sequence
  source: /note=“Description of Artificial Sequence Synthetic oligonucleotide”
ttacttaaagatg

SEQ ID NO: 48 - DNA, 14 bp, Artificial Sequence
  source: /note=“Description of Artificial Sequence Synthetic oligonucleotide”
tttacttaaagatg